(19) United States
(12) Patent Application Publication
(10) Pub. No.: US 2026/0137806 A1
(43) Pub. Date: May 21, 2026

(54) INTERCALATING SUGAR MOLECULES

(71) Applicant: Xalud Therapeutics, Inc., Charlottesville, VA (US)

(72) Inventors: John Forsayeth, Oakland, CA (US); Raymond A. Chavez, Alameda, CA (US)

(73) Assignee: Xalud Therapeutics, Inc., Charlottesville, VA (US)

(21) Appl. No.: 18/866,886

(22) PCT Filed: May 19, 2023
(86) PCT No.: PCT/US2023/067218

§ 371 Date: Nov. 18, 2024

Related U.S. Application Data
(60) Provisional application No. 63/343,918, filed on May 19, 2022.

(51) Int. Cl. A61K 48/00; A61K 38/20; C07H 15/26

(52) U.S. Cl. CPC A61K 48/0033; A61K 38/2066; A61K 48/005; C07H 15/26

(57) ABSTRACT

Provided herein are compounds of Formula (I) (i.e., compounds of R¹-L-R²) or a pharmaceutically acceptable salt thereof, wherein R¹ is a substituted or unsubstituted sugar, substituted or unsubstituted amino sugar, or substituted or unsubstituted deoxy sugar; L is a linker; and R² is DNA-interacting agent (e.g., intercalating agent). Further provided are compositions, kits, methods, and uses comprising Formula (I). The compounds, compositions, kits, methods, and uses provided herein can be used for (i) delivering a compound or DNA (e.g., a plasmid) to a subject, and/or (ii) treating and/or prevention a disease or disorder.

### RELATED APPLICATIONS

The present application is a national stage filing under 35 U.S.C. § 371 of International PCT Application PCT/US2023/067218, filed May 19, 2023, which claims priority under 35 U.S.C. § 119(e) to U.S. Provisional Application, U.S. Ser. No. 63/343,918, filed May 19, 2022, each of which is incorporated herein by reference.

### REFERENCE TO AN ELECTRONIC SEQUENCE LISTING

The contents of the electronic sequence listing (X003270009US01-SUBSEQ-DCS.xml; Size: 47,049 bytes; and Date of Creation: Feb. 6, 2026) is herein incorporated by reference in its entirety.

### BACKGROUND OF THE INVENTION

Gene therapy aims to modify the expression of genes in a subject for therapeutic applications by delivering genetic material (e.g., DNA, RNA) to cells of the subject. There are various ways to introduce genetic material into the subject including via administration of therapeutic plasmids, viral vectors, or bacterial vectors. Although there are a variety of gene therapy products on the market, a challenge remains in improving the uptake of the genetic material into target cells.

### SUMMARY OF THE INVENTION

The present disclosure provides compounds of Formula (I) which include a sugar moiety (i.e., R¹) and a DNA-interacting moiety (i.e., R²). The compounds of Formula (I) bind to DNA (e.g., double-stranded DNA (e.g., plasmid DNA)) and, by virtue of the sugar, direct the DNA to cells that express specific sugar-binding receptors. Sugar-binding receptors include mannose receptors, mannose-6-phosphate (M6P) receptors found in various types of cells including, for example, microglia (Suh, H. S., et al. (2010) Am J Pathol 177(5): 2446) and macrophages (Rom, W. N. et al. (1991) Am J Respir Cell Mol Biol 4(6): 555; Shepherd, V. L. et al. (1984) J Biol Chem 259(4): 2257). Thus, the compounds of Formula (I) have an affinity for DNA and help facilitate uptake of DNA into target cells.

In one aspect, provided herein are compounds of Formula (I):

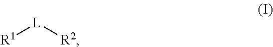

or a pharmaceutically acceptable salt thereof, wherein R¹ is a substituted or unsubstituted sugar, substituted or unsubstituted amino sugar, or substituted or unsubstituted deoxy sugar. L is a linker as defined herein; and R² is DNA-interacting agent. In some embodiments, the sugar is mannose or mannose 6-phosphate. In some embodiments, the DNA-interacting agent is a DNA intercalating agent. In certain embodiments, the DNA intercalating agent is quinacrine. In some embodiments, the compound of Formula (I) is of the formula:

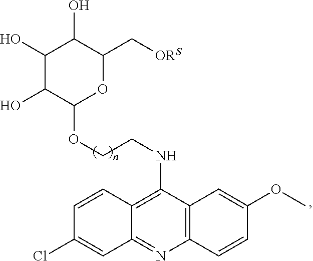

or a pharmaceutically acceptable salt thereof, wherein R^{S} and n are defined herein. In some embodiments, the compound of Formula (I) is of the formula:

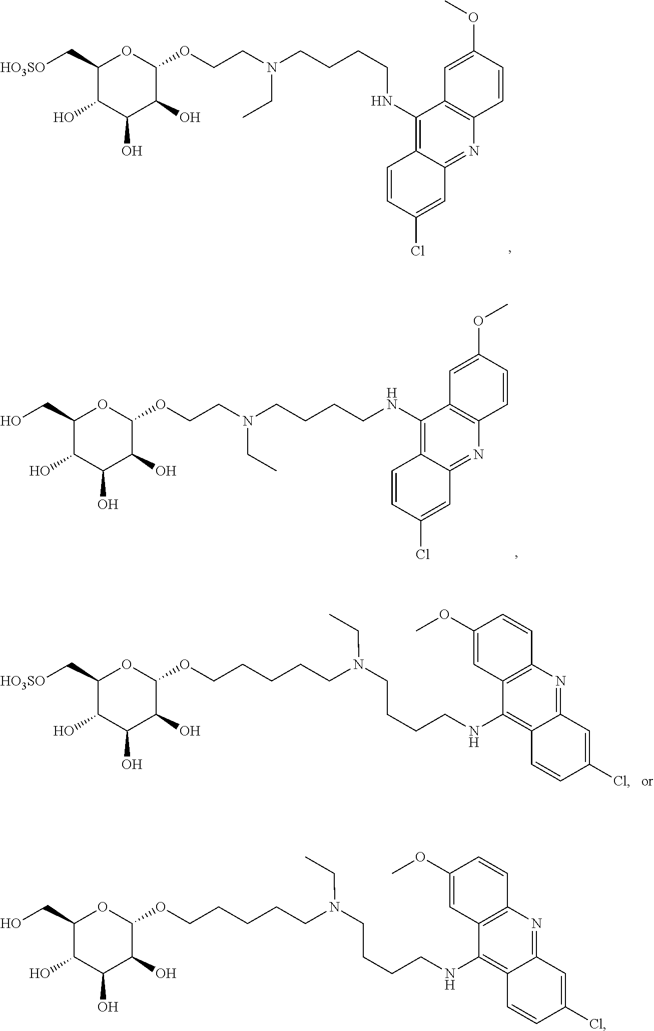

or a pharmaceutically acceptable salt thereof.

In another aspect, provided herein are compositions comprising a compound of Formula (I) and a pharmaceutically acceptable excipient.

In a further aspect, provided herein are compositions comprising a compound of Formula (I) and DNA. In some embodiments, the composition further comprises a pharmaceutically acceptable excipient.

In a further aspect, provided herein are compositions comprising a compound of Formula (I) and double-stranded DNA. In some embodiments, the composition further comprises a pharmaceutically acceptable excipient.

In another aspect, provided herein are compositions comprising a compound of Formula (I) and a plasmid. In some embodiments, the composition further comprises a pharmaceutically acceptable excipient. In some embodiments, the plasmid encodes a IL-10 polypeptide (e.g., wild type IL-10, mutant IL-10 (e.g., IL-10^{F129S})).

In a further aspect, provided herein are methods of delivering a compound of Formula (I) to subject, the method comprising administering an effective amount of a compound of Formula (I) or composition as described herein to a subject in need thereof.

In another aspect, provided herein are methods of delivering DNA to a subject, the method comprising administering an effective amount of a composition described herein to a subject in need thereof.

In another aspect, provided herein are methods of delivering double-stranded DNA to a subject, the method comprising administering an effective amount of a composition described herein to a subject in need thereof.

In a further aspect, provided herein are methods of delivering a plasmid to subject, the method comprising administering an effective amount of a composition described herein to a subject in need thereof.

In another aspect, provided herein are methods of treating or preventing a disease or disorder in a subject, the method comprising administering an effective amount of a composition described herein (e.g., a composition comprising a compound of Formula (I) and DNA (e.g., double-stranded DNA (e.g., plasmid DNA)) to a subject in need thereof. In some embodiments, the disease or disorder is an inflammatory disease, proliferative disease, autoimmune disease, hematological disease, genetic disease, neurological disease, painful condition, metabolic disorder, infectious disease, cardiovascular disease, cerebrovascular disease, tissue repair disorder, pulmonary disease, dermatological disease, bone disease, or hormonal disease. In some embodiments, the disease or disorder is an inflammatory disease. In some embodiments the inflammatory disease is an inflammatory disease of the joints (e.g., arthritis, facet syndrome, tendonitis, bursitis, inflammation of the ligament, synovitis, gout, or systemic lupus erythematosus). In some embodiments, the disease or disorder is an autoimmune disease. In certain embodiments, the autoimmune disease is inflammatory bowel disease (IBD), multiple sclerosis (MS), amyotrophic lateral sclerosis (ALS), rheumatoid arthritis (RA), systemic lupus erythematosus, or psoriasis.

In another aspect, provided herein are kits comprising a compound as described herein or composition described herein and instructions for administering to a subject the compound or composition.

The details of non-limiting embodiments of the disclosure are set forth in the Detailed Description of Certain Embodiments, as described below. Other features, objects, and advantages of the invention will be apparent from the Definitions. Figures, Examples, and Claims.

### BRIEF DESCRIPTION OF THE DRAWINGS

The following drawings provide non-limiting examples of the invention.

FIG. 1 provides a plasmid map of XT-150.

FIG. 2 is a wavelength versus absorbance graph used to study quinacrine-DNA binding. Quinacrine concentrations were held constant under low ionic conditions (10 mM Na⁺), and DNA plasmid was titrated. Absorbance was measured at 260 nm (DNA absorbance), 424 nm (quinacrine absorbance), 445 nm (quinacrine absorbance), and 461 nm (isobestic point). Changes in absorbance for quinacrine were monitored to demonstrate quinacrine-DNA complex formation.

FIG. 3 is a plasmid:quinacrine ratio versus absorbance graph used to study quinacrine-DNA binding saturation. The recovery of absorbance (marked with an arrow) indicates saturation. Saturation occurred at a plasmid/quinacrine ratio of 8 in 4 mM or 8 mM phosphate/citrate buffer.

FIG. 4 is a plasmid:quinacrine ratio versus absorbance graph used to study mannoquin compounds DNA binding saturation. For Mannoquin 36, saturation occurred at a plasmid/quinacrine ratio of 30 in 8 mM phosphate/citrate buffer.

FIG. 5 is a plasmid:quinacrine ratio versus absorbance graph used to study mannoquin compounds DNA binding saturation. For Mannoquin 37, saturation occurred at a plasmid/quinacrine ratio of 10-20 in 8 mM phosphate/citrate buffer.

FIG. 6 is a plasmid:quinacrine ratio versus absorbance graph used to study mannoquin compounds DNA binding saturation. For Mannoquin 38, saturation occurred at a plasmid/quinacrine ratio of 30-50 in 8 mM phosphate/citrate buffer.

FIG. 7 is a plasmid:quinacrine ratio versus absorbance graph used to study mannoquin compounds DNA binding saturation. For Mannoquin 36, saturation occurred at a plasmid/quinacrine ratio of 20-40 in 8 mM phosphate/citrate buffer.

FIG. 8 is a time versus absorbance graph used to study mannoquin-DNA complex stability. Mannoquin 36 appears to remain complexed to plasmid for 3 days.

FIG. 9 is a time versus absorbance graph used to study mannoquin-DNA complex stability. Mannoquin 37 appears to remain complexed to plasmid for 8 days at a saturation ratio of 10-20.

FIG. 10 is a time versus absorbance graph used to study mannoquin-DNA complex stability. Mannoquin 38 appears to remain complexed to plasmid for 8 days at a ratio of 60.

FIG. 11 is a time versus absorbance graph used to study mannoquin-DNA complex stability. Mannoquin 39 appears to remain complexed to plasmid for 8 days at a ratio of 30.

FIG. 12 shows plasmid DNA uptake in activated NR8383 rat alveolar macrophages after polymerase chain reaction (PCR) without and with lipopolysaccharides (LPS) treatment.

### DEFINITIONS

For convenience, certain terms employed herein, in the specification, examples and appended claims are collected herein.

Unless otherwise required by context, singular terms shall include pluralities, and plural terms shall include the singular.

The language “in some embodiments” and the language “in certain embodiments” are used interchangeably.

The following definitions are more general terms used throughout the present application:

The singular terms “a,” “an,” and “the” include plural references unless the context clearly indicates otherwise. Similarly, the word “or” is intended to include “and” unless the context clearly indicates otherwise.

Other than in the examples, or where otherwise indicated, all numbers expressing quantities of ingredients or reaction conditions used herein should be understood as modified in all instances by the term “about.” “About” and “approximately” shall generally mean an acceptable degree of error for the quantity measured given the nature or precision of the measurements. Exemplary degrees of error are within 20 percent (%), typically, within 10%, or more typically, within 5%, 4%, 3%, 2% or 1% of a given value or range of values.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience. New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel. Ed., Univ. of Notre Dame Press, Notre Dame, IN 1972). The invention additionally encompasses compounds as individual isomers substantially free of other isomers, and alternatively, as mixtures of various isomers.

The term “about X” or “approximately X,” where X is a number or percentage, refers to a number or percentage that is between 99.5% and 100.5%, between 99% and 101%, between 98% and 102%, between 97% and 103%, between 96% and 104%, between 95% and 105%, between 92% and 108%, or between 90% and 110%, inclusive, of X.

In a formula, the bond [symbol] is a single bond, the dashed line [symbol] is a single bond or absent, and the bond [symbol] or [symbol] is a single or double bond.

When a range of values (“range”) is listed, it encompasses each value and sub-range within the range. A range is inclusive of the values at the two ends of the range unless otherwise provided. For example, “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the haloalkyl moiety has 1 to 8 carbon atoms (“C₁₋₄ haloalkyl”). In some embodiments, the haloalkyl moiety has 1 to 7 carbon atoms (“C₁₋₇ haloalkyl”).In some embodiments, the haloalkyl moiety has 1 to 6 carbon atoms (“C₁₋₆ haloalkyl”). In some embodiments, the haloalkyl moiety has 1 to 5 carbon atoms (“C₁₋₅ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃. —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and lor 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in 1-butenyl). Examples of C₁₋₄ alkenyl groups include methylidenyl (C₁), ethenyl (C₂) 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

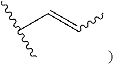

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”). In certain embodiments, a heteroalkenyl group refers to a group having from 1 to 12 carbon atoms, at least one double bond, and 1 or more heteroatoms within the parent chain (“heteroC₁₋₁₂ alkenyl”). In certain embodiments, a heteroalkenyl group refers to a group having from 1 to 11 carbon atoms, at least one double bond, and 1 or more heteroatoms within the parent chain (“heteroC₁₋₁₁ alkenyl”). In certain embodiments, a heteroalkenyl group refers to a group having from 1 to 10 carbon atoms, at least one double bond, and 1 or more heteroatoms within the parent chain (“heteroC₁₋₁₀ alkenyl”). In some embodiments, a heteroalkenyl group has 1 to 9 carbon atoms at least one double bond, and 1 or more heteroatoms within the parent chain (“heteroC₁₋₉ alkenyl”). In some embodiments, a heteroalkenyl group has 1 to 8 carbon atoms, at least one double bond, and 1 or more heteroatoms within the parent chain (“heteroC₁₋₈ alkenyl”). In some embodiments, a heteroalkenyl group has 1 to 7 carbon atoms, at least one double bond, and 1 or more heteroatoms within the parent chain (“heteroC₁₋₇ alkenyl”). In some embodiments, a heteroalkenyl group has 1 to 6 carbon atoms, at least one double bond, and 1 or more heteroatoms within the parent chain (“heteroC₁₋₆ alkenyl”). In some embodiments, a heteroalkenyl group has 1 to 5 carbon atoms, at least one double bond, and 1 or 2 heteroatoms within the parent chain (“heteroC₁₋₅ alkenyl”). In some embodiments, a heteroalkenyl group has 1 to 4 carbon atoms, at least one double bond, and 1 or 2 heteroatoms within the parent chain (“heteroC₁₋₄ alkenyl”). In some embodiments, a heteroalkenyl group has 1 to 3 carbon atoms, at least one double bond, and 1 heteroatom within the parent chain (“heteroC₁₋₃ alkenyl”). In some embodiments, a heteroalkenyl group has 1 to 2 carbon atoms, at least one double bond, and 1 heteroatom within the parent chain (“heteroC₁₋₂ alkenyl”). In some embodiments, a heteroalkenyl group has 1 to 6 carbon atoms, at least one double bond, and 1 or 2 heteroatoms within the parent chain (“heteroC₁₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₁₋₂₀ alkenyl. In certain embodiments, the heteroalkenyl group is a substituted heteroC₁₋₂₀ alkenyl.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). The one or more carbon-carbon triple bonds can be internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”). In certain embodiments, a heteroalkynyl group refers to a group having from 1 to 10 carbon atoms, at least one triple bond, and 1 or more heteroatoms within the parent chain (“heteroC₁₋₁₀ alkynyl”). In some embodiments, a heteroalkynyl group has 1 to 9 carbon atoms, at least one triple bond, and 1 or more heteroatoms within the parent chain (“heteroC₁₋₉ alkynyl”). In some embodiments, a heteroalkynyl group has 1 to 8 carbon atoms, at least one triple bond, and 1 or more heteroatoms within the parent chain (“heteroC₁₋₈ alkynyl”). In some embodiments, a heteroalkynyl group has 1 to 7 carbon atoms, at least one triple bond, and 1 or more heteroatoms within the parent chain (“heteroC₁₋₇ alkynyl”). In some embodiments, a heteroalkynyl group has 1 to 6 carbon atoms, at least one triple bond, and 1 or more heteroatoms within the parent chain (“heteroC₁₋₆ alkynyl”). In some embodiments, a heteroalkynyl group has 1 to 5 carbon atoms, at least one triple bond, and 1 or 2 heteroatoms within the parent chain (“heteroC₁₋₅ alkynyl”). In some embodiments, a heteroalkynyl group has 1 to 4 carbon atoms, at least one triple bond, and lor 2 heteroatoms within the parent chain (“heteroC₁₋₄ alkynyl”). In some embodiments, a heteroalkynyl group has 1 to 3 carbon atoms, at least one triple bond, and 1 heteroatom within the parent chain (“heteroC₁₋₃ alkynyl”). In some embodiments, a heteroalkynyl group has 1 to 2 carbon atoms, at least one triple bond, and 1 heteroatom within the parent chain (“heteroC₁₋₂ alkynyl”). In some embodiments, a heteroalkynyl group has 1 to 6 carbon atoms, at least one triple bond, and 1 or 2 heteroatoms within the parent chain (“heteroC₁₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₁₋₂₀ alkynyl. In certain embodiments, the heteroalkynyl group is a substituted heteroC₁₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₇) and cyclohexyl (C₈). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetra-hydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₄₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 n electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not limited in any manner by the exemplary substituents described herein.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{aa})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc}; wherein:
each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{b} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₁₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{aa}, —SO₂N(Rf)₂, —SO₂R^{aa}, —SO₂OR^{aa}, —OSO₂R^{aa}, —S(═O)R^{aa}, —Si(R^{aa})₃, —OSi(R^{aa})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S; wherein X⁻ is a counterion;
each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R^{gg} substituents can be joined to form ═O or ═S; and
each X⁻ is a counterion.

In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES. TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz. Fmoc, trifluoroacetyl, triphenylmethyl, acetyl, or Ts).

In certain embodiments, the molecular weight of a carbon atom substituent is lower than 250, lower than 200, lower than 150, lower than 100, or lower than 50 g/mol. In certain embodiments, a carbon atom substituent consists of carbon, hydrogen, fluorine, chlorine, bromine, iodine, oxygen, sulfur, nitrogen, and/or silicon atoms. In certain embodiments, a carbon atom substituent consists of carbon, hydrogen, fluorine, chlorine, bromine, iodine, oxygen, sulfur, and/or nitrogen atoms. In certain embodiments, a carbon atom substituent consists of carbon, hydrogen, fluorine, chlorine, bromine, and/or iodine atoms. In certain embodiments, a carbon atom substituent consists of carbon, hydrogen, fluorine, and/or chlorine atoms.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{b})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{aa})₂, —OP(R^{aa})₃⁺X⁻, —OP(OR^{cc})₂, —OP(OR^{cc})₃X⁻, —OP(═O)(R^{aa})₂, —OP(═O)(OR^{cc})₂, and —OP(═O)(N(R^{aa}))₂, wherein X⁻, R^{aa}, R^{bb}, and R^{cc} are as defined herein.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═S)OR^{aa}, —SC(═S) N(R^{b})₂, —SC(═O)SR^{aa}, —SC(═O)OR^{aa}, —SC(═O)N(R^{bb})₂, and —SC(═O)R^{aa}, wherein R^{aa} and R^{cc} are as defined herein.

The term “amino” refers to the group —NH₂. The term “substituted amino,” by extension, refers to a monosubstituted amino, a disubstituted amino, or a trisubstituted amino.

In certain embodiments, the “substituted amino” is a monosubstituted amino or a disubstituted amino group.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “silyl” refers to the group —Si(R^{aa})₃, wherein R^{aa} is as defined herein.

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{aa})₂, —CN, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{aa})₂, —SO₂N(R^{cc})₂, —SO₂R^{aa}, —SO₂OR^{aa}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀alkenyl, C₁₋₂₀alkynyl, hetero C₁₋₂₀alkyl, hetero C₁₋₂₀alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that include the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbarnate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbanate (t-Bumeoc), 242′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbanate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentanethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives. N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine. N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₋₆ alkyl. —C(═O)R^{aa}, —CO₂R^{aa}, —C(═O)N(R^{bb})₂, or an oxygen protecting group, wherein R^{bb} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8.8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3′″-[N-(imidazolylmethyl)]trityl Ether (IDTr-OR), 4,4′-Dimethoxy-3′″-[N-(imidazolylethyl)carbamoyl]trityl Ether (IETr-OR), 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate. S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES. TMS. MOM, THP, t-Bu, Bn, allyl, acetyl, pivaloyl, or benzoyl.

In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group, wherein R^{bb} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each sulfur atom substituent is independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). In some embodiments, each sulfur protecting group is selected from the group consisting of —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methanesulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]4]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻HPO₄²⁻, PO₄³⁻, B₄O₇²⁻, SO₄²⁻—, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

These and other exemplary substituents are described in more detail in the Detailed Description, Examples, and Claims. The invention is not limited in any manner by the above exemplary listing of substituents.

As used herein, the term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of this invention include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N′(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “crystalline” or “crystalline form” refers to a solid form substantially exhibiting three-dimensional order. In certain embodiments, a crystalline form of a solid is a solid form that is substantially not amorphous. In certain embodiments, the X-ray powder diffraction (XRPD) pattern of a crystalline form includes one or more sharply defined peaks.

The term “amorphous” or “amorphous form” refers to a form of a solid (“solid form”), the form substantially lacking three-dimensional order. In certain embodiments, an amorphous form of a solid is a solid form that is substantially not crystalline. In certain embodiments, the X-ray powder diffraction (XRPD) pattern of an amorphous form includes a wide scattering band with a peak at 20 of, e.g., between 2° and 70°, inclusive, using CuKα radiation. In certain embodiments, the XRPD pattern of an amorphous form further includes one or more peaks attributed to crystalline structures. In certain embodiments, the maximum intensity of any one of the one or more peaks attributed to crystalline structures observed at a 2θ of between 2° and 70°, inclusive, is not more than 300-fold, not more than 100-fold, not more than 30-fold, not more than 10-fold, or not more than 3-fold of the maximum intensity of the wide scattering band. In certain embodiments, the XRPD pattern of an amorphous form includes no peaks attributed to crystalline structures.

The term “co-crystal” refers to a crystalline structure comprising at least two different components (e.g., a compound disclosed herein and an acid), wherein each of the components is independently an atom, ion, or molecule. In certain embodiments, none of the components is a solvent. In certain embodiments, at least one of the components is a solvent. A co-crystal of a compound disclosed herein and an acid is different from a salt formed from a compound disclosed herein and the acid. In the salt, a compound disclosed herein is complexed with the acid in a way that proton transfer (e.g., a complete proton transfer) from the acid to a compound disclosed herein easily occurs at room temperature. In the co-crystal, however, a compound disclosed herein is complexed with the acid in a way that proton transfer from the acid to a compound disclosed herein does not easily occur at room temperature. In certain embodiments, in the co-crystal, there is no proton transfer from the acid to a compound disclosed herein. In certain embodiments, in the co-crystal, there is partial proton transfer from the acid to a compound disclosed herein. Co-crystals may be useful to improve the properties (e.g., solubility, stability, and ease of formulation) of a compound disclosed herein.

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The terms “glycan,” “sugar,” “carbohydrate,” or “saccharide,” are used interchangeably herein and refers to an aldehydic or ketonic derivative of polyhydric alcohols. Carbohydrates include compounds with relatively small molecules (e.g., sugars) as well as macromolecular or polymeric substances (e.g., starch, glycogen, and cellulose polysaccharides). The term “sugar” refers to monosaccharides, disaccharides, or polysaccharides. Monosaccharides are the simplest carbohydrates in that they cannot be hydrolyzed to smaller carbohydrates. Most monosaccharides can be represented by the general formula C_{y}H_{2y}O_{y} (e.g., C₆H₁₂O₆ (a hexose such as glucose)), wherein y is an integer equal to or greater than 3. Certain polyhydric alcohols not represented by the general formula described above may also be considered monosaccharides. For example, deoxyribose is of the formula C₅H₁₀O₄ and is a monosaccharide. Monosaccharides usually consist of five or six carbon atoms and are referred to as pentoses and hexoses, receptively. If the monosaccharide contains an aldehyde it is referred to as an aldose; and if it contains a ketone, it is referred to as a ketose. Monosaccharides may also consist of three, four, or seven carbon atoms in an aldose or ketose form and are referred to as trioses, tetroses, and heptoses, respectively. Glyceraldehyde and dihydroxyacetone are considered to be aldotriose and ketotriose sugars, respectively. Examples of aldotetrose sugars include erythrose and threose; and ketotetrose sugars include erythrulose. Aldopentose sugars include ribose, arabinose, xylose, and lyxose; and ketopentose sugars include ribulose, arabulose, xylulose, and lyxulose. Examples of aldohexose sugars include glucose (for example, dextrose), mannose, galactose, allose, altrose, talose, gulose, idose, desosamine, and mycaminose; and ketohexose sugars include fructose, psicose, sorbose, and tagatose. Ketoheptose sugars include sedoheptulose. Each carbon atom of a monosaccharide bearing a hydroxyl group (—OH), with the exception of the first and last carbons, is asymmetric, making the carbon atom a stereocenter with two possible configurations (R or S). Because of this asymmetry, a number of isomers may exist for any given monosaccharide formula. The aldohexose d-glucose, for example, has the formula C₆H₁₂O₆, of which all but two of its six carbons atoms are stereogenic, making d-glucose one of the 16 (i.e., 2V) possible stereoisomers. The assignment of d or 1 is made according to the orientation of the asymmetric carbon furthest from the carbonyl group: in a standard Fischer projection if the hydroxyl group is on the right the molecule is a d sugar, otherwise it is an 1 sugar. The aldehyde or ketone group of a straight-chain monosaccharide will react reversibly with a hydroxyl group on a different carbon atom to form a hemiacetal or hemiketal, forming a heterocyclic ring with an oxygen bridge between two carbon atoms. Rings with five and six atoms are called furanose and pyranose forms, respectively, and exist in equilibrium with the straight-chain form. During the conversion from the straight-chain form to the cyclic form, the carbon atom containing the carbonyl oxygen, called the anomeric carbon, becomes a stereogenic center with two possible configurations: the oxygen atom may take a position either above or below the plane of the ring. The resulting possible pair of stereoisomers is called anomers. In an a anomer, the —OH substituent on the anomeric carbon rests on the opposite side (trans) of the ring from the —CH₂OH side branch. The alternative form, in which the —CH₂OH substituent and the anomeric hydroxyl are on the same side (cis) of the plane of the ring, is called a β anomer. A carbohydrate including two or more joined monosaccharide units is called a disaccharide or polysaccharide (e.g., a trisaccharide), respectively. The two or more monosaccharide units bound together by a covalent bond known as a glycosidic linkage formed via a dehydration reaction, resulting in the loss of a hydrogen atom from one monosaccharide and a hydroxyl group from another. Exemplary disaccharides include sucrose, lactulose, lactose, maltose, isomaltose, trehalose, cellobiose, xylobiose, laminaribiose, gentiobiose, mannobiose, melibiose, nigerose, or rutinose. Exemplary trisaccharides include, but are not limited to, isomaltotriose, nigerotriose, maltotriose, melezitose, maltotriulose, raffinose, and kestose. The term carbohydrate also includes other natural or synthetic stereoisomers of the carbohydrates described herein. In some embodiments, the sugar is erythrose, threose, erythulose, arabinose, lyxose, ribose, xylose, ribulose, xylulose, allose, altrose, galactose, glucose, gulose, idose, mannose, talose, fructose, psicose, sorbose, tagatose, fucose, fuculose, rhamnose, mannoheptulose, sedoheptulose, or derivatives thereof. In some embodiments, the sugar is erythrose, threose, erythulose, arabinose, lyxose, ribose, xylose, ribulose, xylulose, allose, altrose, galactose, glucose, gulose, idose, mannose, talose, fructose, psicose, sorbose, tagatose, fucose, fuculose, rhamnose, mannoheptulose, sedoheptulose, and/or derivatives thereof (e.g., N-acetylglucosamine. N-acetylgalactosamine, glucuronic acid, chondroitin sulfate, hyaluronic acid, etc.). In some embodiments, the sugar is erythrose, threose, erythulose, arabinose, lyxose, ribose, xylose, ribulose, xylulose, allose, altrose, galactose, glucose, gulose, idose, mannose, talose, fructose, psicose, sorbose, tagatose, fucose, fuculose, rhamnose, mannoheptulose, or sedoheptulose. Substituted sugars include, but are not limited to, sugars where the hydrogen of a hydroxyl group on a sugar has been replaced with a non-hydrogen group. For example, substituted sugars include phosphorylated sugars (i.e., conversion of —OH on a sugar to —OPO₃H₂) and sulphated sugars (i.e., conversion of —OH on a sugar to —OSO₃H).

The term “amino sugar” refers to sugar derivatives wherein a hydroxyl group of the sugar is replaced with an amine or amide group. In some embodiments, the amino sugar is N-acetylglucosamine, N-acetylgalactosamine, glucuronic acid, derivatives thereof, and/or combinations thereof (e.g. chondroitin sulfate, hyaluronic acid, the repeat unit of chondroitin sulfate, the repeat unit of hyaluronic acid, etc.). Substituted amino sugars include, but are not limited to, amino sugars where the hydrogen of a hydroxyl group, amino group, or amide group on an amino sugar has been replaced with a non-hydrogen group. For example, substituted amino sugars include phosphorylated amino sugars (i.e., conversion of —OH on a sugar to —OPO₃H₂) and sulphated amino sugars (i.e., conversion of —OH on a sugar to —OSO₃H).

The term “deoxy sugar” refers to sugar derivatives wherein a hydroxyl group of the sugar is replaced with a hydrogen atom. In some embodiments, the deoxy sugar refers to fucose, fuculose, or rhamnose. Substituted deoxy sugars include, but are not limited to, deoxy sugars where the hydrogen of a hydroxyl group on a deoxy sugar has been replaced with a non-hydrogen group. For example, substituted deoxy sugars include phosphorylated deoxy sugars (i.e., conversion of —OH on a sugar to —OPO₃H₂) and sulphated deoxy sugars (i.e., conversion of —OH on a sugar to —OSO₃H).

The term “DNA-interacting agent” refers to a molecule that can interact with DNA. DNA-interacting agents may interact with DNA via covalent or non-covalent bonds. Examples of non-covalent bonds include electrostatic (e.g., ionic bonds), π-effects (e.g., π-stacking, polar-π interactions, π donor-acceptor interactions, anion/cation-π interactions, and CH—π interactions), van der Waals forces, dipole-dipole bonds (e.g., hydrogen bonding), and hydrophobic effects.

The term “DNA intercalating agent” refers to a molecule that can insert between adjacent base pairs of double stranded DNA. Intercalating agents typically but not necessarily, contain planar polyaromatic rings and/or cationic substituents. Intercalation between adjacent base pairs may be full or partial. For example, quinacrine is a DNA intercalating agent.

The terms “composition” and “formulation” are used interchangeably.

The term “excipient” or “diluent” refers to an inert substance added to a therapeutic composition to facilitate administration of a compound of Formula (I), DNA, and/or therapeutic IL-10^{F129S} expression constructs provided using the dosing regimen described herein. Examples, without limitation, of excipients include saline, calcium carbonate, calcium phosphate, various sugars and types of starch, cellulose derivatives, gelatin, hyaluronic acid (optionally formulated with a surfactant), Plumoic F-68, vegetable oils, and polyethylene glycols. Further examples of diluents and excipients are provided herein. In some embodiments, the diluent is phosphate-buffered saline (pH 7.4).

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “target tissue” refers to any biological tissue of a subject (including a group of cells, a body part, or an organ) or a part thereof, including blood and/or lymph vessels, which is the object to which a compound, particle, and/or composition of the invention is delivered. A target tissue may be an abnormal or unhealthy tissue, which may need to be treated. A target tissue may also be a normal or healthy tissue that is under a higher than normal risk of becoming abnormal or unhealthy, which may need to be prevented. In certain embodiments, the target tissue is the liver. In certain embodiments, the target tissue is the bowel. In certain embodiments, the target tissue is the lung. In certain embodiments, the target tissue is the eye. In certain embodiments, the target tissue is the gum. In certain embodiments, the target tissue is the skin. In certain embodiments, the target tissue is one or more joints. A “non-target tissue” is any biological tissue of a subject (including a group of cells, a body part, or an organ) or a part thereof, including blood and/or lymph vessels, which is not a target tissue.

The term “target cell” refers to a cell that binds R¹. In some embodiments, the target cell expresses a receptor that binds a sugar. In some embodiments, the target cell expresses a glycan-binding receptor. In some embodiments, the target cell expresses a sugar-binding receptor. In some embodiments, the cell is a cell that expresses syndecan, α-dystoglycan CD44, CD44, glypicans, NG2, RPTP-σ, GPI-brevican, RHAMM, Toll-like, IGF-II, mannose (i.e., CD206), or mannose-6-phosphate receptors. In some embodiments, the target cell expresses a IGF-II receptor (insulin-like growth factor 2 receptor). In certain embodiments, the target cell expresses a mannose receptor. In certain embodiments, the target cell expresses a mannose-6-phosphate receptor.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The terms “condition,” “disease,” and “disorder” are used interchangeably.

An “effective amount” of a composition (i.e., DNA (e.g., double-stranded DNA (e.g., plasmid DNA)) therein) described herein refers to an amount sufficient to elicit the desired biological response. An effective amount of a composition (i.e., DNA (e.g., double-stranded DNA (e.g., plasmid DNA)) therein) described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of DNA (e.g., double-stranded DNA (e.g., plasmid DNA)) described herein in a single dose. In certain embodiments, an effective amount is the combined amounts of DNA (e.g., double-stranded DNA (e.g., plasmid DNA)) described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of DNA (e.g., double-stranded DNA (e.g., plasmid DNA)) for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of DNA (e.g., double-stranded DNA (e.g., plasmid DNA)) per unit dosage form.

In certain embodiments, the DNA (e.g., double-stranded DNA (e.g., plasmid DNA)) may be administered orally or parenterally at dosage levels sufficient to deliver from about 0.0001 mg/kg to about 100 mg/kg, from about 0.0005 mg/kg to about 50 mg/kg, from about 0.001 mg/kg to about 50 mg/kg, from about 0.01 mg/kg to about 50 mg/kg, from about 0.1 mg/kg to about 40 mg/kg, from about 0.5 mg/kg to about 30 mg/kg, from about 0.0001 mg/kg to about 10 mg/kg, preferably from about 0.001 mg/kg to about 10 mg/kg, preferably from about 0.01 mg/kg to about 10 mg/kg, from about 0.1 mg/kg to about 10 mg/kg, from about 0.0001 mg/kg to about 1 mg/kg, preferably from about 0.001 mg/kg to about 1 mg/kg, preferably from about 0.01 mg/kg to about 1 mg/kg, from about 0.1 mg/kg to about 1 mg/kg, from about 0.0001 mg/kg to about 0.1 mg/kg, preferably from about 0.001 mg/kg to about 0.1 mg/kg, preferably from about 0.01 mg/kg to about 0.1 mg/kg, from about 0.1 mg/kg to about 0.1 mg/kg, and from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions (i.e., DNA (e.g., double-stranded DNA (e.g., plasmid DNA)) therein) to an adult. The amount to be administered to, for example, a child or an adolescent can be determined by a medical practitioner or person skilled in the art and can be lower or the same as that administered to an adult.

A “therapeutically effective amount” of a composition (i.e., DNA (e.g., double-stranded DNA (e.g., plasmid DNA)) therein) described herein is an amount sufficient to provide a therapeutic benefit in the treatment of a condition or to delay or minimize one or more symptoms associated with the condition. A therapeutically effective amount of a composition (i.e., DNA (e.g., double-stranded DNA (e.g., plasmid DNA)) therein) means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for increasing the uptake of DNA (e.g., a double-stranded DNA (e.g., plasmid DNA (e.g., XT-150))) into a cell. In certain embodiments, a therapeutically effective amount is an amount sufficient for treating or preventing a disease or disorder as provided herein. In certain embodiments, a therapeutically effective amount is an amount sufficient for increasing the uptake of DNA (e.g., a plasmid (e.g., XT-150)) into a cell and treating a disease or disorder.

A “prophylactically effective amount” of a composition (i.e., DNA (e.g., double-stranded DNA (e.g., plasmid DNA)) therein) described herein is an amount sufficient to prevent a condition, or one or more symptoms associated with the condition or prevent its recurrence. A prophylactically effective amount of a composition (i.e., DNA (e.g., double-stranded DNA (e.g., plasmid DNA)) therein) means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for increasing the uptake of DNA (e.g., a plasmid (e.g., XT-150)) into a cell. In certain embodiments, a prophylactically effective amount is an amount sufficient for treating or preventing a disease or disorder as provided herein. In certain embodiments, a prophylactically effective amount is an amount sufficient for increasing the uptake of DNA (e.g., a plasmid (e.g., XT-150)) into a cell and treating or preventing a disease or disorder as provided herein.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

As used herein, the term “increasing the uptake” in the context of the therapeutic compositions provided herein, for example, refers to a increasing the amount of genetic material (e.g., the genetic material in the composition (e.g., DNA (e.g., double-stranded DNA (e.g., plasmid DNA)))) in a cell in the subject or biological sample.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells: 2) the pathological migration of cells from their normal location (e.g., metastasis of neoplastic cells): 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis.” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer, anal cancer: angiosarcoma (e.g., lymphangiosarcoma, lymphangioendotheliosarcoma, hemangiosarcoma); appendix cancer; benign monoclonal gammopathy; biliary cancer (e.g., cholangiocarcinoma): bladder cancer, breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer, carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer, epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer, gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL. T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma. Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma): lung cancer (e.g., bronchogenic carcinoma, small cell lung cancer (SCLC), non-small cell lung cancer (NSCLC), adenocarcinoma of the lung); leiomyosarcoma (LMS): mastocytosis (e.g., systemic mastocytosis); muscle cancer, myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The terms “inflammatory disease” and “inflammatory condition” are used interchangeably herein, and refer to a disease or condition caused by, resulting from, or resulting in inflammation. Inflammatory diseases and conditions include those diseases, disorders or conditions that are characterized by signs of pain (dolor, from the generation of noxious substances and the stimulation of nerves), heat (calor, from vasodilatation), redness (rubor, from vasodilatation and increased blood flow), swelling (tumor, from excessive inflow or restricted outflow of fluid), and/or loss of function (functio laesa, which can be partial or complete, temporary or permanent). Inflammation takes on many forms and includes, but is not limited to, acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation.

Additional exemplary inflammatory conditions include, but are not limited to, inflammation associated with acne, anemia (e.g., aplastic anemia, hemolytic autoimmune anemia), asthma, arteritis (e.g., polyarteritis, temporal arteritis, periarteritis nodosa, Takayasu's arteritis), arthritis (e.g., crystalline arthritis, osteoarthritis, psoriatic arthritis, gouty arthritis, reactive arthritis, rheumatoid arthritis and Reiter's arthritis), ankylosing spondylitis, amylosis, amyotrophic lateral sclerosis, autoimmune diseases, allergies or allergic reactions, atherosclerosis, bronchitis, bursitis, chronic prostatitis, conjunctivitis, Chagas disease, chronic obstructive pulmonary disease, cermatomyositis, diverticulitis, diabetes (e.g., type I diabetes mellitus, Type II diabetes mellitus), a skin condition (e.g., psoriasis, eczema, burns, dermatitis, pruritus (itch)), endometriosis, Guillain-Barre syndrome, infection, ischemic heart disease, Kawasaki disease, glomerulonephritis, gingivitis, hypersensitivity, headaches (e.g., migraine headaches, tension headaches), ileus (e.g., postoperative ileus and ileus during sepsis), idiopathic thrombocytopenic purpura, interstitial cystitis (painful bladder syndrome), gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischemic colitis, diversion colitis, Behcet's syndrome, indeterminate colitis) and inflammatory bowel syndrome (IBS)), lupus, multiple sclerosis, morphea, myasthenia gravis, myocardial ischemia, nephrotic syndrome, pemphigus vulgaris, pernicious anemia, peptic ulcers, polymyositis, primary biliary cirrhosis, neuroinflammation associated with brain disorders (e.g., Parkinson's disease, Huntington's disease, and Alzheimer's disease), prostatitis, chronic inflammation associated with cranial radiation injury, pelvic inflammatory disease, reperfusion injury, regional enteritis, rheumatic fever, systemic lupus erythematosus, scleroderma, sarcoidosis, spondyloarthopathies, Sjogren's syndrome, thyroiditis, transplantation rejection, tendonitis, trauma or injury (e.g., frostbite, chemical irritants, toxins, scarring, burns, physical injury), vasculitis, vitiligo and Wegener's granulomatosis. In certain embodiments, the inflammatory disorder is selected from arthritis (e.g., rheumatoid arthritis), inflammatory bowel disease, inflammatory bowel syndrome, asthma, psoriasis, endometriosis, interstitial cystitis and prostatitis. In certain embodiments, the inflammatory condition is an acute inflammatory condition (e.g., for example, inflammation resulting from infection). In certain embodiments, the inflammatory condition is a chronic inflammatory condition (e.g., conditions resulting from asthma, arthritis and inflammatory bowel disease). The compounds may also be useful in treating inflammation associated with trauma and non-inflammatory myalgia. The compounds disclosed herein may also be useful in treating inflammation associated with cancer

An “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response. Exemplary autoimmune diseases include, but are not limited to, glomerulonephritis, Goodpasture's syndrome, necrotizing vasculitis, lymphadenitis, peri-arteritis nodosa, systemic lupus erythematosis, rheumatoid arthritis, psoriatic arthritis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme disease, Guillain-Barr syndrome, Hashimoto's thyroiditis, and cardiomyopathy.

A “hematological disease” includes a disease which affects a hematopoietic cell or tissue. Hematological diseases include diseases associated with aberrant hematological content and/or function. Examples of hematological diseases include diseases resulting from bone marrow irradiation or chemotherapy treatments for cancer, diseases such as pernicious anemia, hemorrhagic anemia, hemolytic anemia, aplastic anemia, sickle cell anemia, sideroblastic anemia, anemia associated with chronic infections such as malaria, trypanosomiasis, hantavirus, hepatitis virus or other viruses, myelophthisic anemias caused by marrow deficiencies, renal failure resulting from anemia, anemia, polycythemia, infectious mononucleosis (EVI), acute non-lymphocytic leukemia (ANLL), acute myeloid leukemia (AML), acute promyelocytic leukemia (APL), acute myelomonocytic leukemia (AMMoL), polycythemia vera, lymphoma, acute lymphocytic leukemia (ALL), chronic lymphocytic leukemia, Wilms tumor, Ewing's sarcoma, retinoblastoma, hemophilia, disorders associated with an increased risk of thrombosis, herpes, thalassemia, antibody-mediated disorders such as transfusion reactions and erythroblastosis, mechanical trauma to red blood cells such as micro-angiopathic hemolytic anemias, thrombotic thrombocytopenic purpura and disseminated intravascular coagulation, infections by parasites such as Plasmodium, chemical injuries from, e.g., lead poisoning, and hypersplenism.

The terms “genetic disease” or “genetic related disease” refer to a disease caused by one or more abnormalities in the genome of a subject, such as a disease that is present from birth of the subject. Genetic diseases may be heritable and may be passed down from the parents' genes. A genetic disease may also be caused by mutations or changes of the DNAs and/or RNAs of the subject. In such cases, the genetic disease will be heritable if it occurs in the germline. Exemplary genetic diseases include, but are not limited to, Aarskog-Scott syndrome, Aase syndrome, achondroplasia, acrodysostosis, addiction, adrenoleukodystrophy, albinism, ablepharon-macrostomia syndrome, alagille syndrome, alkaptonuria, alpha-1 antitrypsin deficiency, Alport's syndrome, Alzheimer's disease, asthma, autoimmune polyglandular syndrome, androgen insensitivity syndrome, Angelman syndrome, ataxia, ataxia telangiectasia, atherosclerosis, attention deficit hyperactivity disorder (ADHD), autism, baldness, Batten disease, Beckwith-Wiedemann syndrome, Best disease, bipolar disorder, brachydactyl), breast cancer, Burkitt lymphoma, chronic myeloid leukemia, Charcot-Marie-Tooth disease, Crohn's disease, cleft lip, Cockayne syndrome, Coffin Lowry syndrome, colon cancer, congenital adrenal hyperplasia, Cornelia de Lange syndrome, Costello syndrome. Cowden syndrome, craniofrontonasal dysplasia, Crigler-Najjar syndrome. Creutzfeldt-Jakob disease, cystic fibrosis, deafness, depression, diabetes, diastrophic dysplasia, DiGeorge syndrome, Down's syndrome, dyslexia, Duchenne muscular dystrophy, Dubowitz syndrome, ectodermal dysplasia Ellis-van Creveld syndrome, Ehlers-Danlos, epidermolysis bullosa, epilepsy, essential tremor, familial hypercholesterolemia, familial Mediterranean fever, fragile X syndrome, Friedreich's ataxia, Gaucher disease, glaucoma, glucose galactose malabsorption, glutaricaciduria, gyrate atrophy, Goldberg Shprintzen syndrome (velocardiofacial syndrome), Gorlin syndrome, Hailey-Hailey disease, hemihypertrophy, hemochromatosis, hemophilia, hereditary motor and sensory neuropathy (HMSN), hereditary non polyposis colorectal cancer (HNPCC), Huntington's disease, immunodeficiency with hyper-IgM, juvenile onset diabetes, Klinefelter's syndrome, Kabuki syndrome, Leigh's disease, long QT syndrome, lung cancer, malignant melanoma, manic depression, Marfan syndrome, Menkes syndrome, miscarriage, mucopolysaccharide disease, multiple endocrine neoplasia, multiple sclerosis, muscular dystrophy, myotrophic lateral sclerosis, myotonic dystrophy, neurofibromatosis, Niemann-Pick disease, Noonan syndrome, obesity, ovarian cancer, pancreatic cancer, Parkinson's disease, paroxysmal nocturnal hemoglobinuria, Pendred syndrome, peroneal muscular atrophy, phenylketonuria (PKU), polycystic kidney disease, Prader-Willi syndrome, primary biliary cirrhosis, prostate cancer, REAR syndrome, Refsum disease, retinitis pigmentosa, retinoblastoma, Rett syndrome. Sanfilippo syndrome, schizophrenia, severe combined immunodeficiency, sickle cell anemia, spina bifida, spinal muscular atrophy, spinocerebellar atrophy, sudden adult death syndrome, Tangier disease, Tay-Sachs disease, thrombocytopenia absent radius syndrome, Townes-Brocks syndrome, tuberous sclerosis, Turner syndrome, Usher syndrome, von Hippel-Lindau syndrome, Waardenburg syndrome, Weaver syndrome, Werner syndrome, Williams syndrome, Wilson's disease, xeroderma piginentosum, and Zellweger syndrome.

The term “neurological disease” refers to any disease of the nervous system, including diseases that involve the central nervous system (brain, brainstem and cerebellum), the peripheral nervous system (including cranial nerves), and the autonomic nervous system (parts of which are located in both central and peripheral nervous system). Neurodegenerative diseases refer to a type of neurological disease marked by the loss of nerve cells, including, but not limited to, Alzheimer's disease, Parkinson's disease, amyotrophic lateral sclerosis, tauopathies (including frontotemporal dementia), and Huntington's disease. Examples of neurological diseases include, but are not limited to, headache, stupor and coma, dementia, seizure, sleep disorders, trauma, infections, neoplasms, neuro-ophthalmology, movement disorders, demyelinating diseases, spinal cord disorders, and disorders of peripheral nerves, muscle and neuromuscular junctions. Addiction and mental illness, include, but are not limited to, bipolar disorder and schizophrenia, are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Arnold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (aka motor neuron disease or amyotrophic lateral sclerosis); lumbar disc disease; Lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome.

A “painful condition” includes, but is not limited to, neuropathic pain (e.g., peripheral neuropathic pain), central pain, deafferentiation pain, chronic pain (e.g., chronic nociceptive pain, and other forms of chronic pain such as post-operative pain, e.g., pain arising after hip, knee, or other replacement surgery), pre-operative pain, stimulus of nociceptive receptors (nociceptive pain), acute pain (e.g., phantom and transient acute pain), noninflammatory pain, inflammatory pain, pain associated with cancer, wound pain, burn pain, postoperative pain, pain associated with medical procedures, pain resulting from pruritus, painful bladder syndrome, pain associated with premenstrual dysphoric disorder and/or premenstrual syndrome, pain associated with chronic fatigue syndrome, pain associated with pre-term labor, pain associated with withdrawal symptoms from drug addiction, joint pain, arthritic pain (e.g., pain associated with crystalline arthritis, osteoarthritis, psoriatic arthritis, gouty arthritis, reactive arthritis, rheumatoid arthritis or Reiter's arthritis), lumbosacral pain, musculo-skeletal pain, headache, migraine, muscle ache, lower back pain, neck pain, toothache, dental/maxillofacial pain, visceral pain and the like. One or more of the painful conditions contemplated herein can comprise mixtures of various types of pain provided above and herein (e.g. nociceptive pain, inflammatory pain, neuropathic pain, etc.). In some embodiments, a particular pain can dominate. In other embodiments, the painful condition comprises two or more types of pains without one dominating. A skilled clinician can determine the dosage to achieve a therapeutically effective amount for a particular subject based on the painful condition.

In certain embodiments, the painful condition is neuropathic pain. The term “neuropathic pain” refers to pain resulting from injury to a nerve. Neuropathic pain is distinguished from nociceptive pain, which is the pain caused by acute tissue injury involving small cutaneous nerves or small nerves in muscle or connective tissue. Neuropathic pain typically is long-lasting or chronic and often develops days or months following an initial acute tissue injury. Neuropathic pain can involve persistent, spontaneous pain as well as allodynia, which is a painful response to a stimulus that normally is not painful. Neuropathic pain also can be characterized by hyperalgesia, in which there is an accentuated response to a painful stimulus that usually is trivial, such as a pin prick. Neuropathic pain conditions can develop following neuronal injury and the resulting pain may persist for months or years, even after the original injury has healed. Neuronal injury may occur in the peripheral nerves, dorsal roots, spinal cord or certain regions in the brain. Neuropathic pain conditions include, but are not limited to, diabetic neuropathy (e.g., peripheral diabetic neuropathy); sciatica; non-specific lower back pain; multiple sclerosis pain; carpal tunnel syndrome, fibromyalgia; HIV-related neuropathy; neuralgia (e.g., post-herpetic neuralgia, trigeminal neuralgia); pain resulting from physical trauma (e.g., amputation: surgery, invasive medical procedures, toxins, burns, infection), pain resulting from cancer or chemotherapy (e.g., chemotherapy-induced pain such as chemotherapy-induced peripheral neuropathy), and pain resulting from an inflammatory condition (e.g., a chronic inflammatory condition). Neuropathic pain can result from a peripheral nerve disorder such as neuroma; nerve compression; nerve crush, nerve stretch or incomplete nerve transection; mononeuropathy or polyneuropathy. Neuropathic pain can also result from a disorder such as dorsal root ganglion compression; inflammation of the spinal cord; contusion, tumor or hemisection of the spinal cord: tumors of the brainstem, thalamus or cortex; or trauma to the brainstem, thalamus or cortex.

The symptoms of neuropathic pain are heterogeneous and are often described as spontaneous shooting and lancinating pain, or ongoing, burning pain. In addition, there is pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia), or an absence of or deficit in selective sensory pathways (hypoalgesia).

In certain embodiments, the painful condition is non-inflammatory pain. The types of non-inflammatory pain include, without limitation, peripheral neuropathic pain (e.g., pain caused by a lesion or dysfunction in the peripheral nervous system), central pain (e.g., pain caused by a lesion or dysfunction of the central nervous system), deafferentation pain (e.g., pain due to loss of sensory input to the central nervous system), chronic nociceptive pain (e.g., certain types of cancer pain), noxious stimulus of nociceptive receptors (e.g., pain felt in response to tissue damage or impending tissue damage), phantom pain (e.g., pain felt in a part of the body that no longer exists, such as a limb that has been amputated), pain felt by psychiatric subjects (e.g., pain where no physical cause may exist), and wandering pain (e.g., wherein the pain repeatedly changes location in the body).

In certain embodiments, the painful condition is inflammatory pain. In certain embodiments, the painful condition (e.g., inflammatory pain) is associated with an inflammatory condition and/or an immune disorder.

The term “metabolic disorder” refers to any disorder that involves an alteration in the normal metabolism of carbohydrates, lipids, proteins, nucleic acids, or a combination thereof. A metabolic disorder is associated with either a deficiency or excess in a metabolic pathway resulting in an imbalance in metabolism of nucleic acids, proteins, lipids, and/or carbohydrates. Factors affecting metabolism include, and are not limited to, the endocrine (hormonal) control system (e.g., the insulin pathway, the enteroendocrine hormones including GLP-1, PYY, or the like), the neural control system (e.g., GLP-1 in the brain), or the like. Examples of metabolic disorders include, but are not limited to, diabetes (e.g., Type I diabetes, Type II diabetes, gestational diabetes), hyperglycemia, hyperinsulinemia, insulin resistance, and obesity.

The term “anti-inflammatory” as used herein, refers to decreasing the action or production of one or more proinflammatory cytokines (i.e., signaling molecules that are secreted from immune cells and promote inflammation) or proteins produced by nerves, neurons, glial cells, endothelial cells, fibroblasts, muscle, immune cells, or other cell types.

The term “joint” refers to an anatomical structure where two bones meet, including the ligaments that connect the bones to one another, the tendons that attach muscles to the bones, and the joint capsule, bursae, and synovium. Joints that can be treated using the methods provided herein include fixed, hinge, pivot, and ball-and-socket joints. In some embodiments, a joint is a knee, an elbow, a wrist, an ankle, a hip, a shoulder, a jaw, or a spine (including, e.g., the facet joint). In some embodiments, a joint is inflamed.

The term “joint inflammation” refers to all types of arthritis caused by inflammation. Rheumatoid arthritis and osteoarthritis are the most common forms of joint inflammation. Other types of joint inflammation include tendonitis, bursitis, inflammation of the ligament, synovitis, gout, facet syndrome, and systemic lupus erythematosus. In certain embodiments, the joint inflammation is osteoarthritis.

The terms “infectious disease” and “infectious disorder” refer to diseases and disorders caused by microorganisms, for example bacteria, viruses, fungi, or parasite. Exemplary infectious diseases include, but are not limited to, Acute Flaccid Myelitis (AFM), anaplasmosis, anthrax, babesiosis, botulism, brucellosis, campylobacteriosis, carbapenem-resistant infection (CRE/CRPA), chancroid, chikungunya virus infection, chlamydia, ciguatera (Harmful Algae Blooms (HABs)), Clostridium difficile infection, Clostridium perfringens (epsilon toxin), coccidioidomycosis fungal infection (valley fever), COVID-19, Creutzfeldt-Jakob disease, transmissible spongiform encephalopathy (CJD), cryptosporidiosis, cyclosporiasis, dengue fever, diphtheria, E. coli infection, Shiga toxin-producing (STEC), eastern equine encephalitis, Ebola, ehrlichiosis, encephalitis, arboviral or parainfectious, enterovirus infection, non-polio enterovirus, enterovirus infection, D68 (EV-D68), giardiasis (giardia), glanders, gonococcal infection (gonorrhea), granuloma inguinale, haemophilus influenza disease, hantavirus pulmonary syndrome, hemolytic uremic syndrome, hepatitis A, hepatitis B, hepatitis C, hepatitis D, hepatitis E, herpes, herpes zoster, shingles, histoplasmosis infection, human immunodeficiency virus, AIDS, human papillomavirus, influenza, lead poisoning, legionellosis (Legionnaires Disease), leprosy (Hansens Disease), leptospirosis, listeriosis (Listeria), Lyme disease, lymphogranuloma venereum infection (LGV), malaria, measles, melioidosis, meningitis, viral (Meningitis, viral), meningococcal disease, bacterial (Meningitis, bacterial), middle east respiratory syndrome coronavirus (MERS-CoV), multisystem inflammatory syndrome in children (MIS-C), mumps, norovirus, paralytic shellfish poisoning, pediculosis, lice, pelvic inflammatory disease, pertussis, plague (bubonic, septicemic, pneumonic), pneumonia, poliomyelitis (Polio), Powassan virus, psittacosis (Parrot Fever), pthiriasis, pustular rash diseases (small pox, monkeypox, cowpox), Q-Fever, rabies, ricin poisoning, rickettsiosis (Rocky Mountain Spotted Fever), rubella, Salmonella, scabies, scombroid, septic shock (Sepsis), severe acute respiratory syndrome (SARS), shigellosis gastroenteritis (Shigella), smallpox, staphylococcal infections (MRSA, staph food poisoning, VISA, VRSA), streptococcal Disease (strep A, strep B), streptococcal toxic-shock syndrome, syphilis, tetanus infection, trichomoniasis, trichonosis infection, tuberculosis, tularemia, typhoid fever, typhus, vaginosis, vaping-associated lung injury, varicella, Vibrio cholerae (cholera), vibriosis (Vibrio), viral hemorrhagic fever (Ebola, Lassa, Marburg), West Nile Virus, yellow fever, yersenia, or zika virus infection (Zika).

The terms “cardiovascular disease” and “cardiovascular disorder” refer to diseases and disorders that affect the hear or blood vessels, Exemplary cardiovascular diseases include, but are not limited to, angina, arrhythmia, congenital heart disease, coronary artery disease, heart attack, heart failure, dilated cardiomyopathy, hypertrophic cardiomyopathy, mitral regurgitation, mitral valve prolapse, pulmonary stenosis, aortic stenosis, atrial fibrillation, rheumatic heart disease, radiation heart disease, peripheral artery disease, aneurysm, atherosclerosis, renal artery disease, Raynaud's disease, peripheral venous disease, ischemic stroke, venous blood clots, blood clotting disorders, or Buerger's disease.

The terms “cerebrovascular disease” and “cerebrovascular disorder” refer to diseases and disorders that affect blood flow and blood vessels in the brain. The diseases and disorders may be due to stenosis, thrombosis, embolism, or hemorrhage, Exemplary cerebrovascular diseases include, but are not limited to, aneurysm, arteriovenous malformation (AVM), cerebral cavernous malformation (CCM), arteriovenous fistula (AVF), carotid-cavernous fistula, carotid stenosis, transient ischemic attack (TIA), or stroke.

The terms “pulmonary disease” or “pulmonary disorder” refer to diseases and disorders relating to the lungs, Exemplary pulmonary diseases include, but are not limited to, asbestosis, asthma, bronchiectasis, bronchitis, chronic cough, chronic obstructive pulmonary disease (COPD), common cold, COVID-19, croup, cystic fibrosis, hantavirus, influenza, idiopathic pulmonary fibrosis, lung cancer, pandemic flu, pertussis, pleurisy, pneumonia, pulmonary edema, pulmonary Embolism, pulmonary fibrosis, pulmonary Hypertension, respiratory syncytial virus (RSV), sarcoidosis, sleep apnea, spirometry, sudden infant death syndrome (SIDS), or tuberculosis.

The terms “dermatological disease” or “dermatological disorder” refers to diseases and disorders relating to the skin, Exemplary dermatological diseases include, but are not limited to, acanthoma fissuratum, acanthosis nigricans, accessory tragus, acne, acne excoriée, acne keloidalis nuchae, acquired digital fibrokeratoma, acrochordons, acrodermatitis enteropathica, acropustulosis of infancy, actinic cheilitis, actinic keratosis, actinic purpura, dolorosa (Dercum's disease), albinism, alkaptonuria, allergic contact dermatitis, alopecia areata, alopecia mucinosa, androgenetic alopecia, anetoderma, angioedema, angiofibroma, angiokeratoma, angiomas, angular cheilitis, aphthous ulcer, aplasia cutis congenita, ashy dermatosis, asteatotic eczema, atopic dermatitis, atrophie blanche, atrophoderma of pasini and pierini, atypical moles, balanitis, basal cell carcinoma, basal cell nevus syndrome, Becker's nevus, bee and wasp stings, black hairy tongue, Blaschko's lines, blue nevus, boils, Bowen's disease, Bowenoid papulosis, brachioradial pruritus, bullous pemphigoid, Buruli ulcer, calcipotriene, canker sore, capsaicin, carbuncle, cellulitis, central centrifugal cicatricial alopecia, chancroid, cherry angioma, chicken pox, chondrodermatitis nodularis helicis, condyloma acuminata, confluent and reticulated papillomatosis, congenital adrenal hyperplasia, contact dermatitis, Cowden syndrome, cutaneous T cell lymphoma, cutis marmorata, cysts, dandruff, Darier disease, dermal fillers, dermatitis herpetiformis, dermatofibroma, dermatofibrosarcoma protuberans, dermatographism, dermatomyositis, dermatosis papulosa nigra, diaper dermatitis, digital mucous cyst, discoid lupus erythematosus, disseminated superficial actinic porokeratosis, DRESS syndrome, dry skin, dyshidrotic dermatitis, dysplastic moles, eczema, Ehlers-Danlos syndrome, elastosis perforans serpiginosa, epidermal cyst, epidermal nevus, epidermolysis bullosa, epidermolysis bullosa acquisita, erosive pustular dermatosis, erysipelas, erythema ab igne, erythema annulare centrifugum, erythema dyschromicum perstans, erythema infectiosum, erythema multiforme, erythema nodosum, erythema toxicum neonatomm, erythrasma, erythromelalgia, etanercept, exanthem subitum, Fabry disease, Favre-Racouchot syndrome, female pattern hair loss, fifth disease (also referred to as erythema infectiosum), fire ant bites, fish tank granuloma, flea bites, focal dermal hypoplasia, folliculitis, Fordyce spots, Fox-Fordyce disease, frostbite, fungal infections, furuncle, geographic tongue, Gianotti-Crosti syndrome, glomus tumor, Gorlin syndrome, granuloma annulare, granuloma inguinale, green nail syndrome, Grover's disease, habit tic nail deformity, Hailey-Hailey disease, hair loss, hair removal, hair transplantation, halo moles, hand rashes, hand foot and mouth disease, head lice, hemangiomas, Henoch-Schonlein purpura, hereditary hemorrhagic telangiectasia, herpes, herpes zoster, hidradenitis suppurativa, hidrocystoma, hirsutism, hives, hot tub folliculitis, hyperhidrosis, hyperpigmentation, hypersensitivity vasculitis, hypomelanosis of ito, ichthyosis, idiopathic guttate hypomelanosis, impetigo, incontinentia pigmenti, ingenol mebutate, intertrigo, itching, Jessner lymphocytic infiltrate, jiggers, juvenile plantar dermatosis, juvenile xanthogranuloma, Kaposi's sarcoma, Kawasaki's disease, keloids and hypertrophic scars, keratoacanthoma, keratosis follicularis spinulosa decalvans, keratosis pilaris, Kyrle's disease, leiomyoma, leishmaniasis, lentigines, lentigo maligna, leprosy, leukocytoclastic vasculitis, leukoplakia, lice, lichen amyloidosis, lichen nitidus, lichen planus, lichen sclerosus, lichen simplex chronicus, lichen spinulosus, lichen striatus, linear IgA bullous dermatosis, lipodermatosclerosis, lipoma, loose anagen syndrome, Lyme disease, lymphangioma circumscriptum, lymphogranuloma venereum, majocchi granuloma, mastocytoma, mastocytosis, measles, melanoma, acral lentiginous, melanoma in situ, melanonychia, melasma, Melkersson-Rosenthal syndrome, meralgia paresthetica, Merkel cell carcinoma, metastatic skin cancer, methotrexate, milia, miliaria, moles, molluscum contagiosum, Mongolian spot, monilethrix, morphea, mucocele, muir-torre syndrome, mycophenolate mofetil, mycosis fungoides, myiasis, nail fungus, necrobiosis lipoidica diabeticorum, neurofibromatosis, nevoid basal cell carcinoma syndrome, nevus achromicus, nevus anemicus, nevus flammeus, nevus sebaceus, nevus spilus, nevus of ota and ito, notalgia paresthetica, nummular eczema, ochronosis, onycholysis, onychomycosis, onychophagia, orange palpebral spots, paederus dermatitis, Paget's disease, palmoplantar pustulosis, panniculitis, parapsoriasis, paronychia nail infection, pediculosis, pellagra, pemphigus, pemphigoid gestationis, perioral dermatitis, perleche, Peutz-Jeghers syndrome, phytophotodermatitis, pilar cyst, pilomatricoma, pimecrolimus, pitted keratolysis, Pityriasis alba, Pityriasis lichenoides, Pityriasis rosea, Pityriasis rubra pilaris, pityrosporum folliculitis, poikiloderma of civatte, poison ivy dermatitis, polymorphous light eruption, porokeratosis of mibelli, Porphyria cutanea tarda, postherpetic neuralgia, pressure ulcers, progressive pigmentary purpura, prurigo nodularis, prurigo pigmentosa, pruritic urticarial papules and plaques of pregnancy, pseudofolliculitis barbae, pseudoxanthoma elasticum, psoriasis, punctate palmoplantar keratoderma, pyoderma gangrenosum, pyogenic granuloma, red scrotum syndrome, rheumatoid nodules, rocky mountain spotted fever, rosacea, Roseola infantum, sarcoidosis, scabies, scarlet fever, Schamberg's disease, scleroderma, sebaceous cyst, sebaceous hyperplasia, seborrheic dermatitis, seborrheic keratoses, shingles, skin tags, spider angioma, spider bites, spitz nevus, sporotrichosis, squamous cell carcinoma, staphylococcal scalded skin syndrome, stasis dermatitis, steatocystoma multiplex, Stevens Johnson syndrome, stretch marks, striae, Sturge-Weber syndrome, subacute cutaneous lupus erythematosus, subungual hematoma, sweet's syndrome, swimmer's itch, syphilis, syringoma, systemic lupus erythematosus, systemic sclerosis, elangiectasia Macularis eruptiva perstans, telogen effluvium hair loss, tinea, tinea incognito, Tinea versicolor, toxic epidermal necrolysis, transient neonatal pustular melanosis, tretinoin, trichilemmal cyst, trichotillomania, tuberous sclerosis, twenty nail dystrophy, urticaria, urticaria pigmentosum, varicella, vitiligo, warts, washboard nail, xanthelasma, xeroderma pigmentosum, xerosis, or xerotic eczema.

The terms “bone diseases” and “bone disorders” refer to diseases and disorders affecting bones, Exemplary bone diseases include, but are not limited to, bone spurs, bone tumor, chondroblastoma chondromyxoid fibroma, enchondroma extra-abdominal desmoid tumors, fibrous dysplasia, hypophosphatasia, Klippel-Feil Syndrome osteochondritis dissecans (OCD) osteochondroma, or osteoid osteoma.

The terms “hormonal diseases” and “hormonal disorders” refer to diseases and disorders regulated or related to hormones and/or the endocrine system, Exemplary hormonal diseases include, but are not limited to, acromegaly, Addison's Disease, adrenal cancer, adrenal disorders, anaplastic thyroid cancer, Cushing's Syndrome, De Quervain's thyroiditis, diabetes, follicular thyroid cancer, gestational diabetes, goiters, Graves' Disease, growth disorders, growth hormone deficiency, Hashimoto's thyroiditis, heart disease, Hurthle cell thyroid cancer, hyperglycemia, hyperparathyroidism, hyperthyroidism, hypoglycemia, hypoparathyroidism, hypothyroidism, low testosterone, medullary thyroid cancer, MEN 1, MEN 2A, MEN 2B, menopause, metabolic syndrome, obesity, osteoporosis, papillary thyroid cancer, parathyroid diseases, pheochromocytoma, pituitary disorders, pituitary tumors, polycystic ovary syndrome, prediabetes, reproduction, silent thyroiditis, thyroid cancer, thyroid diseases, thyroid nodules, thyroiditis, turner syndrome, Type 1 diabetes, or Type 2 diabetes.

A “protein,” “peptide,” or “polypeptide” comprises a polymer of amino acid residues linked together by peptide bonds. The term refers to proteins, polypeptides, and peptides of any size, structure, or function, Typically, a protein will be at least three amino acids long, A protein may refer to an individual protein or a collection of proteins. Inventive proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification, A protein may also be a single molecule or may be a multi-molecular complex, A protein may be a fragment of a naturally occurring protein or peptide, A protein may be naturally occurring, recombinant, synthetic, or any combination of these.

The term “gene” refers to a nucleic acid fragment that expresses a protein, including regulatory sequences preceding (5′ non-coding sequences) and following (3′ non-coding sequences) the coding sequence, “Native gene” refers to a gene as found in nature with its own regulatory sequences, “Chimeric gene” or “chimeric construct” refers to any gene or a construct, not a native gene, comprising regulatory and coding sequences that are not found together in nature. Accordingly, a chimeric gene or chimeric construct may comprise regulatory sequences and coding sequences that are derived from different sources, or regulatory sequences and coding sequences derived from the same source, but arranged in a manner different than that found in nature, “Endogenous gene” refers to a native gene in its natural location in the genome of an organism, A “foreign” gene refers to a gene not normally found in the host organism, but which is introduced into the host organism by gene transfer, Foreign genes can comprise native genes inserted into a non-native organism, or chimeric genes.

A “transgene” is a gene that has been introduced into the genome by a transformation procedure, A transgene (e.g., a coding region of a transgene) may encode any therapeutic agent, including, but not limited to a therapeutic protein, an antibody or fragment thereof, a bispecific antibody or fragment thereof, antigen-binding fragments, a nucleic acid molecule-based therapeutic (e.g., an siRNA, a microRNA, or an oligonucleotide), genome editing components (e.g., CRISPR/Cas9 based proteins and protein fusion and guide RNA molecules), and complexes (e.g., nucleoprotein complexes).

A coding region of a transgene may be naturally-occurring, and may in some embodiments comprise no nucleic acid modifications, relative to the coding region of a wild-type gene. In some embodiments, a coding region of a transgene may be synthetic. The coding region of a transgene may be considered synthetic if it undergoes one or more nucleic acid modifications, relative to the coding region of a wild-type gene, A nucleic acid modification may be a substitution or deletion of one or more nucleotides that form the nucleic acid sequence of the coding region of the transgene. In some embodiments, the modification comprises disrupting or deleting a native start codon located at the 5 end of the coding region of the transgene. In some embodiments, the modification comprises the insertion of an alternatively-spliced exon into the coding region of the transgene.

The terms “polynucleotide”, “nucleotide sequence”, “nucleic acid”, “nucleic acid molecule”, “nucleic acid sequence”, and “oligonucleotide” refer to a series of nucleotide bases (also called “nucleotides”) in DNA and RNA, and mean any chain of two or more nucleotides. The polynucleotides can be chimeric mixtures or derivatives or modified versions thereof, single-stranded or double-stranded. The oligonucleotide can be modified at the base moiety, sugar moiety, or phosphate backbone, for example, to improve stability of the molecule, its hybridization parameters, etc. The antisense oligonucleotide may comprise a modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, I-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, a thio-guanine, and 2,6-diaminopurine, A nucleotide sequence typically carries genetic information, including the information used by cellular machinery to make proteins and enzymes. These terms include double- or single-stranded genomic and cDNA, RNA, any synthetic and genetically manipulated polynucleotide, and both sense and antisense polynucleotides. This includes single- and double-stranded molecules, i.e., DNA-DNA, DNA-RNA and RNA-RNA hybrids, as well as “protein nucleic acids” (PNAs) formed by conjugating bases to an amino acid backbone. This also includes nucleic acids containing carbohydrate or lipids, Exemplary DNAs include single-stranded DNA (ssDNA), double-stranded DNA (dsDNA), plasmid DNA (pDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), provirus, lysogen, repetitive DNA, satellite DNA, and viral DNA, Exemplary RNAs include single-stranded RNA (ssRNA), double-stranded RNA (dsRNA), small interfering RNA (siRNA), messenger RNA (mRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, and viral satellite RNA. In some embodiments, the DNA is plasmid DNA (pDNA).

Polynucleotides described herein may be synthesized by standard methods known in the art, e.g., by use of an automated DNA synthesizer (such as those that are commercially available from Biosearch, Applied Biosystems, etc). As examples, phosphorothioate oligonucleotides may be synthesized by the method of Stein et al., Nucl. Acids Res., 16, 3209, (1988), methylphosphonate oligonucleotides can be prepared by use of controlled pore glass polymer supports (Sarin et al., Proc. Natl. Acad. Sci. U.S.A, 85, 7448-7451, (1988)), A number of methods have been developed for delivering antisense DNA or RNA to cells, e.g., antisense molecules can be injected directly into the tissue site, or modified antisense molecules, designed to target the desired cells (antisense linked to peptides or antibodies that specifically bind receptors or antigens expressed on the target cell surface) can be administered systemically. Alternatively, RNA molecules may be generated by in vitro and in vivo transcription of DNA sequences encoding the antisense RNA molecule, Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. However, it is often difficult to achieve intracellular concentrations of the antisense sufficient to suppress translation of endogenous mRNAs. Therefore, a preferred approach utilizes a recombinant DNA construct in which the antisense oligonucleotide is placed under the control of a strong promoter. The use of such a construct to transfect target cells in the patient will result in the transcription of sufficient amounts of single stranded RNAs that will form complementary base pairs with the endogenous target gene transcripts and thereby prevent translation of the target gene mRNA, For example, a vector can be introduced in vivo such that it is taken up by a cell and directs the transcription of an antisense RNA, Such a vector can remain episomal or become chromosomally integrated, as long as it can be transcribed to produce the desired antisense RNA, Such vectors can be constructed by recombinant DNA technology methods standard in the art, Vectors can be plasmid, viral, or others known in the art, used for replication and expression in mammalian cells, Expression of the sequence encoding the antisense RNA can be by any promoter known in the art to act in mammalian, preferably human, cells, Such promoters can be inducible or constitutive, Any type of plasmid, cosmid, yeast artificial chromosome, or viral vector can be used to prepare the recombinant DNA construct that can be introduced directly into the tissue site.

The polynucleotides may be flanked by natural regulatory (expression control) sequences or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art, Non-limiting examples of such modifications include methylation, “caps”, substitution of one or more of the naturally occurring nucleotides with an analog, and internucleotide modifications, such as, for example, those with uncharged linkages (e.g., methyl phosphonates, phosphotriesters, phosphoroamidates, carbamates, etc,) and with charged linkages (e.g., phosphorothioates, phosphorodithioates, etc,), Polynucleotides may contain one or more additional covalently linked moieties, such as, for example, proteins (e.g., nucleases, toxins, antibodies, signal peptides, poly-L-lysine, etc,), intercalators (e.g., acridine, psoralen, etc,), chelators (e.g., metals, radioactive metals, iron, oxidative metals, etc,), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly, Exemplary labels include radioisotopes, fluorescent molecules, isotopes (e.g., radioactive isotopes), biotin, and the like. In some embodiments, the polynucleotides are flanked by inverted terminal repeat (ITR) sequences. In some embodiments, an ITR sequence is an adeno-associated virus (AAV) ITR sequence. In some embodiments, the ITR sequence is an AAV-2 ITR. In some embodiments, AVV-2 ITRs flake the transcription cassette.

A “recombinant nucleic acid molecule” is a nucleic acid molecule that has undergone a molecular biological manipulation, i.e., non-naturally occurring nucleic acid molecule or genetically engineered nucleic acid molecule. Furthermore, the term “recombinant DNA molecule” refers to a nucleic acid sequence which is not naturally occurring, or can be made by the artificial combination of two otherwise separated segments of nucleic acid sequence, i.e., by ligating together pieces of DNA that are not normally continuous, By “recombinantly produced” is meant artificial combination often accomplished by either chemical synthesis means, or by the artificial manipulation of isolated segments of nucleic acids, e.g., by genetic engineering techniques using restriction enzymes, ligases, and similar recombinant techniques as described by, for example, Sambrook et al., Molecular Cloning, second edition, Cold Spring Harbor Laboratory, Plainview, N.Y.; (1989), or Ausubel et al., Current Protocols in Molecular Biology, Current Protocols (1989), and DNA Cloning: A Practical Approach, Volumes I and II (ed, D, N, Glover) IREL Press, Oxford, (1985); each of which is incorporated herein by reference.

Such manipulation may be done to replace a codon with a redundant codon encoding the same or a conservative amino acid, while typically introducing or removing a sequence recognition site. Alternatively, it may be performed to join together nucleic acid segments of desired functions to generate a single genetic entity comprising a desired combination of functions not found in nature, Restriction enzyme recognition sites are often the target of such artificial manipulations, but other site specific targets, e.g., promoters, DNA replication sites, regulation sequences, control sequences, open reading frames, or other useful features may be incorporated by design.

The term “pDNA,” “plasmid DNA,” or “plasmid” refers to a small DNA molecule that is physically separate from, and can replicate independently of, chromosomal DNA within a cell, Plasmids can be found in all three major domains: Archaea, Bacteria, and Eukarya. In nature, plasmids carry genes that may benefit survival of the subject (e.g., antibiotic resistance) and can frequently be transmitted from one bacterium to another (even of another species) via horizontal gene transfer, Artificial plasmids are widely used as vectors in molecular cloning, serving to drive the replication of recombinant DNA sequences within host subjects, Plasmid sizes may vary from 1 to over 1,000 kbp. Plasmids are considered replicons, capable of replicating autonomously within a suitable host.

The term “DNA minicircle” refers to a small (˜4 kb) circular plasmid derivative that has been freed from all prokaryotic vector parts (e.g., no longer contains a bacterial plasmid backbone comprising antibiotic resistance markers and/or bacterial origins of replication), Methods of producing DNA minicircles are well-known in the art. For example, a parental plasmid that comprises a bacterial backbone and the eukaryotic inserts, including the transgene to be expressed, may be produced in a specialized E, coli strain that expresses a site-specific recombinase protein, Recombination sites flank the eukaryotic inserts in the parental plasmid, so that when the activity of the recombinase protein is induced by methods such as, but not limited to, arabinose induction, glucose induction, etc., the bacterial backbone is excised from the eukaryotic insert, resulting in a eukaryotic DNA minicircle and a bacterial plasmid.

The term DNA “control sequences” refers collectively to promoter sequences, polyadenylation signals, transcription termination sequences, upstream regulatory domains, origins of replication, internal ribosome entry sites, enhancers, and the like, which collectively provide for the replication, transcription, and translation of a coding sequence in a recipient cell. The vectors provided herein contain one or more control sequence, Notably, not all types of control sequences need to be present, as long as the selected coding sequence is capable of being replicated, transcribed, and translated in an appropriate host cell.

The term “nuclear targeting sequence” refers to a nucleic acid sequence that functions to improve the expression efficiency of an anti-inflammatory cytokine in a cell.

“Operably linked” refers to an arrangement of elements where the components so described are configured so as to perform their usual function, Thus, control sequences operably linked to a coding sequence are capable of effecting the expression of the coding sequence. The control sequences need not be contiguous with the coding sequence so long as they function to direct the expression thereof, Thus, for example, intervening untranslated yet transcribed sequences can be present between a promoter sequence and the coding sequence, and the promoter sequence can still be considered “operably linked” to the coding sequence.

The term “promoter” is used herein in its ordinary sense to refer to a nucleotide region comprising a DNA regulatory sequence, wherein the regulatory sequence is derived from a gene that is capable of binding RNA polymerase and initiating transcription of a downstream (3′-direction) coding sequence, Transcription promoters can include “inducible promoters” (where expression of a polynucleotide sequence operably linked to the promoter is induced by an analyte, cofactor, regulatory protein, etc,), “repressible promoters” (where expression of a polynucleotide sequence operably linked to the promoter is induced by an analyte, cofactor, regulatory protein, etc,), and “constitutive promoters,” In some embodiments, promoters may be chicken or human β-actin promoters, cytomegalovirus immediate early promoters, glyceraldehyde 3-phosphate dehydrogenase (GAPDH) promoters, elongation factor 1α (eF1α) promoters, GFAP promoters, murine leukemia virus (MLV) promoters, herpes simplex virus thymidine kinase (TK) promoters, and woodchuck hepatitis virus post-transcriptional regulatory element (WPRE) promoters. In certain embodiments, a promoter is a cytomegalovirus immediate early promoter. In some embodiments, the promoter (e.g., constitutive promoter) is selected from the group consisting of ubiquitin promoters, CMV promoters, β-actin promoters, histone H4 promoters, EF-1α promoters, PGK gene promoters, promoter elements controlled by RNA polymerase II, promoter elements controlled by RNA polymerase I, promoter elements controlled by RNA polymerase III, U6 promoters (e.g., U6-1 promoters, U6-8 promoters, U6-9 promoters), H1 promoter, 7SL promoter, human Y promoters (e.g., hY1 promoters, hY3 promoters, hY4 promoters, and hY5 promoters), human MRP-7-2 promoter, adenovirus VA 1 promoter, human tRNA promoters, and 5s ribosomal RNA promoters.

For the purpose of describing the relative position of nucleotide sequences in a particular nucleic acid molecule throughout the instant application, such as when a particular nucleotide sequence is described as being situated “upstream,” “downstream,” “3 prime (3′)” or “5 prime (5′)” relative to another sequence, it is to be understood that it is the position of the sequences in the “sense” or “coding” strand of a DNA molecule that is being referred to, as is conventional in the art.

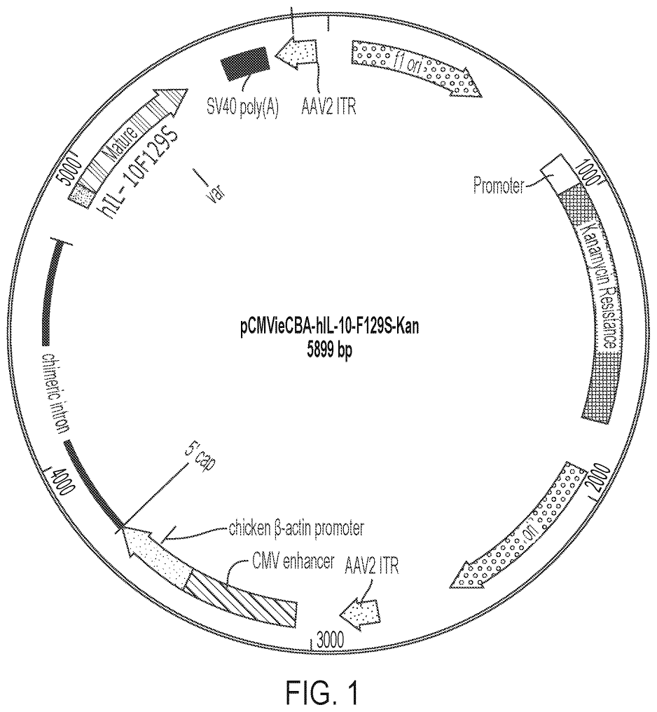

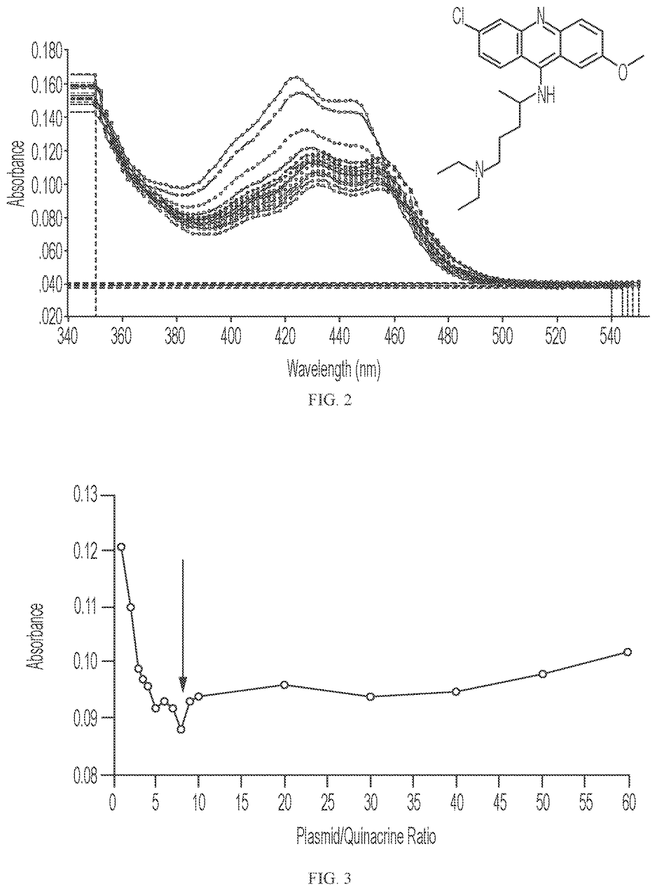

The term “XT-150” refers to a circular vector of SEQ ID NO: 2 as provided herein, and as shown in FIG. 1, which encodes IL-10^{F129S}.

The term “XT-151” refers to a circular vector of SEQ ID NO: 28 as provided herein which encodes IL-10^{F129S} and IL-10R1.

The term “cytokine” as used herein refers to a polypeptide(s)/glycoprotein(s) derived from a natural lymphokine (i.e., cytokines made by lymphocytes), monokine (cytokines made by monocytes), chemokine (i.e., cytokines with chemotactic activities), an interleukin (i.e., cytokines made by one leukocyte and acting on other leukocytes), or modification thereof, Examples of cytokines include, but are not limited to, interleukin (IL) (e.g., such as IL-1α, IL-1β, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-37, IL-38, IL-39, and IL-40; interferon (IFN) (e.g., IFN-α, IFN-β, and IFN-γ); tumor necrosis factor (TNF) (e.g., TNF-α, TNF-β, LT-β CD154, 4-1BB1, APRIL, CD70, CD153, CD178, TRAIL, TWEAK, TRANCE, TALL-1); transforming growth factor (TGF) (e.g., TGF-α and TGF-β (e.g., TGF-β1, TGF-β2, TGF-β3)): colony stimulating factor (CSF) (e.g., granulocyte-colony stimulating factor (G-CSF), granulocyte-macrophage-colony 10 stimulating factor (GM-CSF), macrophage-colony stimulating factor (M-CSF), macrophage stimulating factor (MSP)); erythropoietin (EPO); stem cell factor (SCF); monocyte chemotactic and activating factor (MCAF); oncostatin M (OSM); growth factor (GF) (e.g., epidermal growth factor (EGF), fibroblast growth factor (FGF), insulin like growth 15 factor (IGF), nerve growth factor (NGF), Brain-derived neurotrophic factor (BONE), platelet derived growth factor (PDGF), vascular endothelial growth factor (VEGF), hepatocyte growth factor (HGF), keratinocyte growth factor (KGF)); thrombopoietin (TPO); bone morphogenic protein 20 (BMP); LIF; kit ligand (KL): MPO (myeloperoxidase); CRP (C-reactive protein); COX (cyclooxygenase) (e.g., COX-1, COX-2 and COX-3); NOS (Nitric oxide synthase) (e.g., NOS-1, NOS-2 and NOS-3); cytokine receptor antagonists (e.g., such as IL-1Ra); and modified variations thereof, Cytokines also includes chemokines which are cytokines that induce chemotaxis, CXC chemokines include neutrophil-activating protein-2 (NAP-2) and melanoma growth stimulatory activity 5 protein (MGSA), CC chemokines, include RANTES, Macrophage inflammatory protein (MIP) (e.g., MIP-1α, MIP-1β), keratinocyte-derived chemokine (KC), monocyte 10 chemotactic proteins (MCP-1, MCP-2, MCP-3, MCP-4, and MCP-5), and eotaxins (e.g., exotaxin-1, exotaxin-2), Further examples include lymphotactin-1, lymphotactin-2, and fractalkine.

As used herein, “glycoprotein” refers to proteins which contain glycans (oligosaccharides) covalently attached to amino acid side-chains.

The terms “IL-10 receptor type 1” or “IL-10R1” (NCBI Reference Sequence for human: NP_001549.2) refer to a glycoprotein with a single transmembrane domain expressed on the surface of certain cell types, chiefly antigen-presenting cells, IL-10R1 binds IL-10 and is essential for the biological activity of IL-10. The binding of IL-10 to its cell surface receptors activates the JAK-STAT signal transduction pathway, Following the ligand-receptor interaction, Jak1 (associated with IL-10R1) and Tyk2 (associated with IL-10R2), members of the receptor-associated Janus tyrosine kinases (JAK) family, are phosphorylated, IL-10R1 is subject to proteasomal degradation after ubiquitination by SOCS3.

The term “transcription factor” refers to a DNA-binding protein that regulates transcription of DNA into RNA, for example, by activation or repression of transcription, Some transcription factors effect regulation of transcription alone, while others act in concert with other proteins, Some transcription factor can both activate and repress transcription under certain conditions. In general, transcription factors bind a specific target sequence or sequences highly similar to a specific consensus sequence in a regulatory region of a target gene, Transcription factors may regulate transcription of a target gene alone or in a complex with other molecules, Examples of transcription factors include, but are not limited to, Sp1, NF1, CCAAT, GATA, HNF, PIT-1, MyoD, Myf5, Hox, Winged Helix, SREBP, p53, CREB, AP-1, Mef2, STAT, R-SMAD, NF-κB, Notch, TUBBY, and NFAT.

The term “antibody” refers to a full-length (i.e., naturally occurring or formed by normal immunoglobulin gene fragment recombinatorial processes) immunoglobulin molecule (e.g., an IgG antibody) or an immunologically active (i.e., specifically binding) portion of an immunoglobulin molecule, like an antibody fragment. An antibody is a glycoprotein comprising at least two heavy (H) chains and two light (L) chains inter-connected by disulfide bonds, Each heavy chain is comprised of a heavy chain variable region (abbreviated herein as V_{H}) and a heavy chain constant region. The heavy chain constant region is comprised of three subdomains, C_{H1}, C_{H2} and C_{H3}, Each light chain is comprised of a light chain variable region (abbreviated herein as V_{L}) and a light chain constant region. The light chain constant region is comprised of one subdomain, C_{L}. The V_{H} and V_{L} regions can be further subdivided into regions of hypervariability, termed complementarity determining regions (CDR), interspersed with regions that are more conserved, termed framework regions (FR), Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

### DETAILED DESCRIPTION OF THE INVENTION

Disclosed herein are compounds (e.g., compound of Formula (I)), compositions (e.g., comprising compounds, comprising compounds and DNA (e.g., double-stranded DNA (e.g., plasmid DNA)), kits, methods, and uses. The disclosure seeks to improve uptake of DNA (e.g., double-stranded DNA (e.g., plasmid DNA)) into cells (e.g., targeted cells) by using compounds of Formula (I) which both interact with DNA (i.e., through the DNA-interacting moiety, R²) and comprises a sugar, which directs the DNA to certain cells (e.g., cells expressing receptors for the sugar),

### Compounds

A compound of Formula (I):

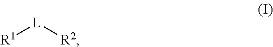

or a pharmaceutically acceptable salt thereof, wherein:
R¹ is substituted or unsubstituted sugar, substituted or unsubstituted amino sugar, or substituted or unsubstituted deoxy sugar;
L is substituted C₁₋₂₀ alkylene or unsubstituted C₁₋₂₀ alkylene, wherein:
   optionally one or more backbone carbon atoms in the substituted C₁₋₂₀ alkylene or unsubstituted C₁₋₂₀ alkylene are independently replaced with —NR^{A}—, —O—, —C(═O)—, —S—, substituted or unsubstituted carbocyclylene, substituted or unsubstituted heterocyclylene, substituted or unsubstituted arylene, or substituted or unsubstituted heteroarylene:
R² is a DNA-interacting agent; and
R^{A} is hydrogen, substituted or unsubstituted C₁₋₆ alkyl, —C(═O)(C₁₋₆ alkyl), or a nitrogen protecting group;
provided that the compound is not of the formula:

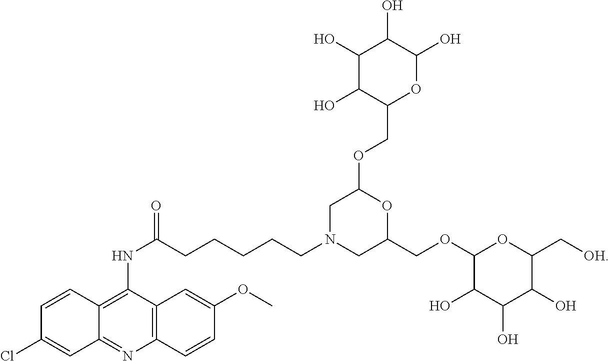 [SMILES: COc1ccc2nc3cc(Cl)ccc3c(NC(=O)CCCCCN3CC(COC4OC(CO)C(O)C(O)C4O)OC(OCC4OC(O)C(O)C(O)C4O)C3)c2c1]

In some embodiments, the compound of Formula (I) is not of the formula:

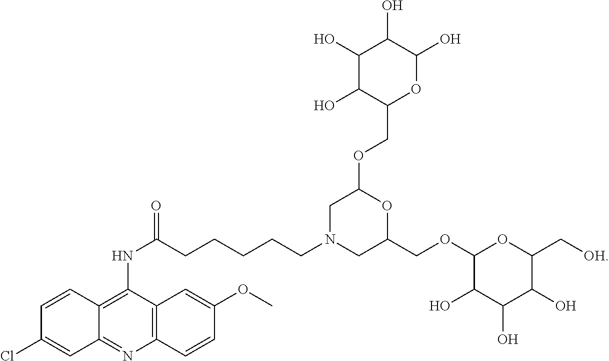 [SMILES: COc1ccc2nc3cc(Cl)ccc3c(NC(=O)CCCCCN3CC(COC4OC(CO)C(O)C(O)C4O)OC(OCC4OC(O)C(O)C(O)C4O)C3)c2c1]

In some embodiments, the compound of Formula (I) is of Formula (I-A):

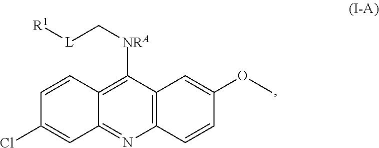

or a pharmaceutically acceptable salt thereof.

In some embodiments, the compound of Formula (I) is of Formula (I-B):

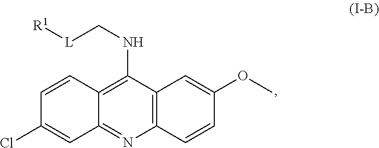

or a pharmaceutically acceptable salt thereof.

In some embodiments, the compound of Formula (I) is of the formula:

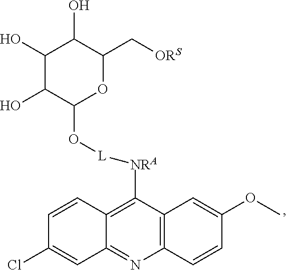

or a pharmaceutically acceptable salt thereof. In some embodiments, R^{S} is —H, —SO₃H, or —PO₃H₂. In some embodiments, R^{S} is —H or —SO₃H.

In some embodiments, the compound of Formula (I) is of the formula:

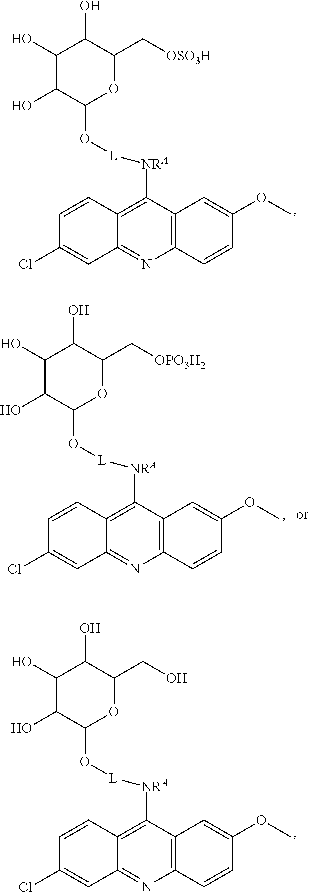

or a pharmaceutically acceptable salt thereof.

In some embodiments, the compound of Formula (I) is of the formula:

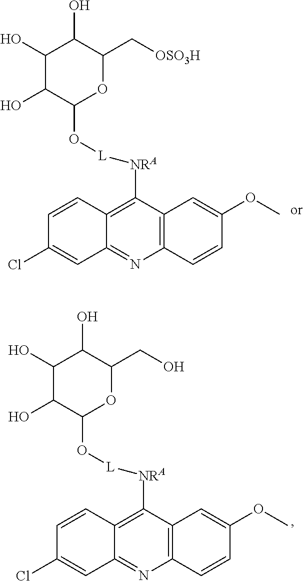

or a pharmaceutically acceptable salt thereof.

In some embodiments, the compound of Formula (I) is of the formula:

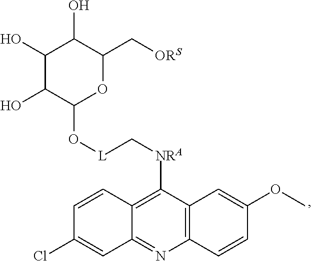

or a pharmaceutically acceptable salt thereof. In some embodiments, R^{S} is —H, —SO₃H, or —PO₃H₂. In some embodiments, R^{S} is —H or —SO₃H.

In some embodiments, the compound of Formula (I) is of the formula:

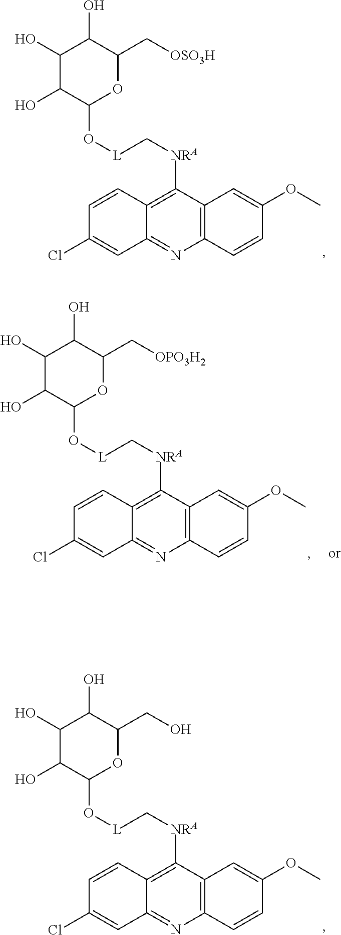

or a pharmaceutically acceptable salt thereof.

In some embodiments, the compound of Formula (I) is of the formula:

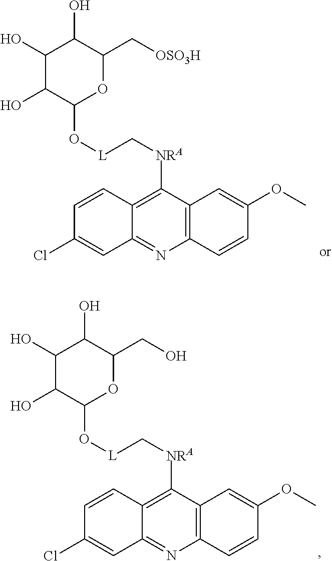

or a pharmaceutically acceptable salt thereof.

In some embodiments, the compound of Formula (I) is of the formula:

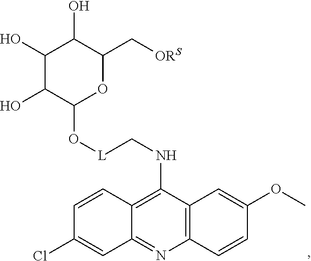

or a pharmaceutically acceptable salt thereof.

In some embodiments, the compound of Formula (I) is of the formula:

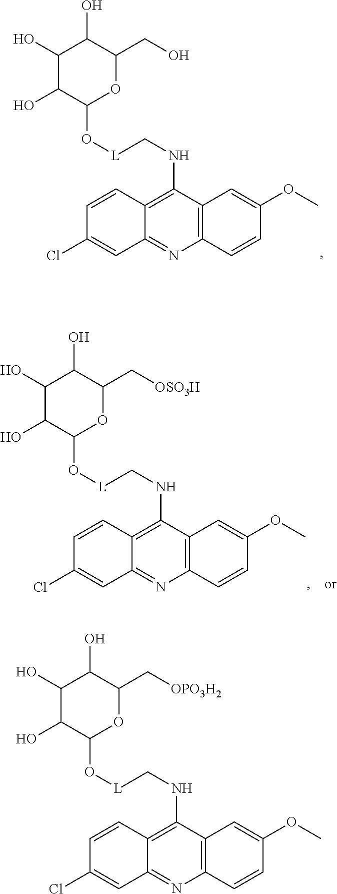

or a pharmaceutically acceptable salt thereof.

In some embodiments, the compound of Formula (I) is of the formula:

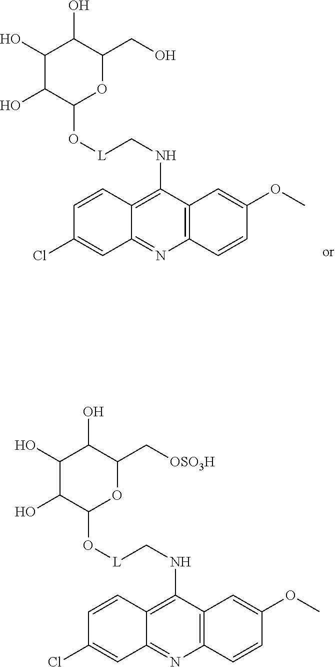

or a pharmaceutically acceptable salt thereof.

In some embodiments, the compound of Formula (I) is of the formula:

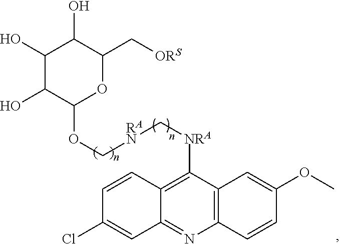

or a pharmaceutically acceptable salt thereof, wherein each instance of n is independently an integer from 1-8, inclusive. In some embodiments, each instance of R^{A} is independently selected from hydrogen and substituted or unsubstituted C₁₋₆ alkyl. In some embodiments, one instance of R^{A} is hydrogen and one instance of R^{A} is ethyl. In some embodiments, R^{S} is —H, —SO₃H, or —PO₃H₂. In some embodiments, R^{S} is —H or —SO₃H.

In some embodiments, the compound of Formula (I) is of the formula:

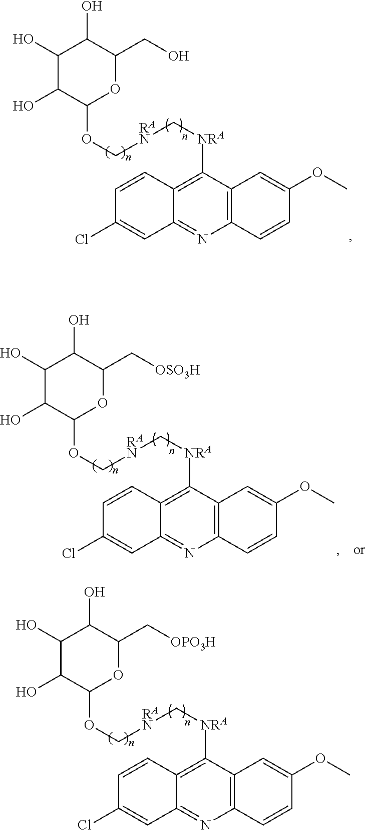

or a pharmaceutically acceptable salt thereof, wherein each instance of n is independently an integer from 1-8, inclusive. In some embodiments, each instance of R^{A} is independently selected from hydrogen and substituted or unsubstituted C₁₋₆ alkyl. In some embodiments, one instance of R^{A} is hydrogen and one instance of R^{A} is ethyl.

In some embodiments, the compound of Formula (I) is of the formula:

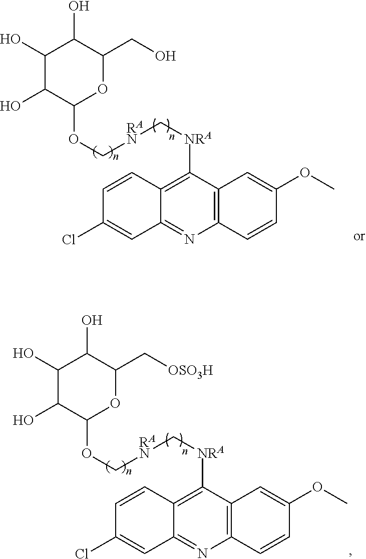

or a pharmaceutically acceptable salt thereof, wherein each instance of n is independently an integer from 1-8, inclusive. In some embodiments, each instance of R^{A} is independently selected from hydrogen and substituted or unsubstituted C₁₋₆ alkyl. In some embodiments, one instance of R^{A} is hydrogen and one instance of R^{A} is ethyl.

In some embodiments, the compound of Formula (I) is of the formula:

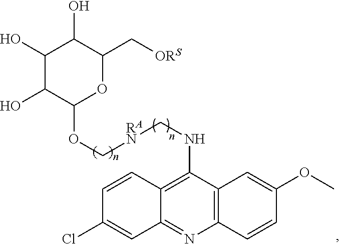

or a pharmaceutically acceptable salt thereof, wherein each instance of n is independently an integer from 1-8, inclusive. In some embodiments, R^{A} is selected from hydrogen and substituted or unsubstituted C₁₋₆ alkyl. In some embodiments, R^{A} hydrogen or ethyl. In some embodiments, R^{S} is —H, —SO₃H, or —PO₃H₂. In some embodiments, R^{S} is —H or —SO₃H.

In some embodiments, the compound of Formula (I) is of the formula:

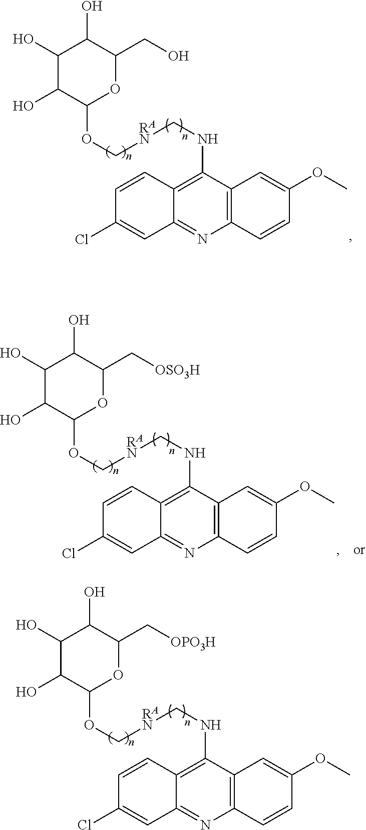

or a pharmaceutically acceptable salt thereof, wherein each instance of n is independently an integer from 1-8, inclusive. In some embodiments, R^{A} is selected from hydrogen and substituted or unsubstituted C₁₋₆ alkyl. In some embodiments, R^{A} hydrogen or ethyl.

In some embodiments, the compound of Formula (I) is of the formula:

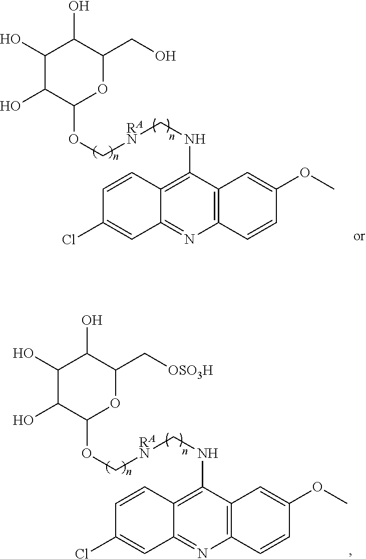

or a pharmaceutically acceptable salt thereof, wherein each instance of n is independently an integer from 1-8, inclusive. In some embodiments, R^{A} is selected from hydrogen and substituted or unsubstituted C₁₋₆ alkyl. In some embodiments, R^{A} hydrogen or ethyl.

In some embodiments, the compound of Formula (I) is of the formula:

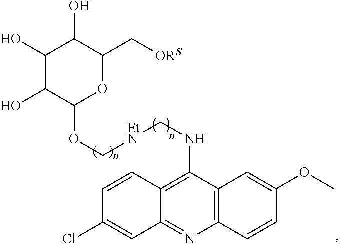

or a pharmaceutically acceptable salt thereof, wherein each instance of n is independently an integer from 1-8, inclusive. In some embodiments, R^{S} is —H, —SO₃H, or —PO₃H₂. In some embodiments, R^{S} is —H or —SO₃H.

In some embodiments, the compound of Formula (I) is of the formula:

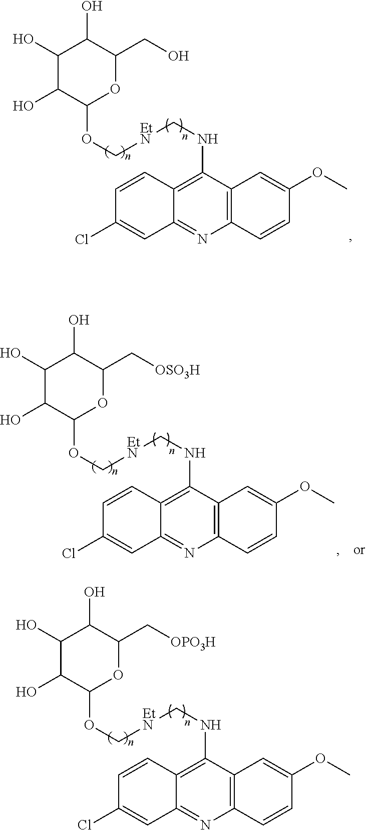

or a pharmaceutically acceptable salt thereof, wherein each instance of n is independently an integer from 1-8, inclusive.

In some embodiments, the compound of Formula (I) is of the formula:

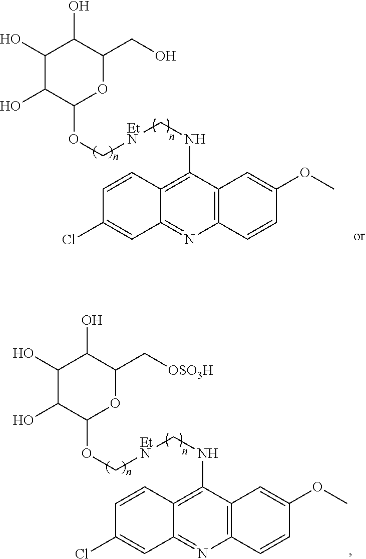

or a pharmaceutically acceptable salt thereof, wherein each instance of n is independently an integer from 1-8, inclusive.

In some embodiments, the compound of Formula (I) is of the formula:

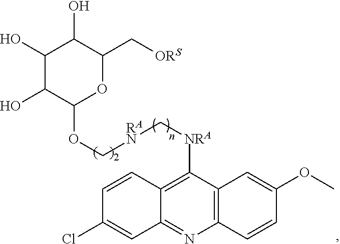

or a pharmaceutically acceptable salt thereof. In some embodiments, each instance of R^{A} is independently selected from hydrogen and substituted or unsubstituted C₁₋₆ alkyl. In some embodiments, R^{S} is —H, —SO₃H, or —PO₃H₂. In some embodiments, R^{S} is —H or —SO₃H.

In some embodiments, the compound of Formula (I) is of the formula:

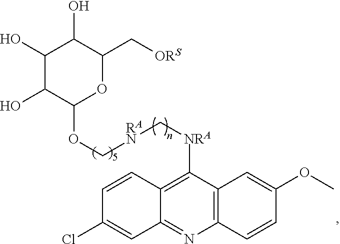

or a pharmaceutically acceptable salt thereof. In some embodiments, each instance of R^{A} is independently selected from hydrogen and substituted or unsubstituted C₁₋₆ alkyl. In some embodiments, R^{S} is —H, —SO₃H, or —PO₃H₂. In some embodiments, R^{S} is —H or —SO₃H.

In some embodiments, the compound of Formula (I) is of the formula:

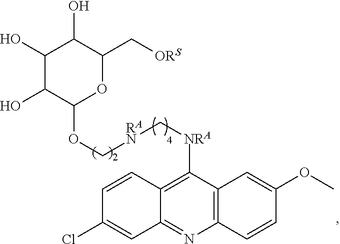

or a pharmaceutically acceptable salt thereof. In some embodiments, each instance of R^{A} is independently selected from hydrogen and substituted or unsubstituted C₁₋₆ alkyl. In some embodiments, R^{S} is —H, —SO₃H, or —PO₃H₂. In some embodiments, R^{S} is —H or —SO₃H.

In some embodiments, the compound of Formula (I) is of the formula:

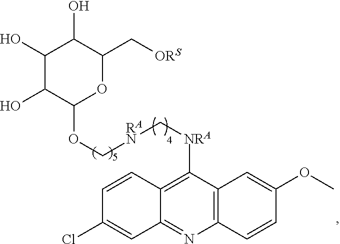

or a pharmaceutically acceptable salt thereof. In some embodiments, each instance of R^{A} is independently selected from hydrogen and substituted or unsubstituted C₁₋₆ alkyl. In some embodiments, R^{S} is —H, —SO₃H, or —PO₃H₂. In some embodiments, R^{S} is —H or —SO₃H.

In some embodiments, the compound of Formula (I) is of the formula:

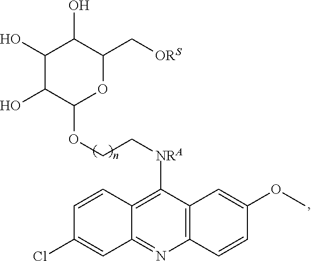

or a pharmaceutically acceptable salt thereof, wherein n is an integer from 1-8, inclusive. In some embodiments, R^{S} is —H, —SO₃H, or —P03H2. In some embodiments, R^{S} is —H or —SO₃H.

In some embodiments, the compound of Formula (I) is of the formula:

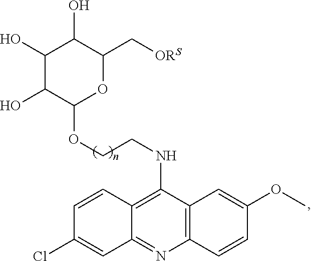

or a pharmaceutically acceptable salt thereof, wherein n is an integer from 1-8, inclusive.

In some embodiments, the compound of Formula (I) is of the formula:

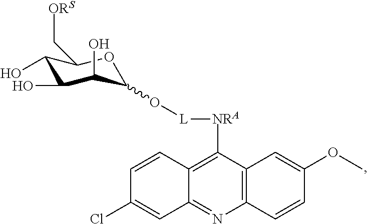

or a pharmaceutically acceptable salt thereof. In some embodiments, R^{S} is —H, —SO₃H, or —PO₃H₂. In some embodiments, R^{S} is —H or —SO₃H.

In some embodiments, the compound of Formula (I) is of the formula:

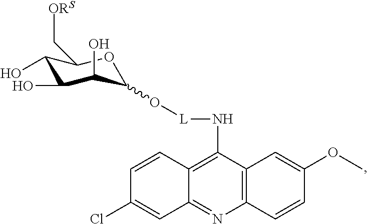

or a pharmaceutically acceptable salt thereof. In some embodiments, R^{S} is —H, —SO₃H, or —PO₃H₂. In some embodiments, R^{S} is —H or —SO₃H.

In some embodiments, the compound of Formula (I) is of the formula:

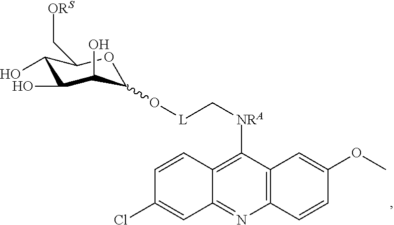

or a pharmaceutically acceptable salt thereof. In some embodiments, R^{S} is —H, —SO₃H, or —PO₃H₂. In some embodiments, R^{S} is —H or —SO₃H.

In some embodiments, the compound of Formula (I) is of the formula:

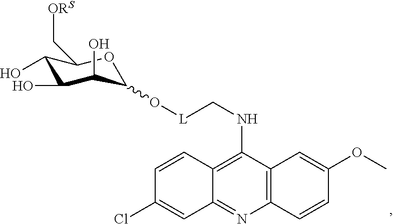

or a pharmaceutically acceptable salt thereof. In some embodiments, R^{S} is —H, —SO₃H, or —PO₃H₂. In some embodiments, R^{S} is —H or —SO₃H.

In some embodiments, the compound of Formula (I) is of the formula:

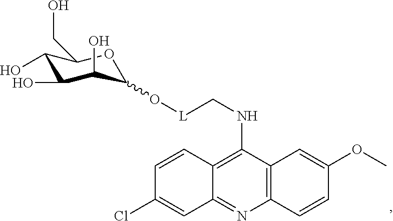

or a pharmaceutically acceptable salt thereof.

In some embodiments, the compound of Formula (I) is of the formula:

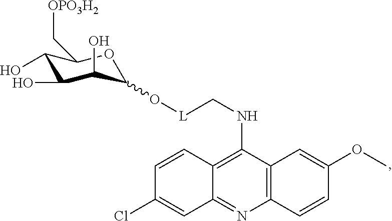

or a pharmaceutically acceptable salt thereof.

In some embodiments, the compound of Formula (I) is of the formula:

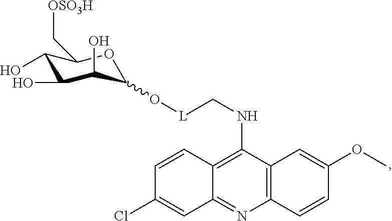

or a pharmaceutically acceptable salt thereof.

In some embodiments, the compound of Formula (I) is of the formula:

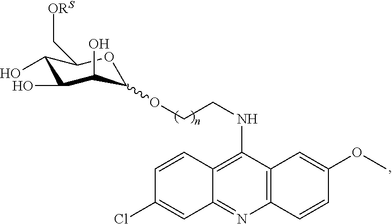

or a pharmaceutically acceptable salt thereof, wherein n is an integer from 1-8, inclusive.

In some embodiments, the compound of Formula (I) is of the formula:

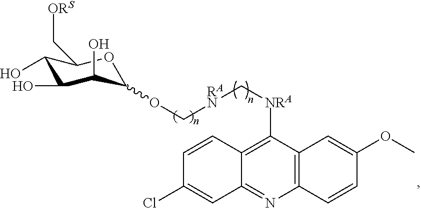

or a pharmaceutically acceptable salt thereof, wherein each instance of n is independently an integer from 1-8, inclusive. In some embodiments, R^{S} is —H, —SO₃H, or —PO₃H₂. In some embodiments, R^{S} is —H or —SO₃H.

In some embodiments, the compound of Formula (I) is of the formula:

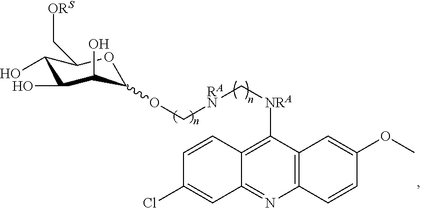

or a pharmaceutically acceptable salt thereof, wherein each instance of n is independently an integer from 1-8, inclusive. In some embodiments, R^{S} is —H, —SO₃H, or —PO₃H₂. In some embodiments, R^{S} is —H or —SO₃H. In some embodiments, R^{A} is hydrogen, methyl, or ethyl. In some embodiments, R^{A} is ethyl.

In some embodiments, the ratio of sugar to the DNA-interacting agent is 2:1. In some embodiments, the ratio of sugar to the DNA-interacting agent is 1:1. In some embodiments, the ratio of sugar to DNA intercalating agent is 2:1. In some embodiments, the ratio of sugar to DNA intercalating agent is 1:1.

In certain embodiments, a compound of Formula (I) is of the formula:

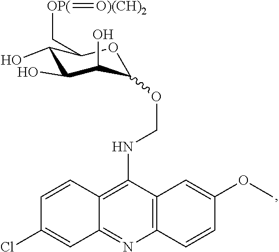

or salt thereof.

In certain embodiments, a compound of Formula (I) of the formula:

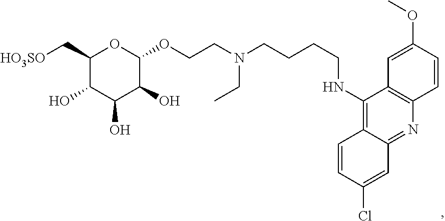

or salt thereof.

In certain embodiments, a compound of Formula (I) is of the formula:

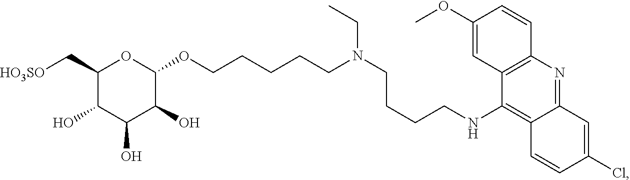

or salt thereof.

In certain embodiments, a compound of Formula (I) is of the formula:

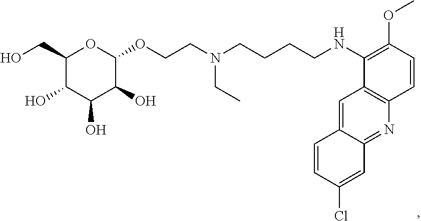 [SMILES: CCN(CCCCNc1c(OC)ccc2nc3cc(Cl)ccc3cc12)CCO[C@H]1O[C@H](CO)[C@@H](O)[C@H](O)[C@@H]1O]

or salt thereof.

In certain embodiments, a compound of Formula (I) is of the formula:

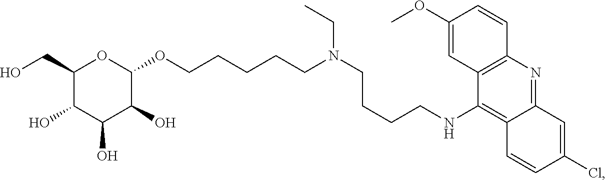 [SMILES: CCN(CCCCCO[C@H]1O[C@H](CO)[C@@H](O)[C@H](O)[C@@H]1O)CCCCNc1c2ccc(Cl)cc2nc2ccc(OC)cc12]

or salt thereof.

### DNA-Interacting Agents (R²)

The compounds of Formula (I), and subgenera thereof, comprise R², wherein R² is a DNA-interacting agent.

In certain embodiments, the DNA-interacting agent is a DNA intercalating agent. In some embodiments, R² is a DNA intercalating agent. In some embodiments, the DNA intercalating agent is selected from the group consisting of an acridine (e.g., 6-chloro-2-methoxyacridine, 6-chloro-2-methoxyacridin-9-amine), actinomycins, acriflavine, amsacrine, amonafide, anthracyclines, berberine, benzophenone, daunomycin, dihydroethidium, dimidium, doxorubicin, ethidium, lucanthone, hycanthone, naphthalenetetracarboxylic diimides, nogalomycin, oxazolopyridocarbozole, phenanthrene, 1,10-phenathroline, phenanthridine, proflavine, propidium, pyrazoloacridine, quinacrine, thalidomide, thioxanthone, tilorone, and other organic molecules comprising a planar aromatic or heteroaromatic moiety capable of stacking between the nucleobases of DNA, or derivatives thereof. In some embodiments, the DNA intercalating agent is selected from the group consisting of acridine (e.g., 6-chloro-2-methoxyacridine, 6-chloro-2-methoxyacridin-9-amine), berberine, daunomycin, dihydroethidium, dimidium, doxorubicin, ethidium, lucanthone, hycanthone, phenanthrene, phenanthridine, proflavine, propidium, quinacrine, thalidomide, thioxanthone, and tilorone. In some embodiments, R² is amsacrine, propidium, 1,10-phenanthroline, amonafide, pyrazoloacridine, or acriflavine. In some embodiments, R² is substituted or unsubstituted quinacrine. In some embodiments, R² is substituted or unsubstituted acridine. In some embodiments, R² is 6-chloro-2-methoxyacridine. In some embodiments, R² is 6-chloro-2-methoxyacridin-9-amine. In some embodiments, R² is substituted or unsubstituted actinomycin. In some embodiments, R² is substituted or unsubstituted acriflavine. In some embodiments, R² is substituted or unsubstituted amsacrine. In some embodiments, R² is substituted or unsubstituted amonafide. In some embodiments, R² is a substituted or unsubstituted anthracycline. In some embodiments, R² is substituted or unsubstituted berberine. In some embodiments, R² is a substituted or unsubstituted benzophenone. In some embodiments, R² is substituted or unsubstituted daunomycin. In some embodiments, R² is substituted or unsubstituted dihydroethidium. In some embodiments, R² is substituted or unsubstituted dimidium. In some embodiments, R² is substituted or unsubstituted doxorubicin. In some embodiments, R² is substituted or unsubstituted ethidium. In some embodiments, R² is substituted or unsubstituted lucanthone. In some embodiments, R² is substituted or unsubstituted hycanthone. In some embodiments, R² is substituted or unsubstituted naphthalenetetracarboxylic diimide. In some embodiments, R² is substituted or unsubstituted nogalomycin. In some embodiments, R² is substituted or unsubstituted oxazolopyridocarbozole. In some embodiments, R² is substituted or unsubstituted phenanthrene. In some embodiments, R² is substituted or unsubstituted 1,10-phenathroline. In some embodiments, R² is substituted or unsubstituted phenanthridine. In some embodiments, R² is substituted or unsubstituted proflavine. In some embodiments, R² is substituted or unsubstituted propidium. In some embodiments, R² is substituted or unsubstituted pyrazoloacridine. In some embodiments, R² is substituted or unsubstituted thalidomide. In some embodiments, R² is substituted or unsubstituted thioxanthone. In some embodiments, R² is substituted or unsubstituted tilorone. In some embodiments, R² is a substituted or unsubstituted organic molecule comprising a planar aromatic or heteroaromatic moiety capable of stacking between the nucleobases of DNA. In some embodiments, R² is unsubstituted quinacrine. In some embodiments, R² is unsubstituted acridine. In some embodiments, R² is 6-chloro-2-methoxyacridine. In some embodiments, R² is 6-chloro-2-methoxyacridin-9-amine. In some embodiments, R² is unsubstituted actinomycin. In some embodiments, R² is unsubstituted acriflavine. In some embodiments, R² is unsubstituted amsacrine. In some embodiments, R² is unsubstituted amonafide. In some embodiments, R² is a unsubstituted anthracycline. In some embodiments, R² is unsubstituted berberine. In some embodiments, R² is a unsubstituted benzophenone. In some embodiments, R² is unsubstituted daunomycin. In some embodiments, R² is unsubstituted dihydroethidium. In some embodiments, R² is unsubstituted dimidium. In some embodiments, R² is unsubstituted doxorubicin. In some embodiments, R² is unsubstituted ethidium. In some embodiments, R² is unsubstituted lucanthone. In some embodiments, R² is unsubstituted hycanthone. In some embodiments, R² is unsubstituted naphthalenetetracarboxylic diimide. In some embodiments, R² is unsubstituted nogalomycin. In some embodiments, R² is unsubstituted oxazolopyridocarbozole. In some embodiments, R² is unsubstituted phenanthrene. In some embodiments, R² is unsubstituted 1,10-phenathroline. In some embodiments, R² is unsubstituted phenanthridine. In some embodiments, R² is unsubstituted proflavine. In some embodiments, R² is unsubstituted propidium. In some embodiments, R² is unsubstituted pyrazoloacridine. In some embodiments, R² is unsubstituted thalidomide. In some embodiments, R² is unsubstituted thioxanthone. In some embodiments, R² is unsubstituted tilorone. In some embodiments, R² is a unsubstituted organic molecule comprising a planar aromatic or heteroaromatic moiety capable of stacking between the nucleobases of DNA. In some embodiments, R² is substituted quinacrine. In some embodiments, R² is substituted acridine. In some embodiments, R² is 6-chloro-2-methoxyacridine. In some embodiments, R² is 6-chloro-2-methoxyacridin-9-amine. In some embodiments, R² is substituted actinomycin In some embodiments, R² is substituted acriflavine. In some embodiments, R² is substituted amsacrine. In some embodiments, R² is substituted amonafide. In some embodiments, R² is a substituted anthracycline. In some embodiments, R² is substituted berberine. In some embodiments, R² is a substituted benzophenone. In some embodiments, R² is substituted daunomycin. In some embodiments, R² is substituted dihydroethidium. In some embodiments, R² is substituted dimidium. In some embodiments, R² is substituted doxorubicin. In some embodiments, R² is substituted ethidium. In some embodiments, R² is substituted lucanthone. In some embodiments, R² is substituted hycanthone. In some embodiments, R² is substituted naphthalenetetracarboxylic diimide. In some embodiments, R² is substituted nogalomycin. In some embodiments, R² is substituted oxazolopyridocarbozole. In some embodiments, R² is substituted phenanthrene. In some embodiments, R² is substituted 1,10-phenathroline. In some embodiments, R² is substituted phenanthridine. In some embodiments, R² is substituted proflavine. In some embodiments, R² is substituted propidium. In some embodiments, R² is substituted pyrazoloacridine. In some embodiments, R² is substituted thalidomide. In some embodiments, R² is substituted thioxanthone. In some embodiments, R² is substituted tilorone. In some embodiments, R² is a substituted organic molecule comprising a planar aromatic or heteroaromatic moiety capable of stacking between the nucleic acid bases.

In some embodiments, R² is an organic molecule, a molecule-oligonucleotide conjugate, or an organometallic compound. In certain embodiments, R² is a molecule that non-covalently interacts with DNA, a DNA groove-binding molecule, or intercalator-groove binding hybrid molecule. In some embodiments, the organic molecule or molecule that non-covalently interacts with DNA is ethidium bromide, quinacrine, an anthracycline, an actinomycin, daunomycin, or nogalomycin. In some embodiments, the DNA groove-binding molecule is a major groove binder or a minor groove binder. In certain embodiments, the DNA-groove-binding molecule is a triplex-forming oligonucleotide, a peptide nucleic acid, netropsin, SAPI, distamycin, Hoechst 33258, bis-benzimidazole derivatives, berenil, a polyamide composed of N-methylimidazole, N-methyl pyrrole, and N-methyl-3-hydroxy-pyrrole, a N-methylpyrrole oligopeptide, a N-methylimidazole based compound, β-alanine-linked polyamides, a hairpin polyamide, symmetric dimeric bis-benzimidazole, or a tripyrrole peptide-Hoechst conjugate. In certain embodiments, the major groove binding molecule is a triplex-forming oligonucleotide or a peptide nucleic acid. In some embodiments, the minor groove binding molecule is netropsin, SAPI, distamycin, Hoechst 33258, bis-benzimidazole derivatives, berenil, a polyamide composed of N-methylimidazole, N-methyl pyrrole, and N-methyl-3-hydroxy-pyrrole, a N-methylpyrrole oligopeptide, a N-methylimidazole based compound, β-alanine-linked polyamides, a hairpin polyamide, symmetric dimeric bis-benzimidazole, or a tripyrrole peptide-Hoechst conjugate. In some embodiments, the intercalator-groove binding hybrid is a hairpin polyamide, NetAmsa, or an analog of amsacrine, ellipticine, anthraquinones, or mitoxantrone. In some embodiments, small molecule oligonucleotide conjugate is a deoxy-oligonucleotide conjugated to an intercalator. In some embodiments, the small molecule oligonucleotide conjugate is Hoechst-oligonucleotide conjugate, distamycin-oligonucleotide conjugate, or CDPI₃. In some embodiments, R² is a deoxy-oligonucleotide conjugated to an intercalator. In some embodiments, R² is a hairpin polyamide. In some embodiments, R² is a major groove binder. In some embodiments, R² is a minor groove binder. In some embodiments, R² is a molecule-oligonucleotide conjugate. In some embodiments, R² is a N-methylimidazole based compound. In some embodiments, R² is a N-methylpyrrole oligopeptide. In some embodiments, R² is a peptide nucleic acid. In some embodiments, R² is a polyamide composed of N-methyl pyrrole. In some embodiments, R² is a polyamide composed of N-methyl-3-hydroxy-pyrrole. In some embodiments, R² is a polyamide composed of N-methylimidazole. In some embodiments, R² is a triplex-forming oligonucleotide. In some embodiments, R² is a tripyrrole peptide-Hoechst conjugate. In some embodiments, R² is an actinomycin. In some embodiments, R² is an analog of amsacrine. In some embodiments, R² is an analog of anthraquinones. In some embodiments, R² is an analog of ellipticine. In some embodiments, R² is an analog of mitoxantrone. In some embodiments, R² is an anthracycline. In some embodiments, R² is an organometallic compound. In some embodiments, R² is berenil. In some embodiments, R² is bis-benzimidazole derivatives. In some embodiments, R² is CDPI₃. In some embodiments, R² is daunomycin. In some embodiments, R² is distamycin. In some embodiments, R² is a distamycin-oligonucleotide conjugate. In some embodiments, R² is a DNA groove-binding molecule. In some embodiments, R² is ethidium bromide. In some embodiments, R² is Hoechst 33258. In some embodiments, R² is Hoechst-oligonucleotide conjugate. In some embodiments, R² is an intercalator-groove binding hybrid molecule. In some embodiments, R² is a molecule that non-covalently interacts with DNA. In some embodiments, R² is NetAmsa. In some embodiments, R² is netropsin. In some embodiments, R² is nogalomycin. In some embodiments, R² is an organic molecule. In some embodiments, R² is quinacrine. In some embodiments, R² is SAPI. In some embodiments, R² is symmetric dimeric bis-benzimidazole. In some embodiments, R² is β-alanine-linked polyamides. In some embodiments, R² is substituted or unsubstituted ethidium bromide. In some embodiments, R² is substituted or unsubstituted quinacrine. In some embodiments, R² is an substituted or unsubstituted anthracycline. In some embodiments, R² is an substituted or unsubstituted actinomycin. In some embodiments, R² is substituted or unsubstituted daunomycin. In some embodiments, R² is substituted or unsubstituted nogalomycin. In some embodiments, R² is a substituted or unsubstituted triplex-forming oligonucleotide. In some embodiments, R² is a substituted or unsubstituted peptide nucleic acid. In some embodiments, R² is substituted or unsubstituted netropsin. In some embodiments, R² is substituted or unsubstituted SAPI. In some embodiments, R² is substituted or unsubstituted distamycin. In some embodiments, R² is substituted or unsubstituted Hoechst 33258. In some embodiments, R² is a substituted or unsubstituted bis-benzimidazole derivative. In some embodiments, R² is substituted or unsubstituted berenil. In some embodiments, R² is a substituted or unsubstituted polyamide composed of N-methylimidazole, N-methyl pyrrole, and N-methyl-3-hydroxy-pyrrole. In some embodiments, R² is a substituted or unsubstituted polyamide composed of N-methylimidazole. In some embodiments, R² is a substituted or unsubstituted polyamide composed of N-methyl pyrrole. In some embodiments, R² is a substituted or unsubstituted polyamide composed of N-methyl-3-hydroxy-pyrrole. In some embodiments, R² is a substituted or unsubstituted N-methylpyrrole oligopeptide. In some embodiments, R² is a substituted or unsubstituted N-methylimidazole based compound. In some embodiments, R² is a substituted or unsubstituted β-alanine-linked polyamide. In some embodiments, R² is a substituted or unsubstituted hairpin polyamide. In some embodiments, R² is substituted or unsubstituted symmetric dimeric bis-benzimidazole. In some embodiments, R² is a substituted or unsubstituted tripyrrole peptide-Hoechst conjugate. In some embodiments, R² is substituted or unsubstituted NetAmsa. In some embodiments, R² is a substituted or unsubstituted analog of amsacrine. In some embodiments, R² is a substituted or unsubstituted analog of ellipticine. In some embodiments, R² is a substituted or unsubstituted analog of anthraquinones. In some embodiments, R² is a substituted or unsubstituted analog of mitoxantrone. In some embodiments, R² is substituted or unsubstituted Hoechst-oligonucleotide conjugate. In some embodiments, R² is a substituted or unsubstituted distamycin-oligonucleotide conjugate. In some embodiments, R² is substituted or unsubstituted CDPI₃. In some embodiments, R² is substituted ethidium bromide. In some embodiments, R² is substituted quinacrine. In some embodiments, R² is an substituted anthracycline. In some embodiments, R² is an substituted actinomycin. In some embodiments, R² is substituted daunomycin. In some embodiments, R² is substituted nogalomycin. In some embodiments, R² is a substituted triplex-forming oligonucleotide. In some embodiments, R² is a substituted peptide nucleic acid. In some embodiments, R² is substituted netropsin. In some embodiments, R² is substituted SAPI. In some embodiments, R² is substituted distamycin. In some embodiments, R² is substituted Hoechst 33258. In some embodiments, R² is a substituted bis-benzimidazole derivative. In some embodiments, R² is substituted berenil. In some embodiments, R² is a substituted polyamide composed of N-methylimidazole, N-methyl pyrrole, and N-methyl-3-hydroxy-pyrrole. In some embodiments, R² is a substituted polyamide composed of N-methylimidazole. In some embodiments, R² is a substituted polyamide composed of N-methyl pyrrole. In some embodiments, R² is a substituted polyamide composed of N-methyl-3-hydroxy-pyrrole. In some embodiments, R² is a substituted N-methylpyrrole oligopeptide. In some embodiments, R² is a substituted N-methylimidazole based compound. In some embodiments, R² is a substituted β-alanine-linked polyamide. In some embodiments, R² is a substituted hairpin polyamide. In some embodiments, R² is substituted symmetric dimeric bis-benzimidazole. In some embodiments, R² is a substituted tripyrrole peptide-Hoechst conjugate. In some embodiments, R² is substituted NetAmsa. In some embodiments, R² is a substituted analog of amsacrine. In some embodiments, R² is a substituted analog of ellipticine. In some embodiments, R² is a substituted analog of anthraquinones. In some embodiments, R² is a substituted analog of mitoxantrone. In some embodiments, R² is substituted Hoechst-oligonucleotide conjugate. In some embodiments, R² is a substituted distamycin-oligonucleotide conjugate. In some embodiments, R² is substituted CDPI₃. In some embodiments, R² is unsubstituted ethidium bromide. In some embodiments, R² is unsubstituted quinacrine. In some embodiments, R² is an unsubstituted anthracycline. In some embodiments, R² is an unsubstituted actinomycin. In some embodiments, R² is unsubstituted daunomycin. In some embodiments, R² is unsubstituted nogalomycin. In some embodiments, R² is a unsubstituted triplex-forming oligonucleotide. In some embodiments, R² is a unsubstituted peptide nucleic acid. In some embodiments, R² is unsubstituted netropsin. In some embodiments, R² is unsubstituted SAPI. In some embodiments, R² is unsubstituted distamycin. In some embodiments, R² is unsubstituted Hoechst 33258. In some embodiments, R² is a unsubstituted bis-benzimidazole derivative. In some embodiments, R² is unsubstituted berenil. In some embodiments, R² is a unsubstituted polyamide composed of N-methylimidazole, N-methyl pyrrole, and N-methyl-3-hydroxy-pyrrole. In some embodiments, R² is a unsubstituted polyamide composed of N-methylimidazole. In some embodiments, R² is a unsubstituted polyamide composed of N-methyl pyrrole. In some embodiments, R² is a unsubstituted polyamide composed of N-methyl-3-hydroxy-pyrrole. In some embodiments, R² is a unsubstituted N-methylpyrrole oligopeptide. In some embodiments, R² is a unsubstituted N-methylimidazole based compound. In some embodiments, R² is a unsubstituted β-alanine-linked polyamide. In some embodiments, R² is a unsubstituted hairpin polyamide. In some embodiments, R² is unsubstituted symmetric dimeric bis-benzimidazole. In some embodiments, R² is a unsubstituted tripyrrole peptide-Hoechst conjugate. In some embodiments, R² is unsubstituted NetAmsa. In some embodiments, R² is a unsubstituted analog of amsacrine. In some embodiments, R² is a unsubstituted analog of ellipticine. In some embodiments, R² is a unsubstituted analog of anthraquinones. In some embodiments, R² is a unsubstituted analog of mitoxantrone. In some embodiments, R² is unsubstituted Hoechst-oligonucleotide conjugate. In some embodiments, R² is a unsubstituted distamycin-oligonucleotide conjugate. In some embodiments, R² is unsubstituted CDPI₃.

In some embodiments, R² is

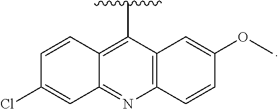 [SMILES: *c1c2ccc(Cl)cc2nc2ccc(OC)cc12]

In some embodiments, R² is

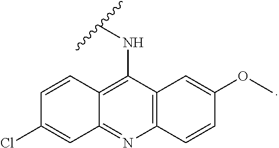 [SMILES: *Nc1c2ccc(Cl)cc2nc2ccc(OC)cc12]

In some embodiments, R² is

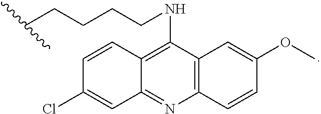 [SMILES: *CCCCNc1c2ccc(Cl)cc2nc2ccc(OC)cc12]

In some embodiments, R² is

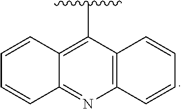 [SMILES: *c1c2ccccc2nc2ccccc12]

In some embodiments, R² is

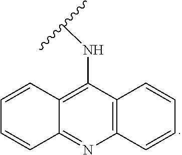 [SMILES: *Nc1c2ccccc2nc2ccccc12]

In some embodiments, R² is

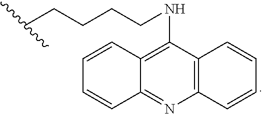 [SMILES: *CCCCNc1c2ccccc2nc2ccccc12]

In some embodiments, R² is optionally substituted

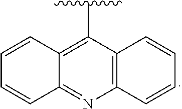 [SMILES: *c1c2ccccc2nc2ccccc12]

In some embodiments, R² is optionally substituted

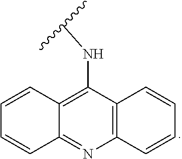 [SMILES: *Nc1c2ccccc2nc2ccccc12]

In some embodiments, R² is optionally substituted

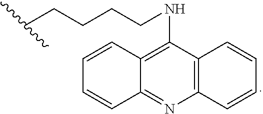 [SMILES: *CCCCNc1c2ccccc2nc2ccccc12]

In some embodiments, R² is non-toxic. In some embodiments, the DNA intercalating agent is non-toxic.

In certain embodiments, R² is non-carcinogenic. In certain embodiments, the DNA intercalating agent is non-carcinogenic.

In some embodiments, R² does not induce DNA damage. In some embodiments, the DNA-interacting agent does not induce DNA damage. In some embodiments, the DNA intercalating agent does not induce DNA damage.

In some embodiments, R² is FDA approved. In some embodiments, the DNA interacting agent is FDA approved. In some embodiments, the DNA intercalating agent is FDA approved,

### Linker

The compounds of Formula (I), and subgenera thereof, comprise L, a linker, wherein L is substituted C₁₋₂₀ alkylene or unsubstituted C₁₋₂₀ alkylene, wherein: optionally one or more backbone carbon atoms in the substituted C₁₋₂₀ alkylene or unsubstituted C₁₋₂₀ alkylene are independently replaced with —NR^{A}—, —O—, —C(═O)—, —S—, substituted or unsubstituted carbocyclylene, substituted or unsubstituted heterocyclylene, substituted or unsubstituted arylene, or substituted or unsubstituted heteroarylene.

In some embodiments, L is substituted C₁₋₂₀ alkylene or unsubstituted C₁₋₂₀ alkylene, wherein: optionally one or more backbone carbon atoms in the substituted C₁₋₂₀ alkylene or unsubstituted C₁₋₂₀ alkylene are independently replaced with —NR^{A}—, —O—, —C(═O)—, —S—, substituted or unsubstituted carbocyclylene, substituted or unsubstituted arylene, or substituted or unsubstituted heteroarylene. In certain embodiments, the one or more backbone carbon atoms in the substituted C₁₋₂₀ alkylene or unsubstituted C₁₋₂₀ alkylene are independently replaced with —NR^{A}—, —O—, or —C(═O)—. In some embodiments, the one or more backbone carbon atoms in the substituted C₁₋₁₀ alkylene or unsubstituted C₁₋₁₀ alkylene are independently replaced with —NR^{A}, —O—, or —C(═O)—.

In some embodiments, L is substituted C₁₋₂₀ alkylene or substituted C₁₋₁₀alkylene wherein the alkylene is substituted with one or more substituted or unsubstituted sugars (independent of R¹). In some embodiments, L is C₁₋₂₀ alkylene substituted with one or more substituted or unsubstituted sugars. In some embodiments, L is C₁₋₁₀ alkylene substituted with one or more substituted or unsubstituted sugars. In some embodiments, L is C₁₋₂₀ alkylene substituted with one substituted or unsubstituted sugar. In some embodiments, L is C₁₋₁₀ alkylene substituted with one substituted or unsubstituted sugar. In some embodiments, L is C₁₋₂₀ alkylene substituted with two substituted or unsubstituted sugars. In some embodiments, L is C₁₋₁₀ alkylene substituted two one substituted or unsubstituted sugars. In some embodiments, L is C₁₋₂₀ alkylene substituted with one or more substituted sugars. In some embodiments, L is C₁₋₁₀ alkylene substituted with one or more substituted sugars. In some embodiments, L is C₁₋₂₀ alkylene substituted with one substituted sugar. In some embodiments, L is C₁₋₁₀ alkylene substituted with one substituted sugar. In some embodiments, L is C₁₋₂₀ alkylene substituted with two substituted sugars. In some embodiments, L is C₁₋₁₀ alkylene substituted two one substituted sugars. In some embodiments, L is C₁₋₂₀ alkylene substituted with one or more unsubstituted sugars. In some embodiments, L is C₁₋₁₀ alkylene substituted with one or more unsubstituted sugars. In some embodiments, L is C₁₋₂₀ alkylene substituted with one unsubstituted sugar. In some embodiments, L is C₁₋₁₀ alkylene substituted with one unsubstituted sugar. In some embodiments, L is C₁₋₂₀ alkylene substituted with two unsubstituted sugars. In some embodiments, L is C₁₋₁₀ alkylene substituted two one unsubstituted sugars.

In some embodiments, L is unsubstituted C₁₋₁₀ alkylene. In some embodiments, L is unsubstituted C₁ alkylene. In some embodiments, L is unsubstituted C₂ alkylene. In some embodiments, L is unsubstituted C₃ alkylene. In some embodiments, L is unsubstituted C₄ alkylene. In some embodiments, L is unsubstituted C₅ alkylene. In some embodiments, L is unsubstituted C₆ alkylene. In some embodiments, L is unsubstituted C₇ alkylene. In some embodiments, L is unsubstituted C₈ alkylene. In some embodiments, L is unsubstituted C₉ alkylene. In some embodiments, L is unsubstituted C₁₀ alkylene.

In some embodiments, L is substituted C₁₋₁₀ alkylene. In some embodiments, L is substituted C₁ alkylene. In some embodiments, L is substituted C₂ alkylene. In some embodiments, L is substituted C₃ alkylene. In some embodiments, L is substituted C₄ alkylene. In some embodiments, L is substituted C₅ alkylene. In some embodiments, L is substituted C₆ alkylene. In some embodiments, L is substituted C₇ alkylene. In some embodiments, L is substituted C₈ alkylene. In some embodiments, L is substituted C₉ alkylene. In some embodiments, L is substituted C₁₀ alkylene.

As provided herein, the linker, L, may comprise n, which is an integer from 1-8 inclusive. In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8.

In some embodiments, the linker, L, is a C₁₋₁₀alkylene,

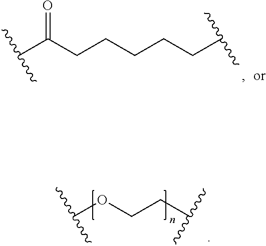

In some embodiments, the linker, L, is attached to the DNA-interacting agent (R²) via a bond, amide (i.e., —NHC(═O)— or —C(═O)NH—), ester (i.e., —OC(═O)— or —C(═O)O—), amine (i.e., —NH—), or ether (—O—) moiety. In some embodiments, the linker, L, is attached to the DNA-interacting agent (R²) via a single bond.

In some embodiments, the linker, L, is attached to the sugar (R¹) via a bond, amide (i.e., —NHC(═O)— or —C(═O)NH—), ester (i.e., —OC(═O)— or —C(═O)O—), amine (i.e., —NH—), or ether (—O—) moiety. In some embodiments, the linker, L, is attached to the sugar (R¹) via a single bond.

In some embodiments, the linker, L, is

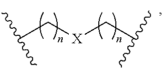

wherein each instance of n is independently an integer from 1-8, inclusive, and X is —O—, —NR^{A}, —S—, —C(═O)—, —NR^{A}C(═O)—, —C(═O)NR^{A}—, —OC(═O)—, or —C(═O)O—. In some embodiments, the linker, L, is

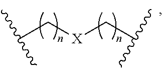

wherein each instance of n is independently selected from 2, 4, and 5, and X is —O—, —NR^{A}, —S—, —C(═O)—, —NR^{A}C(═O)—, —C(═O)NR^{A}—, —OC(═O)—, or —C(═O)O—. In some embodiments, the linker, L, is

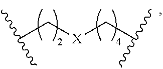

wherein X is —O—, NR^{A}, —S—, —C(═O)—, —NR^{A}C(═O)—, —C(═O)NR^{A}—, —OC(═O)—, or —C(═O)O—. In some embodiments, the linker, L, is

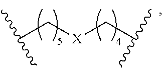

wherein X is —O—, —NR^{A}—, —S—, —C(═O)—, —NR^{A}C(═O)—, —C(═O)NR^{A}—, —OC(═O)—, or —C(═O)O—.

In some embodiments, the linker, L, is

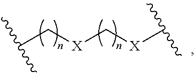

wherein each instance of n is independently an integer from 1-8, inclusive, and X is —O—, —NR^{A}, —S—, —C(═O)—, —NR^{A}C(═O)—, —C(═O)NR^{A}—, —OC(═O)—, or —C(═O)O—. In some embodiments, the linker, L, is

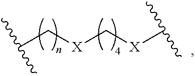

wherein n is independently an integer from 1-8, inclusive, and X is —O—, —NR^{A}—, —S—, —C(═O)—, —NR^{A}C(═O)—, —C(═O)NR^{A}-, —OC(═O)—, or —C(═O)O—. In some embodiments, the linker, L,

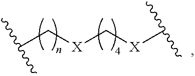

wherein each instance of n is independently selected from 2, 4, and 5, and X is —O—, —NR^{A}—, —S—, —C(═O)—, —NR^{A}C(═O)—, —C(═O)NR^{A}—, —OC(═O)—, or —C(═O)O—.

In some embodiments, the linker,

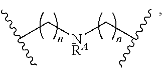

wherein each instance of n is independently an integer from 1-8, inclusive. In some embodiments, the linker, L, is

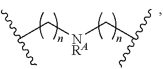

wherein each instance of n is independently selected from 2, 4, and 5. In some embodiments, the linker, L, is

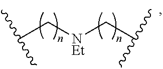

wherein each instance of n is independently selected from 2, 4, and 5. In some embodiments, the linker, L, is

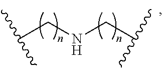

wherein each instance of n is independently selected from 2, 4, and 5. In some embodiments, the linker, L, is

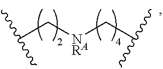

In some embodiments, the linker, L, is

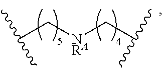

some embodiments, the linker, L, is

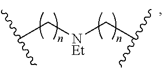

wherein each instance of n is independently an integer from 1-8, inclusive. In some embodiments, the linker, L, is

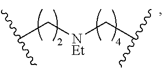

In some embodiments, the linker, L, is

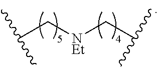

In some embodiments, the linker, L, is

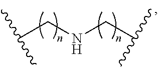

wherein each instance of n is independently an integer from 1-8, inclusive. In some embodiments, the linker, L, is

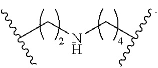

In some embodiments, the linker, L, is

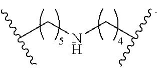

In some embodiments, the linker, L, is

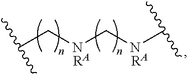

wherein each instance of n is independently an integer from 1-8, inclusive. In some embodiments, the linker, L, is

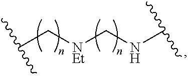

wherein each instance of n is independently an integer from 1-8, inclusive. In some embodiments, the linker, L,

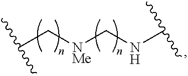

wherein each instance of n is independently an integer from 1-8, inclusive. In some embodiments, the linker, L, is

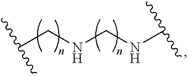

wherein each instance of n is independently an integer from 1-8, inclusive. In some embodiments, the linker, L, is

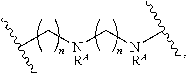

wherein each instance of n is independently selected from 2, 4, and 5. In some embodiments, the linker, L, is

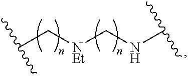

wherein each instance of n is independently selected from 2, 4, and 5. In some embodiments, the linker, L, is

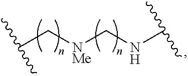

wherein each instance of n is independently selected from 2, 4, and 5. In some embodiments, the linker, L, is

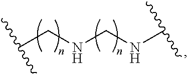

wherein each instance of n is independently selected from 2, 4, and 5. In some embodiments, the linker, L, is

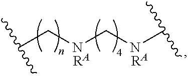

wherein each instance of n is independently selected from 2, 4, and 5. In some embodiments, the linker, L, is

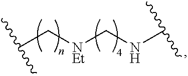

wherein each instance of n is independently selected from 2, 4, and 5. In some embodiments, the linker, L, is

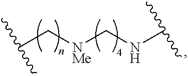

wherein each instance of n is independently selected from 2, 4, and 5. In some embodiments, the linker, L, is

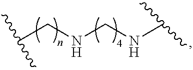

wherein each instance of n is independently selected from 2, 4, and 5.

### Sugar (R¹)

The compounds of Formula (I), and subgenera thereof, comprise R¹ wherein R¹ is substituted or unsubstituted sugar, substituted or unsubstituted amino sugar, or substituted or unsubstituted deoxy sugar. In some embodiments, R¹ is substituted or unsubstituted sugar. In some embodiments, R¹ is substituted or unsubstituted amino sugar. In some embodiments, R¹ is substituted or unsubstituted deoxy sugar. In some embodiments, the sugar is a monosaccharide. In certain embodiments, the sugar is allose, altose, arabinose, dextrose, erythrose, erythrulose, fructose, galactose, glucose, gulose, idose, lactose, lyxose, maltose, mannose, psicose, ribose, ribulose, sorbose, sucrose, tagatose, talose, threose, xylose, or derivative thereof. In some embodiments, the amino sugar is N-acetylglucosamine, N-acetylgalactosamine, N-acetylmannosamine, glucuronic acid, chondroitin sulfate, or hyaluronic acid. In some embodiments, the amino sugar is N-Acetylmannosamine. In some embodiments, the deoxy sugar is fucose, fuculose, or rhamnose. In some embodiments R¹ is mannose or fucose. In certain embodiments, the sugar is allose, altose, arabinose, dextrose, erythrose, erythrulose, fructose, galactose, glucose, gulose, idose, lactose, lyxose, maltose, mannose, psicose, ribose, ribulose, sorbose, sucrose, tagatose, talose, threose, or xylose. In some embodiments, the sugar is mannose, sucrose, or glucose. In some embodiments, R¹ is substituted or unsubstituted mannose, substituted or unsubstituted sucrose, or substituted or unsubstituted glucose. In some embodiments, R¹ is substituted or unsubstituted allose. In some embodiments, R¹ is substituted or unsubstituted altose. In some embodiments, R¹ is substituted or unsubstituted arabinose. In some embodiments, R¹ is substituted or unsubstituted dextrose. In some embodiments, R¹ is substituted or unsubstituted erythrose. In some embodiments, R¹ is substituted or unsubstituted erythrulose. In some embodiments, R¹ is substituted or unsubstituted fructose. In some embodiments, R¹ is substituted or unsubstituted galactose. In some embodiments, R¹ is substituted or unsubstituted glucose. In some embodiments, R¹ is substituted or unsubstituted gulose. In some embodiments, R¹ is substituted or unsubstituted idose. In some embodiments, R¹ is substituted or unsubstituted lactose. In some embodiments, R¹ is substituted or unsubstituted lyxose. In some embodiments, R¹ is substituted or unsubstituted maltose. In some embodiments, R¹ is substituted or unsubstituted mannose. In some embodiments, R¹ is substituted or unsubstituted psicose. In some embodiments, R¹ is substituted or unsubstituted ribose. In some embodiments, R¹ is substituted or unsubstituted ribulose. In some embodiments, R¹ is substituted or unsubstituted sorbose. In some embodiments, R¹ is substituted or unsubstituted sucrose. In some embodiments, R¹ is substituted or unsubstituted tagatose. In some embodiments, R¹ is substituted or unsubstituted talose. In some embodiments, R¹ is substituted or unsubstituted threose. In some embodiments, R¹ is substituted or unsubstituted xylose.

In some embodiments, R¹ is a substituted sugar. In some embodiments, R¹ is substituted allose. In some embodiments, R¹ is substituted altose. In some embodiments, R¹ is substituted arabinose. In some embodiments, R¹ is substituted dextrose. In some embodiments, R¹ is substituted erythrose. In some embodiments, R¹ is substituted erythrulose. In some embodiments, R¹ is substituted fructose. In some embodiments, R¹ is substituted galactose. In some embodiments, R¹ is substituted glucose. In some embodiments, R¹ is substituted gulose. In some embodiments, R¹ is substituted idose. In some embodiments, R¹ is substituted lactose. In some embodiments, R¹ is substituted lyxose. In some embodiments, R¹ is substituted maltose. In some embodiments, R¹ is substituted mannose. In some embodiments, R¹ is substituted psicose. In some embodiments, R¹ is substituted ribose. In some embodiments, R¹ is substituted ribulose. In some embodiments, R¹ is substituted sorbose. In some embodiments, R¹ is substituted sucrose. In some embodiments, R¹ is substituted tagatose. In some embodiments, R¹ is substituted talose. In some embodiments, R¹ is substituted threose. In some embodiments, R¹ is substituted xylose.

In some embodiments, the substituent on the sugar is C₁₋₆ alkyl, —C(═O)(C₁₋₆ alkyl), an oxygen protecting group, —S(═O)₂(OH), —S(═O)₂(OH)₂, or —P(═O)(OH)₂. In some embodiments, R¹ is substituted xylose. In some embodiments, the substituent on the sugar is methyl, —C(═O)(C₁₋₆ alkyl), an oxygen protecting group, —S(═O)₂(OH), —S(═O)₂(OH)₂, or —P(═O)(OH)₂. In some embodiments, the substituent on the sugar is —S(═O)₂(OH), —S(═O)₂(OH)₂, or —P(═O)(OH)₂. In some embodiments the substituent on the sugar is —P(═O)(OH)₂.

In some embodiments, R¹ is unsubstituted allose. In some embodiments, R¹ is unsubstituted altose. In some embodiments, R¹ is unsubstituted arabinose. In some embodiments, R¹ is unsubstituted dextrose. In some embodiments, R¹ is unsubstituted erythrose. In some embodiments, R¹ is unsubstituted erythrulose. In some embodiments, R¹ is unsubstituted fructose. In some embodiments, R¹ is unsubstituted galactose. In some embodiments, R¹ is unsubstituted glucose. In some embodiments, R¹ is unsubstituted gulose. In some embodiments, R¹ is unsubstituted idose. In some embodiments, R¹ is unsubstituted lactose. In some embodiments, R¹ is unsubstituted lyxose. In some embodiments, R¹ is unsubstituted maltose. In some embodiments, R¹ is unsubstituted mannose. In some embodiments, R¹ is unsubstituted psicose. In some embodiments, R¹ is unsubstituted ribose. In some embodiments, R¹ is unsubstituted ribulose. In some embodiments, R¹ is unsubstituted sorbose. In some embodiments, R¹ is unsubstituted sucrose. In some embodiments, R¹ is unsubstituted tagatose. In some embodiments, R¹ is unsubstituted talose. In some embodiments, R¹ is unsubstituted threose. In some embodiments, R¹ is unsubstituted xylose.

In some embodiments, the sugar is selected from the group consisting of:

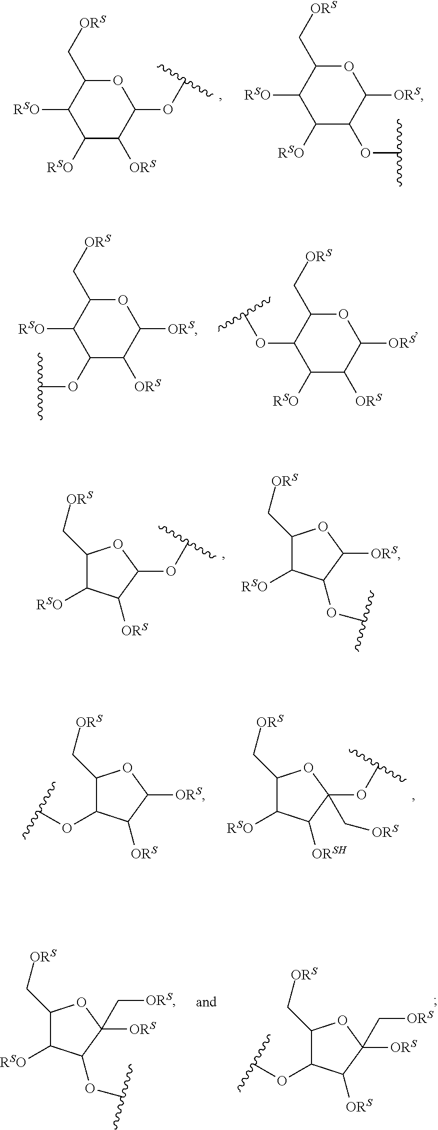

wherein R^{S} is selected from the group consisting of hydrogen, C₁₋₆ alkyl, —C(═O)(C₁₋₆ alkyl), an oxygen protecting group, —S(═O)₂(OH), —S(═O)₂(OH)₂, and —P(═O)(OH)₂, and salts thereof.

In some embodiments, the sugar is selected from the group consisting of:

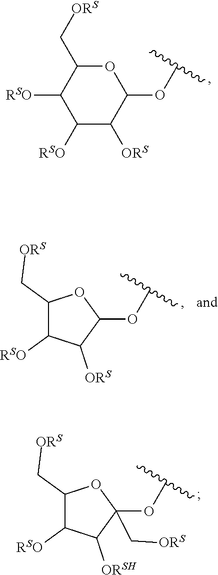

wherein R^{S} is selected from the group consisting of hydrogen, C₁₋₆ alkyl, —C(═O)(C₁₋₆ alkyl), an oxygen protecting group, —S(═O)₂(OH), —S(═O)₂(OH)₂, and —P(═O)(OH)₂, and salts thereof.

In some embodiments, the sugar is:

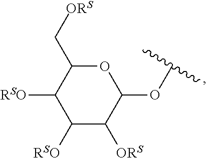

wherein R^{S} is selected from the group consisting of hydrogen, C₁₋₆ alkyl, —C(═O)(C₁₋₆ alkyl), an oxygen protecting group, —S(═O)₂(OH), —S(═O)₂(OH)₂, and —P(═O)(OH)₂, and salts thereof.

In certain embodiments, the sugar is:

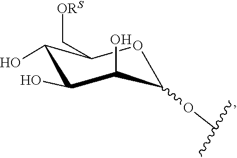

wherein R^{S} is selected from the group consisting of hydrogen, C₁₋₆ alkyl, —C(═O)(C₁₋₆ alkyl), an oxygen protecting group, —S(═O)₂(OH), —S(═O)₂(OH)₂, and —P(═O)(OH)₂, and salts thereof.

In some embodiments, R^{S} is —H, —SO₃H, or —PO₃H₂. In some embodiments, R^{S} is —H or —SO₃H. In some embodiments, R^{S} is —H. In some embodiments, R^{S} is —SO₃H. In some embodiments, R^{S} is —PO₃H₂.

As provided herein, R¹, the sugar moiety of Formula (I) may comprise R^{S}. In some embodiments, R^{S} is selected from the group consisting of hydrogen, C₁₋₆ alkyl, —C(═O)(C₁₋₆ alkyl), an oxygen protecting group, —S(═O)₂(OH), —S(═O)₂(OH)₂, and —P(═O)(OH)₂, and salts thereof. In some embodiments, R^{S} is selected from the group consisting of hydrogen, C₁₋₆ alkyl, —S(═O)₂(OH), —S(═O)₂(OH)₂, and —P(═O)(OH)₂. In some embodiments, R^{S} is selected from the group consisting of hydrogen, C₁₋₆ alkyl, and —P(═O)(OH)₂. In some embodiments, R^{S} is selected hydrogen. In some embodiments, R^{S} is C₁₋₆ alkyl. In some embodiments, R^{S} is —P(═O)(OH)₂.

In some embodiments, the sugar is selected from:

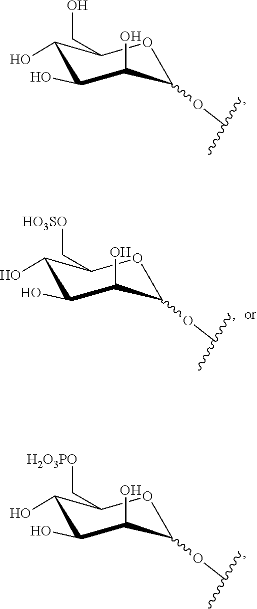

or salt thereof.

In some embodiments, the sugar is selected from:

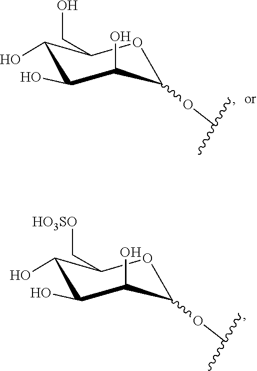

or salt thereof.

In some embodiments, R¹ binds to a receptor expressed by a cell. In some embodiments the receptor is a glycan-binding receptor. In some embodiments, the receptor is a sugar-binding receptor. In some embodiments, the cell is a cell that expresses syndecan, α-dystoglycan CD44, CD44, glypicans, NG2, RPTP-σ, GPI-brevican, RHAMM, Toll-like, IGF-II, mannose, or mannose-6-phosphate receptors. In some embodiments, the cell is a cell that expresses syndecan, CD44, glypicans, NG2, RPTP-σ, GPI-brevican, RHAMM, Toll-like, IGF-II, mannose, or mannose-6-phosphate receptors. In some embodiments, the receptor is IGF-II (insulin-like growth factor 2 receptor). In certain embodiments, the receptor is a mannose receptor. In certain embodiments, the receptor is mannose-6-phosphate receptor.

In some embodiments, the compound of Formula (I) is of the formula:

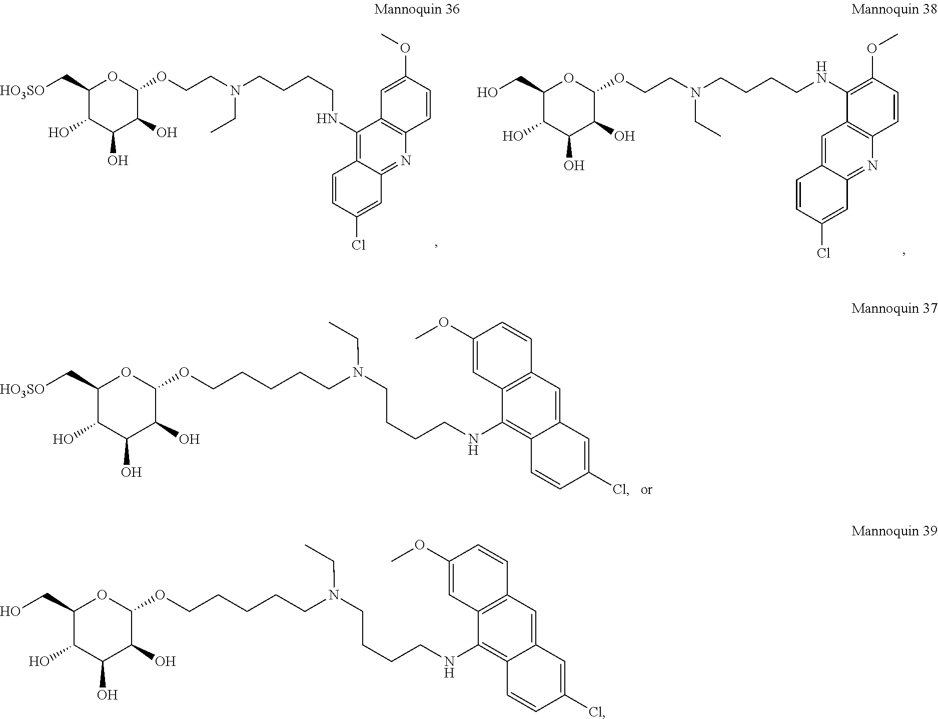

or a pharmaceutically acceptable salt thereof.

In some embodiments, the compound of Formula (I) is of the formula:

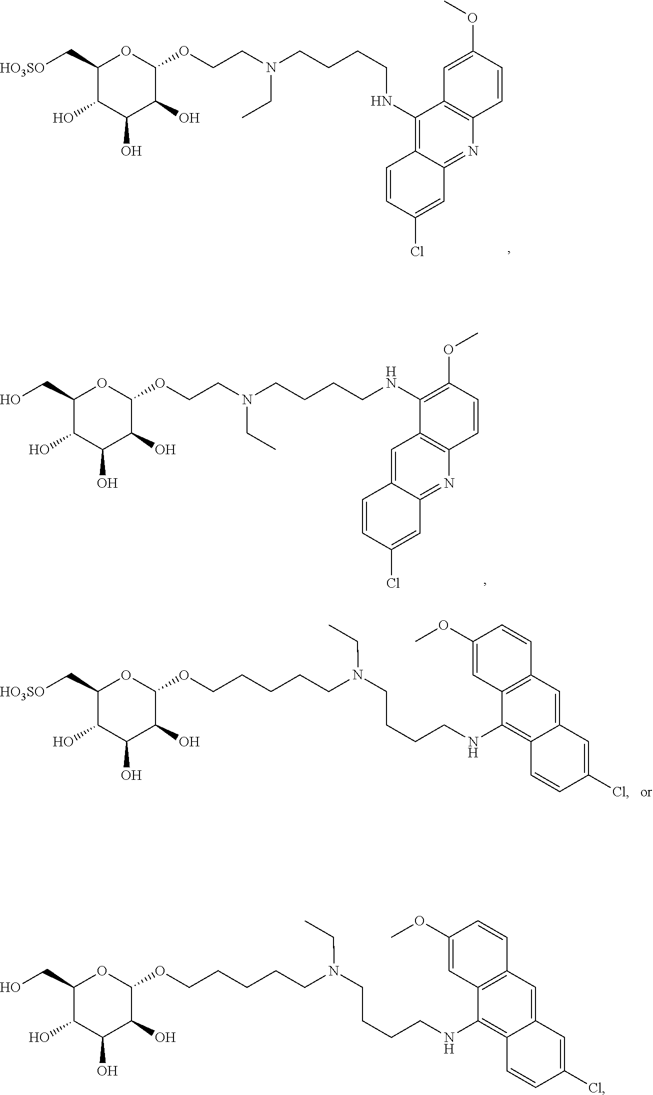

or a pharmaceutically acceptable salt thereof,

### DNA

In certain embodiments, the present disclosure provides compositions comprising a compound of Formula (I) and a component to deliver to a subject (e.g., a cell of a subject). In certain embodiments, the present disclosure provides compositions comprising a compound of Formula (I) and genetic material.

In certain embodiments, the present disclosure provides compositions comprising a compound of Formula (I) and DNA. In certain embodiments, the present disclosure provides compositions comprising a compound of Formula (I), DNA, and a pharmaceutically acceptable excipient, Exemplary DNAs include single-stranded DNA (ssDNA), double-stranded DNA (dsDNA), plasmid DNA (pDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), provirus, lysogen, repetitive DNA, satellite DNA, DNA minicircle, and viral DNA, Exemplary RNAs include single-stranded RNA (ssRNA), double-stranded RNA (dsRNA), small interfering RNA (siRNA), messenger RNA (mRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, and viral satellite RNA. In some embodiments, the DNA is single-stranded DNA (ssDNA), double-stranded DNA (dsDNA), plasmid DNA (pDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), provirus, lysogen, repetitive DNA, satellite DNA, DNA minicircle, or viral DNA. In some embodiments, the DNA is in the form of a plasmid, cosmid, bacterial artificial chromosome, yeast artificial chromosome, natural chromosome, bacteriophage, and virus. In some embodiments, the DNA is double-stranded DNA. In some embodiments, the DNA is a plasmid. In some embodiments, the DNA is a DNA minicircle.

In certain embodiments, the present disclosure provides compositions comprising a compound of Formula (I) and a plasmid. In certain embodiments, the present disclosure provides compositions comprising a compound of Formula (I), a plasmid, and a pharmaceutically acceptable excipient. In some embodiments, the plasmid encodes a transgene. In some embodiments, the transgene encodes infusible or injectable therapeutic proteins, enzymes, enzyme cofactors, hormones, blood or blood coagulation factors, cytokines and interferons, growth factors, adipokines, lysozyme, oxidoreductases, transferases, hydrolases, lyases, isomerases, asparaginases, uricases, glycosidases, proteases, nucleases, collagenases, hyaluronidases, heparinases, heparanases, kinases, phosphatases, lysins, ligases, imiglucerase, a-galactosidase A (a-gal A), acid a-glucosidase (GAA), arylsulfatase B, gonadotropins, thyroid-stimulating hormone, melanocortins, pituitary hormones, vasopressin, oxytocin, growth hormones, prolactin, orexins, natriuretic hormones, parathyroid hormone, calcitonins, erythropoietin, and pancreatic hormones, Factor I (fibrinogen), Factor II (prothrombin), tissue factor, Factor V (proaccelerin, labile factor), Factor VII (stable factor, proconvertin), Factor VIII (antihemophilic globulin), Factor IX (Christmas factor or plasma thromboplastin component), Factor X (Stuart-Prower factor), Factor Xa, Factor XI, Factor XII (Hageman factor), Factor XIII (fibrin-stabilizing factor), von Willebrand factor, von Heldebrant Factor, prekallikrein (Fletcher factor), high-molecular weight kininogen (HMWK) (Fitzgerald factor), fibronectin, fibrin, thrombin, antithrombin, such as antithrombin III, heparin cofactor 11, protein C, protein S, protein Z, protein Z-related protease inhibitot (ZPI), plasminogen, alpha 2-antiplasmin, tissue plasminogen activator (tPA), urokinase, plasminogen activator inhibitor-1 (PAI1), plasminogen activator inhibitor-2 (PAI2), cancer procoagulant, and epoetin alfa (Epogen, Procrit), lymphokines, interleukins, and chemokines, type 1 cytokines, such as IFN-γ, TGF-β, Adrenomedullin (AM), Angiopoietin (Ang), Autocrine motility factor, Bone morphogenetic proteins (BMPs), Brain-derived neurotrophic factor (BDNF), Epidermal growth factor (EGF), Erythropoietin (EPO), Fibroblast growth factor (FGF), Glial cell line-derived neurotrophic factor (GDNF), Granulocyte colony-stimulating factor (G-CSF), Granulocyte macrophage colony-stimulating factor (GM-CSF), Growth differentiation factor-9 (GDF9), Hepatocyte growth factor (HGF), Hepatoma-derived growth factor (HDGF). Insulin-like growth factor (IGF), Migration-stimulating factor, Myostatin (GDF-8), Nerve growth factor (NGF) and other neurotrophins, Platelet-derived growth factor (PDGF), Thrombopoietin (TPO), Transforming growth factor alpha (TGF-α), Transforming growth factor beta (TGF-β), Tumour necrosis factor-alpha (TNF-α), Vascular endothelial growth factor (VEGF), Wnt Signaling Pathway, placental growth factor (PlGF), [(Foetal Bovine Somatotrophin)](FBS), IL-1, IL-2, IL-3, IL-4, IL-5, IL-6, and IL-7, receptors, signaling proteins, cytoskeletal proteins, scaffold proteins, transcription factors, structural proteins, membrane proteins, cytosolic proteins, binding proteins, nuclear proteins, secreted proteins, Golgi proteins, endoplasmic reticulum proteins, mitochondrial proteins, and vesicular proteins. In some embodiments, the transgene is a gene for which a disease is associated with reduced expression, lack of expression or dysfunction of the native gene (e.g., glucose-6-phosphatase, associated with glycogen storage deficiency type 1A; phosphoenolpyruvate-carboxykinase, associated with Pepck deficiency; galactose-1 phosphate uridyl transferase, associated with galactosemia; phenylalanine hydroxylase, associated with phenylketonuria: branched chain alpha-ketoacid dehydrogenase, associated with Maple syrup urine disease; fumarylacetoacetate hydrolase, associated with tyrosinemia type 1; methylmalonyl-CoA mutase, associated with methylmalonic acidemia; medium chain acyl CoA dehydrogenase, associated with medium chain acetyl CoA deficiency; omithine transcarbamylase, associated with omithine transcarbamylase deficiency; argininosuccinic acid synthetase, associated with citrullinemia; low density lipoprotein receptor protein, associated with familial hypercholesterolemia; UDP-glucouronosyltransferase, associated with Crigler-Najjar disease: adenosine deaminase, associated with severe combined immunodeficiency disease; hypoxanthine guanine phosphoribosyl transferase, associated with Gout and Lesch-Nyan syndrome; biotinidase, associated with biotinidase deficiency; beta-glucocerebrosidase, associated with Gaucher disease; beta-glucuronidase, associated with Sly syndrome; peroxisome membrane protein 70 kDa, associated with Zellweger syndrome; porphobilinogen deaminase, associated with acute intermittent porphyria; alpha-1 antitrypsin for treatment of alpha-1 antitrypsin deficiency (emphysema); erythropoietin for treatment of anemia due to thalassemia or to renal failure; vascular endothelial growth factor, angiopoietin-1, and fibroblast growth factor for the treatment of ischemic diseases; thrombomodulin and tissue factor pathway inhibitor for the treatment of occluded blood vessels as seen in, for example, atherosclerosis, thrombosis, or embolisms; aromatic amino acid decarboxylase (AADC), and tyrosine hydroxylase (TH) for the treatment of Parkinson's disease; the beta adrenergic receptor, anti-sense to, or a mutant form of, phospholamban, the sarco(endo)plasmic reticulum adenosine triphosphatase-2 (SERCA2), and the cardiac adenylyl cyclase for the treatment of congestive heart failure: a tumor suppressor gene such as p53 for the treatment of various cancers; a cytokine such as one of the various interleukins for the treatment of inflammatory and immune disorders and cancers; dystrophin or minidystrophin and utrophin or miniutrophin for the treatment of muscular dystrophies; and, insulin for the treatment of diabetes). In some embodiments, the transgene encodes a protein for which a disease is associated with reduced expression, lack of expression or dysfunction of the native gene (e.g., a-galactosidase, acid-glucosidase, adiopokines, adiponectin, alglucosidase alfa, anti-thrombin, ApoAV, ApoCII, apolipoprotein A-I (APOA1), arylsulfatase A, arylsulfatase B, ATP-binding cassette transporter A1 (ABCA1), ABCD1, CCR5 receptor, erythropoietin, Factor VIII, Factor VII, Factor IX, Factor V, fetal hemoglobin, beta-globin, GPI-anchored HDL-binding protein (GPI-HBP) I, growth hormone, hepatocyte growth factor, imiglucerase, lecithin-cholesterol acyltransferase (LCAT), leptin, LDL receptor, lipase maturation factor (LMF) 1, lipoprotein lipase, lysozyme, nicotinamide dinucleotide phosphate (NADPH) oxidase, Rab escort protein-1 (REP-1), retinal degeneration slow (RDS), retinal pigment epithelium-specific 65 (RPE65), rhodopsin, T cell receptor alpha or beta chains, thrombopoeitin, tyrosine hydroxylase, VEGF, von heldebrant factor, von willebrand factor, and X-linked inhibitor of apoptosis (XIAP)). In some embodiments, the transgene encodes a CRISPR-Cas enzyme (e.g., Cas9, Cpf1 (Cas12a), CasX (Cas12e), C2c1 (Cas12b1), Cas12b2, C2c3 (Cas12c), CasY (Cas12d), C2c4, C2c8, C2c5, C2c10, C2c9, C2c2 (Cas13a), Cas13d, C2c7 (Cas13c), C₂c6 (Cas13b)). In some embodiments, the transgene encodes a transcription factor (e.g., Sp1, NF1, CCAAT, GATA, HNF, PIT-1, MyoD, Myf5, Hox, Winged Helix, SREBP, p53, CREB, AP-1, Mef2, STAT, R-SMAD, NF-κB, Notch, TUBBY, NFAT). In some embodiments, the plasmid encodes a cytokine, peptide hormone, glycoprotein hormone, or antibody. In some embodiments, the plasmid encodes a cytokine. In some embodiments, the cytokine is IL-10, IL-36RN, IL-33, or IL-37. In some embodiments, the cytokine binds to IL-1ORI. In certain embodiments, the plasmid encodes a glycoprotein cytokine. In some embodiments, the plasmid encodes erythropoietin. In certain embodiments, the plasmid encodes a glycoprotein hormone (e.g., follicle-stimulating hormone, luteinizing hormone, thyroid-stimulating hormone, and human chorionic gonadotropin). In some embodiments, the plasmid encodes an antibody. In certain embodiments, the plasmid is multicistronic. In certain embodiments, the antibody is an anti-PD-1 antibody, an anti-PD-LI antibody, an anti-CTLA-4 antibody, an anti-TIM3 antibody, an anti-OX40 antibody, an anti-GITR antibody, an anti-LAG-3 antibody, an anti-CD137 antibody, an anti-CD27 antibody, an anti-CD28 antibody, an anti-CD28H antibody, an anti-CD30 antibody, an anti-CD39 antibody, an anti-CD40 antibody, an anti-CD47 antibody, an anti-CD48 antibody, an anti-CD70 antibody, an anti-CD73 antibody, an anti-CD96 antibody, an anti-CD160 antibody, an anti-CD200 antibody, an anti-CD244 antibody, an anti-ICOS antibody, an anti-TNFRSF25 antibody, an anti-TMIGD2 antibody, an anti-DNAM 1 antibody, an anti-BTLA antibody, an anti-LIGHT antibody, an anti-TIGIT antibody, an anti-VISTA antibody, an anti-HVEM antibody, an anti-Siglec antibody, an anti-GAL1 antibody, an anti-GAL3 antibody, an anti-GAL9 antibody, an anti-BTNL2 (butrophylins) antibody, an anti-B7-H3 antibody, an anti-B7-H4 antibody, an anti-B7-H5 antibody, an anti-B7-H6 antibody, an anti-KIR antibody, an anti-LIR antibody, an anti-ILT antibody, an anti-MICA antibody, an anti-MICB antibody, an anti-NKG2D antibody, an anti-NKG2A antibody, an anti-TGFβ antibody, an anti-TGFβR antibody, an anti-CXCR4 antibody, an anti-CXCL12 antibody, an anti-CCL2 antibody, an anti-IL-10 antibody, an anti-IL-13 antibody, an anti-IL-23 antibody, an anti-phosphatidylserine antibody, an anti-neuropilin antibody, an anti-GalCer antibody, an anti-HER² antibody, an anti-VEGFA antibody, an anti-VEGFR antibody, an anti-EGFR antibody, or an anti-Tie2 antibody. In some embodiments, the plasmid encodes adecatumumab (MT201), afutuzumab, alemtuzumab (CAMPATH), bavituximab, belimumab, bevacizumab (AVASTIN), brentuximab (ADCETRIS), cantuzumab, cetuximab (ERBITUX), citatuzumab, cixutumumab, conatumumab, dacetuzumab, elotuzumab, etaracizumab, farletuzumab, figitumumab, gemtuzumab, ibritumomab, inotuzumab (CMC-533), ipilimumab (YERVOY), iratumumab, labetuzumab, lexatumumab, lintuzumab, lucatumumab, mapatumumab, matuzumab, milatuzumab, necitumumab, nimotuzumab, ofatumumab (ARZERRA), olaratumab, oportuzumab, panitumumab (VECTIBIX), pertuzumab (PERJETA), pritumumab, rituximab (RITUXAN), robatumumab, sibrotuzumab, siltuximab, tacatuzumab, tigatuzumab, tositumomab (BEXXAR), trastuzumab (HERCEPTIN), tucotuzumab, veltuzumab, votumumab, zalutumumab, pembrolizumab, nivolumab, pidilizumab, ipilimumab, tremelimumab, durvalumab, atezolizumab, avelumab, PF-06801591, utomilumab, PDR001, PBF-509, MGB453, LAG525, AMP-224, INCSHR1210, INCAGN1876, INCAGN1949, samalizumab, PF-05082566, urelumab, lirilumab, lulizumab, BMS-936559, BMS-936561, BMS-986004, BMS-986012, BMS-986016, BMS-986178, IMP321, IPH2101, IPH2201, varilumab, ulocuplumab, monalizumab, MEDI0562, MEDI0680, MEDI1873, MEDI6383, MEDI6469, MEDI9447, AMG228, AMG820, CC-90002, CDX-1127, CGEN15001T, CGEN15022, CGEN15029, CGEN15049, CGEN15027, CGEN15052, CGEN15092, CX-072, CX-2009, CP-870893, lucatumumab, dacetuzumab, Chi Lob 7/4, RG6058, RG7686, RG7876, RG7888, TRX518, MK-4166, MGA271, IMC-CS4, emactuzumab, trastuzumab, pertuzumab, obinutuzumab, cabiralizumab, margetuximab, enoblituzumab, mogamulizumab, panitumumab, carlumab, bevacizumab, rituximab, cetuximab, fresolimumab, FAZ053, TSR022, MBG453, REGN2810, REGN3767, MOXR0916, PF-04518600, R07009789, BMS986156, GWN323, JTX-2011, NKTR-214, GSK3174998, DS-8273a, NIS793, BGB-A317, abciximab, adalimumab, basiliximab, blinatumomab, denosumab, or ranibizumab. In some embodiments, the plasmid encodes denosumab or ranibizumab. In certain embodiments, the plasmid encodes a cytokine, glycoprotein hormone, or antibody. In some embodiments, the plasmid encodes the amino acid sequence of a cytokine, glycoprotein hormone, or antibody 100% identical to a wild type sequence. In some embodiments, the wild type sequence is a human wild type sequence. In some embodiments, the wild type sequence is a non-human wild type sequence. In some embodiments, the plasmid encodes a cytokine or glycoprotein hormone at least 99%, 98%, 95%, 90%, 85%, and 80% identical to a wild type sequence. In some embodiments, the plasmid encodes a cytokine or glycoprotein hormone at least 99% identical to the wild type sequence. In some embodiments, the plasmid encodes a cytokine or glycoprotein hormone at least 98% identical to a wild type sequence. In some embodiments, the plasmid encodes a cytokine or glycoprotein hormone at least 95% identical to a wild type sequence. In some embodiments, the plasmid encodes a cytokine or glycoprotein hormone at least 90% identical to a wild type sequence. In some embodiments, the plasmid encodes cytokine or glycoprotein hormone at least 85% identical to a wild type sequence. In some embodiments, the plasmid encodes a cytokine or glycoprotein hormone at least 80% identical to a wild type sequence. In some embodiments, the plasmid encodes an antibody at least 99%, 98%, 95%, 90%, 85%, and 80% identical to a parent sequence. In some embodiments, the plasmid encodes an antibody at least 99% identical to the wild type sequence. In some embodiments, the plasmid encodes an antibody at least 98% identical to a parent sequence. In some embodiments, the plasmid encodes an antibody at least 95% identical to a parent sequence. In some embodiments, the plasmid encodes an antibody at least 90% identical to a parent sequence. In some embodiments, the plasmid encodes an antibody at least 85% identical to a parent sequence. In some embodiments, the plasmid encodes an antibody at least 80% identical to a parent sequence.

In some embodiments, the composition comprises a compound of Formula (I) and a plasmid encoding a IL-10 polypeptide. In some embodiments, the IL-10 polypeptide is wild type IL-10. In some embodiments, the IL-10 polypeptide comprises a mutation. In some embodiments, the mutation is within the portion of the IL-10 polypeptide comprising: LRRCHRFLPCENKSK (SEQ ID NO: 26). In some embodiments, the mutation is within the portion of the IL-10 polypeptide: LRRCHRFLPCENKSK (SEQ ID NO: 26) of any one of SEQ ID NO: 3 through SEQ ID NO: 22, or any other IL-10 peptide. In certain embodiments, the mutation is within the portion of the IL-10 polypeptide: LRRCHRFLPCENKSK (SEQ ID NO: 26) of any one of SEQ ID NO: 3 through SEQ ID NO: 22. In some embodiments, the mutation is within the portion of the IL-10 polypeptide: LRRCHRFLPCENKSK (SEQ ID NO: 26) of SEQ ID NO: 3, SEQ ID NO: 7, SEQ ID NO: 8, or SEQ ID NO: 21. In certain embodiments, the mutation is within the portion of the IL-10 polypeptide: LRRCHRFLPCENKSK (SEQ ID NO: 26) of SEQ ID NO: 4 through SEQ ID NO: 6, SEQ ID NO: 9, SEQ ID NO: 11-19, SEQ ID NO: 22-25. In some embodiments, the mutation is a substitution of serine for phenylalanine at: (i) position 129 of any one of SEQ ID NO: 3 through SEQ ID NO: 18 or SEQ ID NO: 23-25; (ii) position 130 of SEQ ID NO: 19 or SEQ ID NO: 20; (iii) position 132 of SEQ ID NO: 21; or (iv) position 171 of SEQ ID NO: 22. In some embodiments, the mutation is a substitution of serine for phenylalanine at position 129 of any one of SEQ ID NO: 3 through SEQ ID NO: 18 or SEQ ID NO: 23-25. In some embodiments, the mutation is a substitution of serine for phenylalanine at position 130 of SEQ ID NO: 19 or SEQ ID NO: 20. In some embodiments, the mutation is a substitution of serine for phenylalanine at position 132 of SEQ ID NO: 21. In some embodiments, the mutation is a substitution of serine for phenylalanine at position 171 of SEQ ID NO: 22. In some embodiments, the plasmid encodes a polypeptide at least 99%, 98%, 95%, 90%, 85%, and 80% identical to any one of SEQ ID NO: 3 through SEQ ID NO: 25.

In some embodiments, the plasmid encodes the amino acid sequence of SEQ ID NO: 1. In some embodiments, the plasmid encodes a IL-10 polypeptide at least 99%, 98%, 95%, 90%, 85%, and 80% identical to SEQ ID NO: 1. In some embodiments, the plasmid encodes a IL-10 polypeptide comprising the amino acid sequence of SEQ ID NO: 1. In certain embodiments, the plasmid encodes a polypeptide at least 99% identical to SEQ ID NO: 1. In some embodiments, the plasmid encodes a polypeptide at least 98% identical to SEQ ID NO: 1. In certain embodiments, the plasmid encodes a polypeptide at least 95% identical to SEQ ID NO: 1. In some embodiments, the plasmid encodes a polypeptide at least 90% identical to SEQ ID NO: 1. In certain embodiments, the plasmid encodes a polypeptide at least 85% identical to SEQ ID NO: 1. In some embodiments, the plasmid encodes a polypeptide at least 80% identical to SEQ ID NO: 1.

In some embodiments, the plasmid comprises the nucleic acid sequence of XT-150. In some embodiments, the plasmid comprises the nucleic acid sequence of XT-151. In certain embodiments, the plasmid comprises the nucleic acid sequence of SEQ ID NO: 2. In some embodiments, the plasmid encodes a IL-10 polypeptide at least 99%, 98%, 95%, 90%, 85%, and 80% identical to SEQ ID NO: 2. In some embodiments, the plasmid comprises a nucleic acid at least 99% identical to SEQ ID NO: 2. In certain embodiments, the plasmid comprises a nucleic acid at least 98% identical to SEQ ID NO: 2. In some embodiments, the plasmid comprises a nucleic acid at least 95% identical to SEQ ID NO: 2. In certain embodiments, the plasmid comprises a nucleic acid at least 90% identical to SEQ ID NO: 2. In some embodiments, the plasmid comprises a nucleic acid at least 85% identical to SEQ ID NO: 2, In certain embodiments, the plasmid comprises a nucleic acid at least 80% identical to SEQ ID NO: 2.

In some embodiments, the plasmid encodes a protein with an amino acid sequence at least 100%, 99%, 98%, 95%, 90%, 85%, or 80% identical to an amino acid sequence native to a mammal. In certain embodiments, the plasmid encodes an amino sequence at least 100%, 99%, 98%, 95%, 90%, 85%, or 80% identical to an amino acid sequence native to a human or non-human animal. In certain embodiments, the plasmid encodes an amino sequence at least 100%, 99%, 98%, 95%, 90%, 85%, or 80% identical to an amino acid sequence native to a human. In certain embodiments, the plasmid encodes an amino sequence at least 100%, 99%, 98%, 95%, 90%, 85%, or 80% identical to an amino acid sequence native to a pet. In certain embodiments, the plasmid encodes an amino sequence at least 100%, 99%, 98%, 95%, 90%, 85%, or 80% identical to an amino acid sequence native to a farm animal. In certain embodiments, the plasmid encodes an amino sequence at least 100%, 99%, 98%, 95%, 90%, 85%, or 80% identical to an amino acid sequence native to a research animal. In certain embodiments, the plasmid encodes an amino sequence at least 100%, 99%, 98%, 95%, 90%, 85%, or 80% identical to an amino acid sequence native to a human, dog, horse, rats, mouse, cat, guinea pig, llama, gerbil, goat, sheep, ferret, rabbit, hamster, or monkey (e.g., chimp, macaques, marmoset, cynomolgus monkey, squirrel monkey). In certain embodiments, the plasmid encodes an amino sequence at least 100%, 99%, 98%, 95%, 90%, 85%, or 80% identical to an amino acid sequence native to a human, dog, horse, rats, mouse, cat, or dog. In certain embodiments, the plasmid encodes an amino sequence at least 100%, 99%, 98%, 95%, 90%, 85%, or 80% identical to an amino acid sequence native to a human, dog, horse, cat, or dog.

In some embodiments, the plasmid encodes a protein with an amino acid sequence at least 100%, 99%, 98%, 95%, 90%, 85%, or 80% identical to an amino acid sequence native to the mammalian subject. In certain embodiments, the plasmid encodes an amino sequence at least 100%, 99%, 98%, 95%, 90%, 85%, or 80% identical to an amino acid sequence native to the human or non-human animal subject. In certain embodiments, the plasmid encodes an amino sequence at least 100%, 99%, 98%, 95%, 90%, 85%, or 80% identical to an amino acid sequence native to the human subject. In certain embodiments, the plasmid encodes an amino sequence at least 100%, 99%, 98%, 95%, 90%, 85%, or 80% identical to an amino acid sequence native to the pet subject. In certain embodiments, the plasmid encodes an amino sequence at least 100%, 99%, 98%, 95%, 90%, 85%, or 80% identical to an amino acid sequence native to the farm animal subject. In certain embodiments, the plasmid encodes an amino sequence at least 100%, 99%, 98%, 95%, 90%, 85%, or 80% identical to an amino acid sequence native to the research animal subject. In certain embodiments, the plasmid encodes an amino sequence at least 100%, 99%, 98%, 95%, 90%, 85%, or 80% identical to an amino acid sequence native to the human, dog, horse, rats, mouse, cat, guinea pig, llama, gerbil, goat, sheep, ferret, rabbit, hamster, or monkey (e.g., chimp, macaques, marmoset, cynomolgus monkey, squirrel monkey) subject. In certain embodiments, the plasmid encodes an amino sequence at least 100%, 99%, 98%, 95%, 90%, 85%, or 80% identical to an amino acid sequence native to the human, dog, horse, rats, mouse, cat, or dog subject. In certain embodiments, the plasmid encodes an amino sequence at least 100%, 99%, 98%, 95%, 90%, 85%, or 80% identical to an amino acid sequence native to the human, dog, horse, cat, or dog subject,

IL-10^{F129S} mutant:
(SEQ ID NO: 1)
MHSSALLCCLVLLTGVRASPGQGTQSENSCTHFPGNLPNMLRDLRDAFSRVKTFFQMKDQ
LDNLLLKESLLEDFKGYLGCQALSEMIQFYLEEVMPQAENQDPDIKAHVNSLGENLKTLR
LRLRRCHRSLPCENKSKAVEQVKNAFNKLQEKGIYKAMSEFDIFINYIEAYMTMKIRN
XT-150 plasmid:
(SEQ ID NO: 2)
AGCTTTGCAGCCGGGCGTAATAGCGAAGAGGCCCGCACCGATCGCCCTTCCCAACAGTTG
CGCAGCCTGAATGGCGAATGGACGCGCCCTGTAGCGGCGCATTAAGCGCGGCGGGTGTGG
TGGTTACGCGCAGCGTGACCGCTACACTTGCCAGCGCCCTAGCGCCCGCTCCTTTCGCTT
TCTTCCCTTCCTTTCTCGCCACGTTCGCCGGCTTTCCCCGTCAAGCTCTAAATCGGGGGC
TCCCTTTAGGGTTCCGATTTAGTGCTTTACGGCACCTCGACCCCAAAAAACTTGATTAGG
GTGATGGTTCACGTAGTGGGCCATCGCCCTGATAGACGGTTTTTCGCCCTTTGACGTTGG
AGTCCACGTTCTTTAATAGTGGACTCTTGTTCCAAACTGGAACAACACTCAACCCTATCT
CGGTCTATTCTTTTGATTTATAAGGGATTTTGCCGATTTCGGCCTATTGGTTAAAAAATG
AGCTGATTTAACAAAAATTTAACGCGAATTTTAACAAAATATTAACGCTTACAATTTCCT
GATGCGGTATTTTCTCCTTACGCATCTGTGCGGTATTTCACACCGCATATGGTGCACTCT
CAGTACAATCTGCTCTGATGCCGCATAGTTAAGCCAGCCCCGACACCCGCCAACACCCGC
TGACGCGCCCTGACGGGCTTGTCTGCTCCCGGCATCCGCTTACAGACAAGCTGTGACCGT
CTCCGGGAGCTGCATGTGTCAGAGGTTTTCACCGTCATCACCGAAACGCGCGAGACGAAA
GGGCCTCGTGATACGCCTATTTTTATAGGTTAATGTCATGATAATAATGGTTTCTTAGAC
GTCAGGTGGCACTTTTCGGGGAAATGTGCGCGGAACCCCTATTTGTTTATTTTTCTAAAT
ACATTCAAATATGTATCCGCTCATGAGACAATAACCCTGATAAATGCTTCAATAATATTG
AAAAAGGAAGAGTAATTGAAACAAGATGGATTGCACGCAGGTTCTCCGGCCGCTTGGGTG
GAGAGGCTATTCGGCTATGACTGGGCACAACAGACAATCGGCTGCTCTGATGCCGCCGTG
TTCCGGCTGTCAGCGCAGGGGCGCCCGGTTCTTTTTGTCAAGACCGACCTGTCCGGTGCC
CTGAATGAACTGCAGGACGAGGCAGCGCGGCTATCGTGGCTGGCCACGACGGGCGTTCCT
TGCGCAGCTGTGCTCGACGTTGTCACTGAAGCGGGAAGGGACTGGCTGCTATTGGGCGAA
GTGCCGGGGCAGGATCTCCTGTCATCTCACCTTGCTCCTGCCGAGAAAGTATCCATCATG
GCTGATGCAATGCGGCGGCTGCATACGCTTGATCCGGCTACCTGCCCATTCGACCACCAA
GCGAAACATCGCATCGAGCGAGCACGTACTCGGATGGAAGCCGGTCTTGTCGATCAGGAT
GATCTGGACGAAGAGCATCAGGGGCTCGCGCCAGCCGAACTGTTCGCCAGGCTCAAGGCG
CGCATGCCCGACGGCGAGGATCTCGTCGTGACCCATGGCGATGCCTGCTTGCCGAATATC
ATGGTGGAAAATGGCCGCTTTTCTGGATTCATCGACTGTGGCCGGCTGGGTGTGGCGGAC
CGCTATCAGGACATAGCGTTGGCTACCCGTGATATTGCTGAAGAGCTTGGCGGCGAATGG
GCTGACCGCTTCCTCGTGCTTTACGGTATCGCCGCTCCCGATTCGCAGCGCATCGCCTTC
TATCGCCTTCTTGACGAGTTCTTCTGAGTAACCGTCAGACCAAGTTTACTCATATATACT
TTAGATTGATTTAAAACTTCATTTTTAATTTAAAAGGATCTAGGTGAAGATCCTTTTTGA
TAATCTCATGACCAAAATCCCTTAACGTGAGTTTTCGTTCCACTGAGCGTCAGACCCCGT
AGAAAAGATCAAAGGATCTTCTTGAGATCCTTTTTTTCTGCGCGTAATCTGCTGCTTGCA
AACAAAAAAACCACCGCTACCAGCGGTGGTTTGTTTGCCGGATCAAGAGCTACCAACTCT
TTTTCCGAAGGTAACTGGCTTCAGCAGAGCGCAGATACCAAATACTGTTCTTCTAGTGTA
GCCGTAGTTAGGCCACCACTTCAAGAACTCTGTAGCACCGCCTACATACCTCGCTCTGCT
AATCCTGTTACCAGTGGCTGCTGCCAGTGGCGATAAGTCGTGTCTTACCGGGTTGGACTC
AAGACGATAGTTACCGGATAAGGCGCAGCGGTCGGGCTGAACGGGGGGTTCGTGCACACA
GCCCAGCTTGGAGCGAACGACCTACACCGAACTGAGATACCTACAGCGTGAGCTATGAGA
AAGCGCCACGCTTCCCGAAGGGAGAAAGGCGGACAGGTATCCGGTAAGCGGCAGGGTCGG
AACAGGAGAGCGCACGAGGGAGCTTCCAGGGGGAAACGCCTGGTATCTTTATAGTCCTGT
CGGGTTTCGCCACCTCTGACTTGAGCGTCGATTTTTGTGATGCTCGTCAGGGGGGCGGAG
CCTATGGAAAAACGCCAGCAACGCGGCCTTTTTACGGTTCCTGGCCTTTTGCTGGCCTTT
TGCTCACATGTTCTTTCCTGCGTTATCCCCTGATTCTGTGGATAACCGTATTACCGCCTT
TGAGTGAGCTGATACCGCTCGCCGCAGCCGAACGACCGAGCGCAGCGAGTCAGTGAGCGA
GGAAGCGGAAGAGCGCCCAATACGCAAACCGCCTCTCCCCGCGCGTTGGCCGATTCATTA
ATGCAGGGCTGCAGCATGCCTGCAGGCAGCTGCGCGCTCGCTCGCTCACTGAGGCCGCCC
GGGCGTCGGGCGACCTTTGGTCGCCCGGCCTCAGTGAGCGAGCGAGCGCGCAGAGAGGGA
GTGGCCAACTCCATCACTAGGGGTTCCTGCGGCCGCACGCGTATCAATATTGGCCATTAG
CCATATTATTCATTGGTTATATAGCATAAATCAATATTGGCTATTGGCCATTGCATACGT
TGTATCTATATCATAATATGTACATTTATATTGGCTCATGTCCAATATGACCGCCATGTT
GGCATTGATTATTGACTAGTTATTAATAGTAATCAATTACGGGGTCATTAGTTCATAGCC
CATATATGGAGTTCCGCGTTACATAACTTACGGTAAATGGCCCGCCTGGCTGACCGCCCA
ACGACCCCCGCCCATTGACGTCAATAATGACGTATGTTCCCATAGTAACGCCAATAGGGA
CTTTCCATTGACGTCAATGGGTGGAGTATTTACGGTAAACTGCCCACTTGGCAGTACATC
AAGTGTATCATATGCCAAGTCCGCCCCCTATTGACGTCAATGACGGTAAATGGCCCGCCT
GGCATTATGCCCAGTACATGACCTTACGGGACTTTCCTACTTGGCAGTACATCTACGTAT
TAGTCATCGCTATTACCATGGTCGAGGTGAGCCCCACGTTCTGCTTCACTCTCCCCATCT
CCCCCCCCTCCCCACCCCCAATTTTGTATTTATTTATTTTTTAATTATTTTGTGCAGCGA
TGGGGGGGGGGGGGGGGGGGGGCGCGCGCCAGGCGGGGGGGGGGGGGGCGAGGGGGGGGG
GGGGCGAGGCGGAGAGGTGCGGCGGCAGCCAATCAGAGCGGCGCGCTCCGAAAGTTTCCT
TTTATGGCGAGGCGGCGGCGGCGGCGGCCCTATAAAAAGCGAAGCGCGCGGCGGGCGGGA
GTCGCTGCGCGCTGCCTTCGCCCCGTGCCCCGCTCCGCCGCCGCCTCGCGCCGCCCGCCC
CGGCTCTGACTGACCGCGTTACTCCCACAGGTGAGCGGGCGGGACGGCCCTTCTCCTCCG
GGCTGTAATTAGCGCTTGGTTTAATGACGGCTTGTTTCTTTTCTGTGGCTGCGTGAAAGC
CTTGAGGGGCTCCGGGAGGGCCCTTTGTGCGGGGGGAGCGGCTCGGGGGGTGCGTGCGTG
TGTGTGTGCGTGGGGAGCGCCGCGTGCGGCTCCGCGCTGCCCGGCGGCTGTGAGCGCTGC
GGGCGCGGCGCGGGGCTTTGTGCGCTCCGCAGTGTGCGCGAGGGGAGCGCGGCCGGGGGC
GGTGCCCCGCGGTGCGGGGGGGGCTGCGAGGGGAACAAAGGCTGCGTGCGGGGTGTGTGC
GTGGGGGGGTGAGCAGGGGGTGTGGGCGCGTCGGTCGGGCTGCAACCCCCCCTGCACCCC
CCTCCCCGAGTTGCTGAGCACGGCCCGGCTTCGGGTGCGGGGCTCCGTACGGGGCGTGGC
GCGGGGCTCGCCGTGCCGGGGGGGGGGTGGCGGCAGGTGGGGGTGCCGGGCGGGGGGGGG
CCGCCTCGGGCCGGGGAGGGCTCGGGGGAGGGGCGCGGCGGCCCCCGGAGCGCCGGCGGC
TGTCGAGGCGCGGCGAGCCGCAGCCATTGCCTTTTATGGTAATCGTGCGAGAGGGCGCAG
GGACTTCCTTTGTCCCAAATCTGTGCGGAGCCGAAATCTGGGAGGCGCCGCCGCACCCCC
TCTAGCGGGCGCGGGGCGAAGCGGTGCGGCGCCGGCAGGAAGGAAATGGGGGGGGAGGGC
CTTCGTGCGTCGCCGCGCCGCCGTCCCCTTCTCCCTCTCCAGCCTCGGGGCTGTCCGCGG
GGGGACGGCTGCCTTCGGGGGGGACGGGGCAGGGGGGGGTTCGGCTTCTGGCGTGTGACC
GGCGGCTCTAGAGCCTCTGCTAACCATGTTCATGCCTTCTTCTTTTTCCTACAGCTCCTG
GGCAACGTGCTGGTTATTGTGCTGTCTCATCATTTTGGCAAAGAATTCGATATCAAGCTT
GGTACCGAGCTCGGATCCACTAGTAACGGCCGCCAGTGTGCTGGAATTCGCCCTTCCGCA
GCGCCAGCATGCACAGCTCAGCACTGCTCTGTTGCCTGGTCCTCCTGACTGGGGTGAGGG
CCAGCCCAGGCCAGGGCACCCAGTCTGAGAACAGCTGCACCCACTTCCCAGGCAACCTGC
CTAACATGCTTCGAGATCTCCGAGATGCCTTCAGCAGAGTGAAGACTTTCTTTCAAATGA
AGGATCAGCTGGACAACTTGTTGTTAAAGGAGTCCTTGCTGGAGGACTTTAAGGGTTACC
TGGGTTGCCAAGCCTTGTCTGAGATGATCCAGTTTTACCTGGAGGAGGTGATGCCCCAAG
CTGAGAACCAAGACCCAGACATCAAGGCGCATGTGAACTCCCTGGGGGAGAACCTGAAGA
CCCTCAGGCTGAGGCTACGGCGCTGTCATCGATCTCTTCCCTGTGAAAACAAGAGCAAGG
CCGTGGAGCAGGTGAAGAATGCCTTTAATAAGCTCCAAGAGAAAGGCATCTACAAAGCCA
TGAGTGAGTTTGACATCTTCATCAACTACATAGAAGCCTACATGACAATGAAGATACGAA
ACTGAGACATCAGGGTGGCAAGCCGAATTCCCCCTAGCGGAAGGGCGAATTCTGCAGAAT
CGGATCCATCGATACCGTCGACCTCGAGGGGGGGCCCGGTACCCAATTCGCCCTATAGTG
AGTCGTATTACGCGCGCAGCGGCCGACCATGGCCCAACTTGTTTATTGCAGCTTATAATG
GTTACAAATAAAGCAATAGCATCACAAATTTCACAAATAAAGCATTTTTTTCACTGCATT
CTAGTTGTGGTTTGTCCAAACTCATCAATGTATCTTATCATGTCTGGATCTCCGGACACG
TGCGGACCGAGCGGCCGCAGGAACCCCTAGTGATGGAGTTGGCCACTCCCTCTCTGCGCG
CTCGCTCGCTCACTGAGGCCGGGCGACCAAAGGTCGCCCGACGCCCGGGCTTTGCCCGGG
CGGCCTCAGTGAGCGAGCGAGCGCGCAGCTGCCTGCAGGTCTGAGACAATAACCCTGATA
AATGCTTCAATAATGTA
Human IL-10:
(SEQ ID NO: 3)
MHSSALLCCLVLLTGVRASPGQGTQSENSCTHFPGNLPNMLRDLRDAFSRVKTFFQMKDQ
LDNLLLKESLLEDFKGYLGCQALSEMIQFYLEEVMPQAENQDPDIKAHVNSLGENLKTLR
LRLRRCHRFLPCENKSKAVEQVKNAFNKLQEKGIYKAMSEFDIFINYIEAYMTMKIRN
Rat IL-10:
(SEQ ID NO: 4)
MPGSALLCCLLLLAGVKTSKGHSIRGDNNCTHFPVSQTHMLRELRAAFSQVKTFFQKKDQ
LDNILLTDSLLQDFKGYLGCQALSEMIKFYLVEVMPQAENHGPEIKEHLNSLGEKLKTLW
IQLRRCHRFLPCENKSKAVEQVKNDENKLQDKGVYKAMNEFDIFINCIEAYVTLKMKN
Rat IL-10:
(SEQ ID NO: 5)
MLGSALLCCLLLLAGVKTSKGHSIRGDNNCTHFPVSQTHMLRELRAAFSQVKTFFQKKDQ
LDNIVLTDSLLQDFKGYLGCQALSEMIKFYLVEVMPQAENHGPEIKEHLNSLGEKLKTLW
IQLRRCHRFLPCENKSKAVEQVKNDFNKLQDKGVYKAMNEFDIFINCIEAYVTLKMKN
Mouse IL-10:
(SEQ ID NO: 6)
MPGSALLCCLLLLTGMRISRGQYSREDNNCTHFPVGQSHMLLELRTAFSQVKTFFQTKDQ
LDNILLTDSLMQDFKGYLGCQALSEMIQFYLVEVMPQAEKHGPEIKEHLNSLGEKLKTLR
MRLRRCHRFLPCENKSKAVEQVKSDFNKLQDQGVYKAMNEFDIFINCIEAYMMIKMKS
Horse IL-10:
(SEQ ID NO: 7)
MHSSALLCYLVFLAGVGASRDRGTQSENSCTHFPTSLPHMLHELRAAFSRVKTFFQMKDQ
LDNMLLNGSLLEDFKGYLGCQALSEMIQFYLEEVMPQAENHGPDIKEHVNSLGEKLKTLR
VRLRRCHRFLPCENKSKAVEQVKSAFSKLQEKGVYKAMSEFDIFINYIEAYMTTKMKN
Cat IL-10:
(SEQ ID NO: 8)
MHSSALLCFLVFLAGVGASRHQSTLSEDNCTHESVSLPHMLRELRAAFGKVKTFFQTKDE
LHSILLTRSLLEDFKGYLGCQALSEMIQFYLEEVMPQAENEDPDIKQHVNSLGEKLKTLR
LRLRRCHRFLPCENKSKVVEQVKSTFSKLQEKGVYKAMGEFDIFINYIEAYMTMKMKI
Guinea pig IL-10:
(SEQ ID NO: 9)
MPGSALLCCLALLAGVKASQGTNTQSEDSCAHFPAGLPHMLRELRAAFGRVKTFFQTQDQ
LDNVLLNKSLLEDFKGYLGCQALSEMIQFYLVEVMPQAEKHGPEIKEHLNSLGEKLKTLR
MRLRRCHRFLPCENKSKAVEQVKSDFNKLQDQGVYKAMNEFDIFINCIEAYMMIKMKS
Llama IL-10:
(SEQ ID NO: 10)
MPRSALLCCLILLAGVAASRDQGTQSENSCAHFPASLPHMLRELRAAFGRVKTFFQMKDQ
LDNMLLTRSLLEDFKGYLGCQALSEMIQFYLEEVMPQAENHGPDIKEHVNSLGEKLKTLR
LRLRRCHRFLPCENKSKAVEQVRGVFSKLQEKGVYKAMSEFDIFINYIEAYMTMKMKN
Gerbil IL-10:
(SEQ ID NO: 11)
MPSSALLYCLILLAGVRPSRGEYPRNESNCTHFPVSQTHMLRELRAAFSQVKTFFQKKDQ
LDNILLTDSLQQDFKGYLGCQALSEMIQFYLVEVMPQAENHGPEIKENLNSLGEKLKTLR
MQLRRCHRFLPCENKSKAVEQVKNDFNKLQEKGVYKAMNEFDIFINCIEAYMTIKMKS
Chimp IL-10:
(SEQ ID NO: 12)
MHSSALLCCLVLLTGVRASPGQGTQSENSCTHFPGNLPNMLRDLRDAFSRVKTFFQMKDQ
LDNLLLKESLLEDFKGYLGCQALXEMIQFYLEEVMPQAENQDPDIKVHVNSLGENLKTLR
LRLRRCHRFLPCENKSKAVEQVKNAFNKLQEKGIYKAMSEFDIFINYIEAYMTMKIRN
Rhesus macaques IL-10:
(SEQ ID NO: 13)
MHSSALLCCLVLLTGVRASPGQGTQSENSCTRFPGNLPHMLRDLRDAFSRVKTFFQMKDQ
LDNILLKESLLEDFKGYLGCQALSEMIQFYLEEVMPQAENHDPDIKEHVNSLGENLKTLR
LRLRRCHRFLPCENKSKAVEQVKNAFSKLQEKGVYKAMSEFDIFINYIEAYMTMKIQN
Marmoset IL-10:
(SEQ ID NO: 14)
MHSSALLCCLVFLTGVRASPGQGTQSENSCTHFPGSLPHMLRELRVAFSRVKTFFQKKDQ
LDSMLLKESLLEDFKGYLGCQALSEMIQFYLEEVMPQAENHDPDIKEHVNSLGEKLKTFR
LRLRRCHRFLPCENKSKAVVQVKNAVSKLQEKGIYKAMSEFDIFIDYIEAYMTMKAQN
Pig-tailed macaque IL-10:
(SEQ ID NO: 15)
MHSSALLCCLVLLTGVRASPGQGTQSENSCTRFPGNLPHMLRDLRDAFSRVKTFFQMKDQ
LDNILLKESLLEDFKGYLGCQALSEMIQFYLEEVMPQAENHDPDIKEHVNSLGENLKTLR
LRLRRCHRFLPCENKSKAVEQVMNAFSKLQEKGVYKAMSEFDIFINYIEAYMTMKIQN
Cynomolgus monkey IL-10:
(SEQ ID NO: 16)
MHSSALLCCLVLLTGVRASPGQGTQSENSCTRFPGNLPHMLRDLRDAFSRVKTFFQMKDQ
LDNILLKESLLEDFKGYLGCQALSEMIQFYLEEVMPQAENHDPDIKEHVNSLGENLKTLR
LRLRRCHRFLPCENKSKAVEQVKNAFSKLQEKGVYKAMSEFDIFINYIEAYMTMKIRN
Sooty mangabey IL-10:
(SEQ ID NO: 17)
MHSSALLCCLVLLTGVRASPGQGTQSENSCTRFPGNLPHMLRDLRDVFSRVKTFFQMKDQ
LDNILLKESLLEDFKGYLGCQALSEMIQFYLEEVMPQAENHDPDIKEHVNSLGENLKTLR
LRLRRCHRFLPCENKSKAVEQVKNAFSKLQEKGVYKAMSEFDIFINYIEAYMTMKIQN
Squirrel monkey IL-10:
(SEQ ID NO: 18)
MHSSALLCCLVFLTGVRASPGQGTQSENSCTHFPGSLPHMLRELRVAFGRVKTFFQKKDQ
LDSMLLKESLLEDFKGYLGCQALSEMIQFYLEEVMPQAENHDPDIKEHVNSLGEKLKTFR
LRLRRCHRFLPCENKSKAVAQVKNAVSKLQEKGVYKAMSEFDIFIDYIEAYMTMKTQN
Goat IL-10:
(SEQ ID NO: 19)
MPSSSALLCCLVFLAGVAASRDASTLSDSSCTHFPASLPHMLRELRAAFGKVKTFFQMKD
QLNSMLLTQSLLDDFKGYLGCQALSEMIQFYLEEVMPQAENHGPDIKEHVNSLGEKLKTL
RLRLRRCHRFLPCENKSKAVEQVKRVFNMLQERGVYKAMSEFDIFINYIESYMTTKM
Sheep IL-10:
(SEQ ID NO: 20)
MPSSSAVLCCLVFLAGVAASRDASTLSDSSCTHFPASLPHMLRDVRAAFGKVKTFFQMKD
QLNSMLLTQSLLDDFKGYLGCQALSEMIQFYLEEVMPQAENHGPDIKEHVNSLGEKLKTL
RLRLRRCHRFLPCENKSKAVEQVKRVFNMLQERGVYKAMSEFDIFINYIESYMTTKM
Dog IL-10:
(SEQ ID NO: 21)
MHGSALLCCCLVLLAGVGASRHQSTLLEDDCTHFPASLPHMLRELRAAFGRVKIFFQMKD
KLDNILLTGSLLEDFKSYLGCQALSEMIQFYLEEVMPRAENHDPDIKNHVNSLGEKLKTL
RLRLRLRRCHRFLPCENKSKAVEQVKSAFSKLQEKGVYKAMSEFDIFINYIETYMTMRMK
I
Ferret IL-10:
(SEQ ID NO: 22)
MNGEASPHPHIKGGPVGEGLHTRGLLLQHQTTRPALGRQSFTMPSPALLCCLVLLAGVGA
SRHQSALSEDNCTHFPASLPHMLRELRAAFGRVKTFFQMKDKLGSILLTGSLLEDFKGYL
GCQALSEMIQFYLEEVMPQAENHDPEVKEHVNSLGEKLKTLRLRLRRCHRFLPCENKSKA
VEQVKSAFSKLQERGVYKAMSEFDIFINYIETYMTMRMKI
Rabbit IL-10:
(SEQ ID NO: 23)
MLSSALLCCLVFLGGTGASRGQDTPAENSCIHFPGGLPHMLRELRAAFGRVKTFFQSKDQ
LNSMLLTESLLEDLKGYLGCQALSEMIQFYLKDVMPQAENHSPAIREHVNSLGENLKTLR
LRLRQCHRFLPCENKSKAVEQVKSAFSKLQEEGVYKAMSEFDIFINYIETYMTMKIKS
Golden hamster IL-10:
(SEQ ID NO: 24)
MLGSALLCCLLLLAGVGPSRGQYTQHESNCTHFPVSQTHMLRELRTAFSQVKTFFQKKDQ
LDNILLTDSLLQDFKGYLGCQTLSEMIQFYLVEVMPQAENHGPEIKEHLNSLGEKLKTLR
RQLQRCHRFLPCENKSKAVEQVKDNENKLQEKGVFKAMNEFDIFINCIEVYMTIKMKS
Chinese Hamster IL-10:
(SEQ ID NO: 25)
MPVSALLCCLLLLAGVGPSRGQYTQQENNCTHFPVSQTHMLRELRTAFSQVKTFFQKKDQ
LDNILLTDSLVQDFEGYLGCQTLSEMIQFYLVEVMPQAENHGPEIKEHLNSLGEKLKTLR
RQLQRCHRFLPCENKSKAVEKVKSDFNKLQEKGVYKAMNEFDIFINCIETYMTIKMKS
Human IL-10F129S:
(SEQ ID NO: 27)
ATGCACAGCTCAGCACTGCTCTGTTGCCTGGTCCTCCTGACTGGGGTGAGGGCCAGCCCA
GGCCAGGGCACCCAGTCTGAGAACAGCTGCACCCACTTCCCAGGCAACCTGCCTAACATG
CTTCGAGATCTCCGAGATGCCTTCAGCAGAGTGAAGACTTTCTTTCAAATGAAGGATCAG
CTGGACAACTTGTTGTTAAAGGAGTCCTTGCTGGAGGACTTTAAGGGTTACCTGGGTTGC
CAAGCCTTGTCTGAGATGATCCAGTTTTACCTGGAGGAGGTGATGCCCCAAGCTGAGAAC
CAAGACCCAGACATCAAGGCGCATGTGAACTCCCTGGGGGAGAACCTGAAGACCCTCAGG
CTGAGGCTACGGCGCTGTCATCGATCTCTTCCCTGTGAAAACAAGAGCAAGGCCGTGGAG
CAGGTGAAGAATGCCTTTAATAAGCTCCAAGAGAAAGGCATCTACAAAGCCATGAGTGAG
TTTGACATCTTCATCAACTACATAGAAGCCTACATGACAATGAAGATACGAAACTGAG
Human IL-10:
(SEQ ID NO: 28)
ATGCACAGCTCAGCACTGCTCTGTTGCCTGGTCCTCCTGACTGGGGTGAGGGCCAGCCCA
GGCCAGGGCACCCAGTCTGAGAACAGCTGCACCCACTTCCCAGGCAACCTGCCTAACATG
CTTCGAGATCTCCGAGATGCCTTCAGCAGAGTGAAGACTTTCTTTCAAATGAAGGATCAG
CTGGACAACTTGTTGTTAAAGGAGTCCTTGCTGGAGGACTTTAAGGGTTACCTGGGTTGC
CAAGCCTTGTCTGAGATGATCCAGTTTTACCTGGAGGAGGTGATGCCCCAAGCTGAGAAC
CAAGACCCAGACATCAAGGCGCATGTGAACTCCCTGGGGGAGAACCTGAAGACCCTCAGG
CTGAGGCTACGGCGCTGTCATCGATTTCTTCCCTGTGAAAACAAGAGCAAGGCCGTGGAG
CAGGTGAAGAATGCCTTTAATAAGCTCCAAGAGAAAGGCATCTACAAAGCCATGAGTGAG
TTTGACATCTTCATCAACTACATAGAAGCCTACATGACAATGAAGATACGAAACTGAG
XT-151 Plasmid:
(SEQ ID NO: 29)
AGCTTTGCAGCCGGGCGTAATAGCGAAGAGGCCCGCACCGATCGCCCTTCCCAACAGTTG
CGCAGCCTGAATGGCGAATGGACGCGCCCTGTAGCGGCGCATTAAGCGCGGCGGGTGTGG
TGGTTACGCGCAGCGTGACCGCTACACTTGCCAGCGCCCTAGCGCCCGCTCCTTTCGCTT
TCTTCCCTTCCTTTCTCGCCACGTTCGCCGGCTTTCCCCGTCAAGCTCTAAATCGGGGGC
TCCCTTTAGGGTTCCGATTTAGTGCTTTACGGCACCTCGACCCCAAAAAACTTGATTAGG
GTGATGGTTCACGTAGTGGGCCATCGCCCTGATAGACGGTTTTTCGCCCTTTGACGTTGG
AGTCCACGTTCTTTAATAGTGGACTCTTGTTCCAAACTGGAACAACACTCAACCCTATCT
CGGTCTATTCTTTTGATTTATAAGGGATTTTGCCGATTTCGGCCTATTGGTTAAAAAATG
AGCTGATTTAACAAAAATTTAACGCGAATTTTAACAAAATATTAACGCTTACAATTTCCT
GATGCGGTATTTTCTCCTTACGCATCTGTGCGGTATTTCACACCGCATATGGTGCACTCT
CAGTACAATCTGCTCTGATGCCGCATAGTTAAGCCAGCCCCGACACCCGCCAACACCCGC
TGACGCGCCCTGACGGGCTTGTCTGCTCCCGGCATCCGCTTACAGACAAGCTGTGACCGT
CTCCGGGAGCTGCATGTGTCAGAGGTTTTCACCGTCATCACCGAAACGCGCGAGACGAAA
GGGCCTCGTGATACGCCTATTTTTATAGGTTAATGTCATGATAATAATGGTTTCTTAGAC
GTCAGGTGGCACTTTTCGGGGAAATGTGCGCGGAACCCCTATTTGTTTATTTTTCTAAAT
ACATTCAAATATGTATCCGCTCATGAGACAATAACCCTGATAAATGCTTCAATAATATTG
AAAAAGGAAGAGTAATTGAAACAAGATGGATTGCACGCAGGTTCTCCGGCCGCTTGGGTG
GAGAGGCTATTCGGCTATGACTGGGCACAACAGACAATCGGCTGCTCTGATGCCGCCGTG
TTCCGGCTGTCAGCGCAGGGGCGCCCGGTTCTTTTTGTCAAGACCGACCTGTCCGGTGCC
CTGAATGAACTGCAGGACGAGGCAGCGCGGCTATCGTGGCTGGCCACGACGGGCGTTCCT
TGCGCAGCTGTGCTCGACGTTGTCACTGAAGCGGGAAGGGACTGGCTGCTATTGGGCGAA
GTGCCGGGGCAGGATCTCCTGTCATCTCACCTTGCTCCTGCCGAGAAAGTATCCATCATG
GCTGATGCAATGCGGCGGCTGCATACGCTTGATCCGGCTACCTGCCCATTCGACCACCAA
GCGAAACATCGCATCGAGCGAGCACGTACTCGGATGGAAGCCGGTCTTGTCGATCAGGAT
GATCTGGACGAAGAGCATCAGGGGCTCGCGCCAGCCGAACTGTTCGCCAGGCTCAAGGCG
CGCATGCCCGACGGCGAGGATCTCGTCGTGACCCATGGCGATGCCTGCTTGCCGAATATC
ATGGTGGAAAATGGCCGCTTTTCTGGATTCATCGACTGTGGCCGGCTGGGTGTGGCGGAC
CGCTATCAGGACATAGCGTTGGCTACCCGTGATATTGCTGAAGAGCTTGGCGGCGAATGG
GCTGACCGCTTCCTCGTGCTTTACGGTATCGCCGCTCCCGATTCGCAGCGCATCGCCTTC
TATCGCCTTCTTGACGAGTTCTTCTGAGTAACCGTCAGACCAAGTTTACTCATATATACT
TTAGATTGATTTAAAACTTCATTTTTAATTTAAAAGGATCTAGGTGAAGATCCTTTTTGA
TAATCTCATGACCAAAATCCCTTAACGTGAGTTTTCGTTCCACTGAGCGTCAGACCCCGT
AGAAAAGATCAAAGGATCTTCTTGAGATCCTTTTTTTCTGCGCGTAATCTGCTGCTTGCA
AACAAAAAAACCACCGCTACCAGCGGTGGTTTGTTTGCCGGATCAAGAGCTACCAACTCT
TTTTCCGAAGGTAACTGGCTTCAGCAGAGCGCAGATACCAAATACTGTTCTTCTAGTGTA
GCCGTAGTTAGGCCACCACTTCAAGAACTCTGTAGCACCGCCTACATACCTCGCTCTGCT
AATCCTGTTACCAGTGGCTGCTGCCAGTGGCGATAAGTCGTGTCTTACCGGGTTGGACTC
AAGACGATAGTTACCGGATAAGGCGCAGCGGTCGGGCTGAACGGGGGGTTCGTGCACACA
GCCCAGCTTGGAGCGAACGACCTACACCGAACTGAGATACCTACAGCGTGAGCTATGAGA
AAGCGCCACGCTTCCCGAAGGGAGAAAGGCGGACAGGTATCCGGTAAGCGGCAGGGTCGG
AACAGGAGAGCGCACGAGGGAGCTTCCAGGGGGAAACGCCTGGTATCTTTATAGTCCTGT
CGGGTTTCGCCACCTCTGACTTGAGCGTCGATTTTTGTGATGCTCGTCAGGGGGGCGGAG
CCTATGGAAAAACGCCAGCAACGCGGCCTTTTTACGGTTCCTGGCCTTTTGCTGGCCTTT
TGCTCACATGTTCTTTCCTGCGTTATCCCCTGATTCTGTGGATAACCGTATTACCGCCTT
TGAGTGAGCTGATACCGCTCGCCGCAGCCGAACGACCGAGCGCAGCGAGTCAGTGAGCGA
GGAAGCGGAAGAGCGCCCAATACGCAAACCGCCTCTCCCCGCGCGTTGGCCGATTCATTA
ATGCAGGGCTGCAGCATGCCTGCAGGCAGCTGCGCGCTCGCTCGCTCACTGAGGCCGCCC
GGGCGTCGGGCGACCTTTGGTCGCCCGGCCTCAGTGAGCGAGCGAGCGCGCAGAGAGGGA
GTGGCCAACTCCATCACTAGGGGTTCCTGCGGCCGCACGCGTGCTCCGGTGCCCGTCAGT
GGGCAGAGCGCACATCGCCCACAGTCCCCGAGAAGTTGGGGGGAGGGGTCGGCAATTGAA
CCGGTGCCTAGAGAAGGTGGCGCGGGGTAAACTGGGAAAGTGATGTCGTGTACTGGCTCC
GCCTTTTTCCCGAGGGTGGGGGAGAACCGTATATAAGTGCAGTAGTCGCCGTGAACGTTC
TTTTTCGCAACGGGTTTGCCGCCAGAACACAGCTGGTGGGGTAGGGATGAGGGAGGGAGG
GGCATTGTGATGTACAGGGCTGCTCTGTGAGATCAAGGGTCTCTTAAGGGTGGGAGCTGG
GGCAGGGACTACGAGAGCAGCCAGATGGGCTGAAAGTGGAACTCAAGGGGTTTCTGGCAC
CTACCTACCTGCTTCCCGCTGGGGGGTGGGGAGTTGGCCCAGAGTCTTAAGATTGGGGCA
GGGTGGAGAGGTGGGCTCTTCCTGCTTCCCACTCATCTTATAGCTTTCTTTCCCCAGATC
CGAATTCGAGATCCAAACCAAGGAGGAAAGGATATCACAGAGGAGACCTCGAGCCCATCA
ACAAGTTTGTACAAAAAAGCAGGCACCATGCTGCCGTGCCTCGTAGTGCTGCTGGCGGCG
CTCCTCAGCCTCCGTCTTGGCTCAGACGCTCATGGGACAGAGCTGCCCAGCCCTCCGTCT
GTGTGGTTTGAAGCAGAATTTTTCCACCACATCCTCCACTGGACACCCATCCCAAATCAG
TCTGAAAGTACCTGCTATGAAGTGGCGCTCCTGAGGTATGGAATAGAGTCCTGGAACTCC
ATCTCCAACTGTAGCCAGACCCTGTCCTATGACCTTACCGCAGTGACCTTGGACCTGTAC
CACAGCAATGGCTACCGGGCCAGAGTGCGGGCTGTGGACGGCAGCCGGCACTCCAACTGG
ACCGTCACCAACACCCGCTTCTCTGTGGATGAAGTGACTCTGACAGTTGGCAGTGTGAAC
CTAGAGATCCACAATGGCTTCATCCTCGGGAAGATTCAGCTACCCAGGCCCAAGATGGCC
CCCGCAAATGACACATATGAAAGCATCTTCAGTCACTTCCGAGAGTATGAGATTGCCATT
CGCAAGGTGCCGGGAAACTTCACGTTCACACACAAGAAAGTAAAACATGAAAACTTCAGC
CTCCTAACCTCTGGAGAAGTGGGAGAGTTCTGTGTCCAGGTGAAACCATCTGTCGCTTCC
CGAAGTAACAAGGGGATGTGGTCTAAAGAGGAGTGCATCTCCCTCACCAGGCAGTATTTC
ACCGTGACCAACGTCATCATCTTCTTTGCCTTTGTCCTGCTGCTCTCCGGAGCCCTCGCC
TACTGCCTGGCCCTCCAGCTGTATGTGCGGCGCCGAAAGAAGCTACCCAGTGTCCTGCTC
TTCAAGAAGCCCAGCCCCTTCATCTTCATCAGCCAGCGTCCCTCCCCAGAGACCCAAGAC
ACCATCCACCCGCTTGATGAGGAGGCCTTTTTGAAGGTGTCCCCAGAGCTGAAGAACTTG
GACCTGCACGGCAGCACAGACAGTGGCTTTGGCAGCACCAAGCCATCCCTGCAGACTGAA
GAGCCCCAGTTCCTCCTCCCTGACCCTCACCCCCAGGCTGACAGAACGCTGGGAAACGGG
GAGCCCCCTGTGCTGGGGGACAGCTGCAGTAGTGGCAGCAGCAATAGCACAGACAGCGGG
ATCTGCCTGCAGGAGCCCAGCCTGAGCCCCAGCACAGGGCCCACCTGGGAGCAACAGGTG
GGGAGCAACAGCAGGGGCCAGGATGACAGTGGCATTGACTTAGTTCAAAACTCTGAGGGC
CGGGCTGGGGACACACAGGGTGGCTCGGCCTTGGGCCACCACAGTCCCCCGGAGCCTGAG
GTGCCTGGGGAAGAAGACCCAGCTGCTGTGGCATTCCAGGGTTACCTGAGGCAGACCAGA
TGTGCTGAAGAGAAGGCAACCAAGACAGGCTGCCTGGAGGAAGAATCGCCCTTGACAGAT
GGCCTTGGCCCCAAATTCGGGAGATGCCTGGTTGATGAGGCAGGCTTGCATCCACCAGCC
CTGGCCAAGGGCTATTTGAAACAGGATCCTCTAGAAATGACTCTGGCTTCCTCAGGGGCC
CCAACGGGACAGTGGAACCAGCCCACTGAGGAATGGTCACTCCTGGCCTTGAGCAGCTGC
AGTGACCTGGGAATATCTGACTGGAGCTTTGCCCATGACCTTGCCCCTCTAGGCTGTGTG
GCAGCCCCAGGTGGTCTCCTGGGCAGCTTTAACTCAGACCTGGTCACCCTGCCCCTCATC
TCTAGCCTGCAGTCAAGTGAGCGGGCCAAGCGGCAGCTGCTGAACTTCGACCTGCTGAAG
CTGGCCGGCGACGTGGAGAGCAACCCCGGGCCCATGCACAGCTCAGCACTGCTCTGTTGC
CTGGTCCTCCTGACTGGGGTGAGGGCCAGCCCAGGCCAGGGCACCCAGTCTGAGAACAGC
TGCACCCACTTCCCAGGCAACCTGCCTAACATGCTTCGAGATCTCCGAGATGCCTTCAGC
AGAGTGAAGACTTTCTTTCAAATGAAGGATCAGCTGGACAACTTGTTGTTAAAGGAGTCC
TTGCTGGAGGACTTTAAGGGTTACCTGGGTTGCCAAGCCTTGTCTGAGATGATCCAGTTT
TACCTGGAGGAGGTGATGCCCCAAGCTGAGAACCAAGACCCAGACATCAAGGCGCATGTG
AACTCCCTGGGGGAGAACCTGAAGACCCTCAGGCTGAGGCTACGGCGCTGTCATCGATCT
CTTCCCTGTGAAAACAAGAGCAAGGCCGTGGAGCAGGTGAAGAATGCCTTTAATAAGCTC
CAAGAGAAAGGCATCTACAAAGCCATGAGTGAGTTTGACATCTTCATCAACTACATAGAA
GCCTACATGACAATGAAGATACGAAACTGAACCCAGCTTTCTTGTACAAAGTGGTGCTAG
CACTCTCAGTACAATCTGCTCTGATGCCGCATAGTTAAGCCAGTATCTGCTCCCTGCTTG
TGTGTTGGAGGTCGCTGAGTAGTGCGCGAGCAAAATTTAAGCTACAACAAGGCAAGGCTT
GACCGACAATTGCATGAAGAATCTGCTTAGGGTTAGGCGTTTTGCGCTGCTTCGCGATGT
ACGGGCCAGATATACGCGTACCGGTTAGTAATGATCGACAATCAACCTCTGGATTACAAA
ATTTGTGAAAGATTGACTGGTATTCTTAACTATGTTGCTCCTTTTACGCTATGTGGATAC
GCTGCTTTAATGCCTTTGTATCATGCTATTGCTTCCCGTATGGCTTTCATTTTCTCCTCC
TTGTATAAATCCTGGTTGCTGTCTCTTTATGAGGAGTTGTGGCCCGTTGTCAGGCAACGT
GGCGTGGTGTGCACTGTGTTTGCTGACGCAACCCCCACTGGTTGGGGCATTGCCACCACC
TGTCAGCTCCTTTCCGGGACTTTCGCTTTCCCCCTCCCTATTGCCACGGCGGAACTCATC
GCCGCCTGCCTTGCCCGCTGCTGGACAGGGGCTCGGCTGTTGGGCACTGACAATTCCGTG
GTGTTGTCGGGGAAATCATCGTCCTTTCCTTGGCTGCTCGCCTATGTTGCCACCTGGATT
CTGCGCGGGACGTCCTTCTGCTACGTCCCTTCGGCCCTCAATCCAGCGGACCTTCCTTCC
CGCGGCCTGCTGCCGGCTCTGCGGCCTCTTCCGCGTCTTCGCCTTCGCCCTCAGACGAGT
CGGATCTCCCTTTGGGCCGCCTCCCCGCCTGGCGATGGTACCCAATTCGCCCTATAGTGA
GTCGTATTACGCGCGCAGCGGCCGACCATGGCCCAACTTGTTTATTGCAGCTTATAATGG
TTACAAATAAAGCAATAGCATCACAAATTTCACAAATAAAGCATTTTTTTCACTGCATTC
TAGTTGTGGTTTGTCCAAACTCATCAATGTATCTTATCATGTCTGGATCTCCGGACACGT
GCGGACCGAGCGGCCGCAGGAACCCCTAGTGATGGAGTTGGCCACTCCCTCTCTGCGCGC
TCGCTCGCTCACTGAGGCCGGGCGACCAAAGGTCGCCCGACGCCCGGGCTTTGCCCGGGC
GGCCTCAGTGAGCGAGCGAGCGCGCAGCTGCCTGCAGGTCTGAGACAATAACCCTGATAA
ATGCTTCAATAATGTA

### Compositions

The present disclosure also provides various pharmaceutical compositions. For additional details on the compounds of Formula (I) and DNA (e.g., double-stranded DNA (e.g., plasmid DNA)) used in the compositions, see the above subsections under “Detailed Description of the Invention” entitled “Compounds” and “DNA”.

The present disclosure provides pharmaceutical compositions comprising a compound of Formula (I), or a pharmaceutically acceptable salt thereof, and optionally a pharmaceutically acceptable excipient. In certain embodiments, the pharmaceutical composition described herein comprises a compound of Formula (I), or a pharmaceutically acceptable salt thereof, and a pharmaceutically acceptable excipient.

In some embodiments, the present disclosure provides compositions comprising a compound of Formula (I) and DNA. In some embodiments, the composition further comprises a pharmaceutically acceptable excipient.

In some embodiments, the present disclosure provides compositions comprising a compound of Formula (I) and double-stranded DNA. In some embodiments, the composition further comprises a pharmaceutically acceptable excipient.

In certain embodiments, the present disclosure provides compositions comprising a compound of Formula (I) and a plasmid. In some embodiments, the composition further comprises a pharmaceutically acceptable excipient. In some embodiments, the plasmid encodes a transgene. In some embodiments, the plasmid encodes a cytokine, peptide hormone, glycoprotein hormone, or antibody. In some embodiments, the plasmid encodes a cytokine. In some embodiments, the cytokine is IL-10, IL-36RN, IL-33, or IL-37. In certain embodiments, the plasmid encodes a glycoprotein cytokine. In some embodiments, the plasmid encodes a erythropoietin. In certain embodiments, the plasmid encodes a glycoprotein hormone. In some embodiments, the plasmid encodes an antibody. In some embodiments, the plasmid encodes denosumab or ranibizumab.

In some embodiments, the composition comprises a compound of Formula (I) and a plasmid encoding a IL-10 polypeptide. In some embodiments, the IL-10 polypeptide comprises a mutation. In some embodiments, the mutation is within the portion of the IL-10 polypeptide comprising: LRRCHRFLPCENKSK (SEQ ID NO: 26). In some embodiments, the mutation is within the portion of the IL-10 polypeptide: LRRCHRFLPCENKSK (SEQ ID NO: 26) of any one of SEQ ID NO: 3 through SEQ ID NO: 22, or any other IL-10 peptide. In certain embodiments, the mutation is within the portion of the IL-10 polypeptide; LRRCHRFLPCENKSK (SEQ ID NO: 26) of any one of SEQ ID NO:3 through SEQ ID NO: 22. In some embodiments, the mutation is within the portion of the IL-10 polypeptide; LRRCHRFLPCENKSK (SEQ ID NO: 26) of SEQ ID NO: 3, SEQ ID NO: 7, SEQ ID NO: 8, or SEQ ID NO: 21. In certain embodiments, the mutation is within the portion of the IL-10 polypeptide: LRRCHRFLPCENKSK (SEQ ID NO: 26) of SEQ ID NO: 4 through SEQ ID NO: 6, SEQ ID NO: 9, SEQ ID NO: 11-19, SEQ ID NO: 22-25. In some embodiments, the mutation is a substitution of serine for phenylalanine at: (i) position 129 of any one of SEQ ID NO: 3 through SEQ ID NO: 18 or SEQ ID NO: 23-25; (ii) position 130 of SEQ ID NO: 19 or SEQ ID NO: 20; (iii) position 132 of SEQ ID NO: 21; or (iv) position 171 of SEQ ID NO: 22. In some embodiments, the mutation is a substitution of serine for phenylalanine at position 129 of any one of SEQ ID NO: 3 through SEQ ID NO: 18 or SEQ ID NO: 23-25. In some embodiments, the mutation is a substitution of serine for phenylalanine at position 130 of SEQ ID NO: 19 or SEQ ID NO: 20. In some embodiments, the mutation is a substitution of serine for phenylalanine at position 132 of SEQ ID NO: 21. In some embodiments, the mutation is a substitution of serine for phenylalanine at position 171 of SEQ ID NO: 22. In some embodiments, the plasmid encodes a polypeptide at least 99%, 98%, 95%, 90%, 85%, and 80% identical to any one of SEQ ID NO: 3 through SEQ ID NO: 25. In some embodiments, the plasmid encodes a IL-10 polypeptide at least 99%, 98%, 95%, 90%, 85%, and 80% identical to SEQ ID NO:1. In some embodiments, the plasmid encodes a IL-10 polypeptide comprising the amino acid sequence of SEQ ID NO:1. In certain embodiments, the plasmid encodes a polypeptide at least 99% identical to SEQ ID NO: 1. In some embodiments, the plasmid encodes a polypeptide at least 98% identical to SEQ ID NO: 1. In certain embodiments, the plasmid encodes a polypeptide at least 95% identical to SEQ ID NO: 1. In some embodiments, the plasmid encodes a polypeptide at least 90% identical to SEQ ID NO: 1. In certain embodiments, the plasmid encodes a polypeptide at least 85% identical to SEQ ID NO: 1. In some embodiments, the plasmid encodes a polypeptide at least 80% identical to SEQ ID NO: 1. In certain embodiments, the plasmid comprises the nucleic acid sequence of SEQ ID NO:2. In some embodiments, the plasmid encodes a IL-10 polypeptide at least 99%, 98%, 95%, 90%, 85%, and 80% identical to SEQ ID NO: 2. In some embodiments, the plasmid comprises a nucleic acid at least 99% identical to SEQ ID NO: 2. In certain embodiments, the plasmid comprises a nucleic acid at least 98% identical to SEQ ID NO: 2. In some embodiments, the plasmid comprises a nucleic acid at least 95% identical to SEQ ID NO: 2. In certain embodiments, the plasmid comprises a nucleic acid at least 90% identical to SEQ ID NO: 2. In some embodiments, the plasmid comprises a nucleic acid at least 85% identical to SEQ ID NO: 2. In certain embodiments, the plasmid comprises a nucleic acid at least 80% identical to SEQ ID NO: 2.

In some embodiments, the present disclosure provides compositions comprising a compound of Formula (I) and a DNA minicircle. In some embodiments, the composition further comprises a pharmaceutically acceptable excipient.

In some embodiments, the ratio of DNA:quinacrine ratio is between about 1 to 100. In some embodiments, the ratio of DNA:quinacrine ratio is between about 10-30, 20-40, 30-50, 40-60, 50-70, 60-80, 70-90, or 80-100. In some embodiments, the ratio of DNA:quinacrine ratio is 10-20, 20-30, 30-40, 40-50, 60-70, 80-90, or 90-100. In some embodiments, the ratio of DNA:quinacrine ratio is between about 1, 5, 10, 15, 20, 25, 30, 35, 40, 45, 50, 55, 60, 65, 70, 75, 80, 85, 90, 95, or 100. In some embodiments, the ratio of DNA:quinacrine ratio is between about 1 to 60. In some embodiments, the ratio of DNA:quinacrine ratio is between about 1, 5, 10, 15, 20, 25, 30, 35, 40, 45, 50, 55, or 60. In some embodiments, the ratio of DNA:quinacrine ratio is about 30, 10-20, 30-50, or 20-40.

In some embodiments, the ratio of DNA:a compound of Formula (I) ratio is between about 1 to 100. In some embodiments, the ratio of DNA:a compound of Formula (I) ratio is between about 10-30, 20-40, 30-50, 40-60, 50-70, 60-80, 70-90, or 80-100. In some embodiments, the ratio of DNA:a compound of Formula (I) ratio is 10-20, 20-30, 30-40, 40-50, 60-70, 80-90, or 90-100. In some embodiments, the ratio of DNA:a compound of Formula (I) ratio is between about 1, 5, 10, 15, 20, 25, 30, 35, 40, 45, 50, 55, 60, 65, 70, 75, 80, 85, 90, 95, or 100. In some embodiments, the ratio of DNA:a compound of Formula (I) ratio is between about 1 to 60. In some embodiments, the ratio of DNA:a compound of Formula (I) ratio is between about 1, 5, 10, 15, 20, 25, 30, 35, 40, 45, 50, 55, or 60. In some embodiments, the ratio of DNA:a compound of Formula (I) ratio is about 30, 10-20, 30-50, or 20-40.

In some embodiments, the ratio of pDNA:quinacrine ratio is between about 1 to 100. In some embodiments, the ratio of pDNA:quinacrine ratio is between about 10-30, 20-40, 30-50, 40-60, 50-70, 60-80, 70-90, or 80-100. In some embodiments, the ratio of pDNA:quinacrine ratio is 10-20, 20-30, 30-40, 40-50, 60-70, 80-90, or 90-100. In some embodiments, the ratio of pDNA:quinacrine ratio is between about 1, 5, 10, 15, 20, 25, 30, 35, 40, 45, 50, 55, 60, 65, 70, 75, 80, 85, 90, 95, or 100. In some embodiments, the ratio of pDNA:quinacrine ratio is between about 1 to 60. In some embodiments, the ratio of pDNA:quinacrine ratio is between about 1, 5, 10, 15, 20, 25, 30, 35, 40, 45, 50, 55, or 60. In some embodiments, the ratio of pDNA:quinacrine ratio is about 30, 10-20, 30-50, or 20-40.

In some embodiments, the ratio of pDNA:a compound of Formula (I) ratio is between about 1 to 100. In some embodiments, the ratio of pDNA:a compound of Formula (I) ratio is between about 10-30, 20-40, 30-50, 40-60, 50-70, 60-80, 70-90, or 80-100. In some embodiments, the ratio of pDNA:a compound of Formula (I) ratio is 10-20, 20-30, 30-40, 40-50, 60-70, 80-90, or 90-100. In some embodiments, the ratio of pDNA:a compound of Formula (I) ratio is between about 1, 5, 10, 15, 20, 25, 30, 35, 40, 45, 50, 55, 60, 65, 70, 75, 80, 85, 90, 95, or 100. In some embodiments, the ratio of pDNA:a compound of Formula (I) ratio is between about 1 to 60. In some embodiments, the ratio of pDNA:a compound of Formula (I) ratio is between about 1, 5, 10, 15, 20, 25, 30, 35, 40, 45, 50, 55, or 60. In some embodiments, the ratio of pDNA:a compound of Formula (I) ratio is about 30, 10-20, 30-50, or 20-40.

In certain embodiments, the compound described herein is provided in an effective amount in the pharmaceutical composition. In certain embodiments, the effective amount is a therapeutically effective amount. In certain embodiments, the effective amount is a prophylactically effective amount. In certain embodiments, the effective amount is an amount effective for treating a proliferative disease in a subject in need thereof. In certain embodiments, the effective amount is an amount effective for preventing a proliferative disease in a subject in need thereof. In certain embodiments, the effective amount is an amount effective for treating a hematological disease in a subject in need thereof. In certain embodiments, the effective amount is an amount effective for preventing a hematological disease in a subject in need thereof. In certain embodiments, the effective amount is an amount effective for treating a genetic disease in a subject in need thereof. In certain embodiments, the effective amount is an amount effective for preventing a genetic disease in a subject in need thereof. In certain embodiments, the effective amount is an amount effective for treating a neurological disease in a subject in need thereof. In certain embodiments, the effective amount is an amount effective for preventing a neurological disease in a subject in need thereof. In certain embodiments, the effective amount is an amount effective for treating a in a painful condition subject in need thereof. In certain embodiments, the effective amount is an amount effective for preventing a painful condition in a subject in need thereof. In certain embodiments, the effective amount is an amount effective for treating a psychiatric disorder in a subject in need thereof. In certain embodiments, the effective amount is an amount effective for preventing a psychiatric disorder in a subject in need thereof. In certain embodiments, the effective amount is an amount effective for treating a metabolic disorder in a subject in need thereof. In certain embodiments, the effective amount is an amount effective for preventing a metabolic disorder in a subject in need thereof. In certain embodiments, the effective amount is an amount effective for reducing the risk of developing a disease (e.g., proliferative disease, hematological disease, genetic disease, neurological disease, painful condition, psychiatric disorder, or metabolic disorder) in a subject in need thereof. In certain embodiments, the effective amount is an amount effective for inhibiting the activity (e.g., aberrant activity, such as increased activity) of a protein kinase in a subject or cell.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal, In certain embodiments, the animal is a transgenic animal (e.g., transgenic mice and transgenic pigs). In certain embodiments, the subject is a fish or reptile.

In certain embodiments, the cell is present in vitro. In certain embodiments, the cell is present in vivo.

Pharmaceutical compositions described herein can be prepared by any method known in the art of pharmaceuticals. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses, A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils, Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc,), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof, Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils, Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof, Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol, Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Dosage forms for topical and/or transdermal administration of a compound described herein may include ointments, pastes, creams, lotions, gels, powders, solutions, sprays, inhalants, and/or patches, Generally, the active ingredient is admixed under sterile conditions with a pharmaceutically acceptable carrier or excipient and/or any needed preservatives and/or buffers as can be required, Additionally, the present disclosure contemplates the use of transdermal patches, which often have the added advantage of providing controlled delivery of an active ingredient to the body, Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration, Jet injection devices which deliver liquid formulations to the dermis via a liquid jet injector and/or via a needle which pierces the stratum corneum and produces a jet which reaches the dermis are suitable, Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions, Topically administrable formulations may, for example, comprise from about 1% to about 10% (w/w) active ingredient, although the concentration of the active ingredient can be as high as the solubility limit of the active ingredient in the solvent, Formulations for topical administration may further comprise one or more of the additional ingredients described herein.

A pharmaceutical composition described herein can be prepared, packaged, and/or sold in a formulation for intranasal delivery, Another formulation suitable for intranasal administration is a coarse powder comprising the active ingredient and having an average particle from about 0.2 to 500 micrometers, Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein, A pharmaceutical composition described herein can be prepared, packaged, and/or sold in a formulation for buccal administration, Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein, Alternately, formulations for buccal administration may comprise a powder and/or an aerosolized and/or atomized solution and/or suspension comprising the active ingredient, Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration, Such formulations may, for example, be in the form of eye drops including, for example, a 0.1-1.0% (w/w) solution and/or suspension of the active ingredient in an aqueous or oily liquid carrier or excipient, Such drops may further comprise buffering agents, salts, and/or one or more other of the additional ingredients described herein, Other ophthalmically-administrable formulations which are useful include those which comprise the active ingredient in microcrystalline form and/or in a liposomal preparation, Ear drops and/or eye drops are also contemplated as being within the scope of this disclosure. The formulation may be administered via intravitreal administration and/or subretinal administration.

Although the descriptions of pharmaceutical compositions provided herein are principally directed to pharmaceutical compositions which are suitable for administration to humans, it will be understood by the skilled artisan that such compositions are generally suitable for administration to animals of all sorts, Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder, the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intravitreal, subretinal, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol, Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day.

In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0,001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof, and/or in inhibiting the activity of a protein kinase in a subject or cell), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compound and the additional pharmaceutical agent, but not both. In some embodiments, the additional pharmaceutical agent achieves a desired effect for the same disorder. In some embodiments, the additional pharmaceutical agent achieves different effects.

The compound or composition can be administered concurrently with, prior to, or subsequent to one or more additional pharmaceutical agents, which may be useful as, e.g., combination therapies, Pharmaceutical agents include therapeutically active agents, Pharmaceutical agents also include prophylactically active agents, Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, hematological disease, genetic disease, neurological disease, painful condition, psychiatric disorder, or metabolic disorder), Each additional pharmaceutical agent may be administered at a dose and/or on a time schedule determined for that pharmaceutical agent. The additional pharmaceutical agents may also be administered together with each other and/or with the compound or composition described herein in a single dose or composition or administered separately in different doses or compositions. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, steroidal or non-steroidal anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, anesthetics, anti-coagulants, inhibitors of an enzyme, steroidal agents, steroidal or antihistamine, antigens, vaccines, antibodies, decongestant, sedatives, opioids, analgesics, anti-pyretics, hormones, and prostaglandins. In certain embodiments, the additional pharmaceutical agent is an anti-proliferative agent. In certain embodiments, the additional pharmaceutical agent is an anti-cancer agent. In certain embodiments, the additional pharmaceutical agent is an anti-viral agent. In certain embodiments, the additional pharmaceutical agent is an binder or inhibitor of a protein kinase. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation. In certain embodiments, the compounds described herein or pharmaceutical compositions can be administered in combination with an anti-cancer therapy including, but not limited to, surgery, radiation therapy, transplantation (e.g., stem cell transplantation, bone marrow transplantation), immunotherapy, and chemotherapy, Additional pharmaceutical agents include small organic molecules such as drug compounds (e.g., compounds approved by the US Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins and cells,

### Kits

In one aspect, provided herein are kits comprising: a compound as provided herein or composition provided herein; and instructions for administering to a subject the compound or composition.

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease (e.g., proliferative disease, hematological disease, genetic disease, neurological disease, painful condition, psychiatric disorder, or metabolic disorder) in a subject in need thereof. In certain embodiments, the kits are useful for preventing a disease (e.g., proliferative disease, hematological disease, genetic disease, neurological disease, painful condition, psychiatric disorder, or metabolic disorder) in a subject in need thereof. In certain embodiments, the kits are useful for reducing the risk of developing a disease (e.g., proliferative disease, hematological disease, genetic disease, neurological disease, painful condition, psychiatric disorder, or metabolic disorder) in a subject in need thereof. In certain embodiments, the kits are useful for inhibiting the activity (e.g., aberrant activity, such as increased activity) of a protein kinase in a subject or cell.

In certain embodiments, a kit described herein further includes instructions for using the kit, A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., proliferative disease, hematological disease, genetic disease, neurological disease, painful condition, psychiatric disorder, or metabolic disorder) in a subject in need thereof. In certain embodiments, the kits and instructions provide for preventing a disease (e.g., proliferative disease, hematological disease, genetic disease, neurological disease, painful condition, psychiatric disorder, or metabolic disorder) in a subject in need thereof. In certain embodiments, the kits and instructions provide for reducing the risk of developing a disease (e.g., proliferative disease, hematological disease, genetic disease, neurological disease, painful condition, psychiatric disorder, or metabolic disorder) in a subject in need thereof. In certain embodiments, the kits and instructions provide for inhibiting the activity (e.g., aberrant activity, such as increased activity) of a protein kinase in a subject or cell, A kit described herein may include one or more additional pharmaceutical agents described herein as a separate composition,

### Methods and Uses

Provided herein are methods and uses of delivery, treatment, and prevention.

In some embodiments, provided herein are methods of delivering a compound of Formula (I) to a subject, the method comprising administering an effective amount of a compound disclosed herein or composition as disclosed herein to a subject in need thereof.

In certain embodiments, provided herein are methods of delivering DNA to a subject, the method comprising administering an effective amount of a composition as disclosed herein to a subject in need thereof.

In certain embodiments, provided herein are methods of delivering DNA to a cell of a subject, the method comprising administering an effective amount of a composition as disclosed herein to a subject in need thereof. In some embodiments, the cell is a cell that expresses syndecan, α-dystoglycan CD44, CD44, glypicans, NG2, RPTP-σ, GPI-brevican, RHAMM, Toll-like, IGF-H, mannose, or mannose-6-phosphate receptors. In some embodiments, the cell is a cell that expresses syndecan, CD44, glypicans, NG2, RPTP-σ, GPI-brevican, RHAMM, Toll-like, IGF-II, mannose, or mannose-6-phosphate receptors. In some embodiments, the cell is a cell that expresses IGF-11, mannose, or mannose-6-phosphate receptors.

In certain embodiments, provided herein are methods of delivering double-stranded DNA to a subject, the method comprising administering an effective amount of a composition as disclosed herein to a subject in need thereof.

In certain embodiments, provided herein are methods of delivering double-stranded DNA to a cell of a subject, the method comprising administering an effective amount of a composition as disclosed herein to a subject in need thereof. In some embodiments, the cell is a cell that expresses syndecan, α-dystoglycan CD44, CD44, glypicans, NG2, RPTP-σ, GPI-brevican, RHAMM, Toll-like, IGF-II, mannose, or mannose-6-phosphate receptors. In some embodiments, the cell is a cell that expresses syndecan, CD44, glypicans, NG2, RPTP-σ, GPI-brevican, RHAMM, Toll-like, IGF-II, mannose, or mannose-6-phosphate receptors. In some embodiments, the cell is a cell that expresses IGF-II, mannose, or mannose-6-phosphate receptors.

In some embodiments, provided herein are methods of delivering a plasmid to a subject, the method comprising administering an effective amount of a composition as provided herein to a subject in need thereof.

In some embodiments, provided herein are methods of delivering a plasmid to a cell of a subject, the method comprising administering an effective amount of a composition as provided herein to a subject in need thereof. In some embodiments, the cell is a cell that expresses syndecan, α-dystoglycan CD44, CD44, glypicans, NG2, RPTP-σ, GPI-brevican, RHAMM, Toll-like, IGF-II, mannose, or mannose-6-phosphate receptors. In some embodiments, the cell is a cell that expresses syndecan, CD44, glypicans, NG2, RPTP-σ, GPI-brevican, RHAMM, Toll-like, IGF-II, mannose, or mannose-6-phosphate receptors. In some embodiments, the cell is a cell that expresses IGF-II, mannose, or mannose-6-phosphate receptors.

In some embodiments, provided herein are methods of delivering a DNA minicircle to a subject, the method comprising administering an effective amount of a composition as provided herein to a subject in need thereof.

In some embodiments, provided herein are methods of delivering a DNA minicircle to a cell of a subject, the method comprising administering an effective amount of a composition as provided herein to a subject in need thereof. In some embodiments, the cell is a cell that expresses syndecan, α-dystoglycan CD44, CD44, glypicans, NG2, RPTP-σ, GPI-brevican, RHAMM, Toll-like, IGF-II, mannose, or mannose-6-phosphate receptors. In some embodiments, the cell is a cell that expresses syndecan, CD44, glypicans, NG2, RPTP-σ, GPI-brevican, RHAMM, Toll-like, IGF-II, mannose, or mannose-6-phosphate receptors. In some embodiments, the cell is a cell that expresses IGF-II, mannose, or mannose-6-phosphate receptors.

In certain embodiments, provided herein are methods of treating or preventing a disease or disorder in a subject, the method comprising administering an effective amount of a composition as provided herein to a subject in need thereof. In certain embodiments, provided herein are methods of treating a disease or disorder in a subject, the method comprising administering an effective amount of a composition as provided herein to a subject in need thereof. In certain embodiments, provided herein are methods of preventing a disease or disorder in a subject, the method comprising administering an effective amount of a composition as provided herein to a subject in need thereof. In some embodiments, the disease or disorder is an inflammatory disease, proliferative disease, autoimmune disease, hematological disease, genetic disease, neurological disease, painful condition, metabolic disorder, infectious disease, cardiovascular disease, cerebrovascular disease, tissue repair disorder, pulmonary disease, dermatological disease, bone disease, or hormonal disease. In some embodiments, the disease or disorder is an inflammatory disease. In some embodiments, the disease or disorder is a proliferative disease. In some embodiments, the disease or disorder is an autoimmune disease. In some embodiments, the disease or disorder is a hematological disease. In certain embodiments, the disease or disorder is genetic disease. In some embodiments, the disease or disorder is a neurological disease. In some embodiments, the disease or disorder is a painful condition. In some embodiments, the disease or disorder is a metabolic disorder. In some embodiments, the disease or disorder is an infectious disease. In some embodiments, a plasmid provided herein is used to deliver vaccines. In some embodiments, the disease or disorder is a cardiovascular disease. In some embodiments, the disease or disorder is a cerebrovascular disease. In some embodiments, the disease or disorder is a tissue repair disorder. In some embodiments, the disease or disorder is a pulmonary disease. In some embodiments, the disease or disorder is a dermatological disease. In some embodiments, the disease or disorder is a bone disease. In some embodiments, the disease or disorder is a hormonal disease.

In some embodiments, the disease or disorder is a painful condition, an inflammatory disease, or an autoimmune disease.

In some embodiments, the disease or disorder is a painful condition. In certain embodiments, the painful condition is neuropathic pain. In some embodiments, the painful condition is chronic pain. In some embodiments, the painful condition is due to nerve damage, injury, spinal cord injury, pain associated with spinal disc degeneration, radiculitis, radiculopathy, neuritis, inflammation, thermal or mechanical hyperalgesia, thermal or mechanical allodynia, iatrogenic neuralgia, diabetic pain, pain arising from irritable bowel or other internal organ disorders, endometriosis pain, phantom limb pain, complex regional pain syndromes, fibromyalgia, low back pain, cancer pain, pain arising from infection, inflammation or trauma to peripheral nerves or the central nervous system, multiple sclerosis pain, entrapment pain, pain from HIV infection, herpesvirus infection, and pain from infections. In some embodiments, the painful condition is due to nerve damage. In some embodiments, the painful condition is due to injury. In some embodiments, the painful condition is due to spinal cord injury. In some embodiments, the painful condition is due to pain associated with spinal disc degeneration. In some embodiments, the painful condition is due to radiculitis. In some embodiments, the painful condition is due to radiculopathy. In some embodiments, the painful condition is due to neuritis. In some embodiments, the painful condition is due to inflammation. In some embodiments, the painful condition is due to thermal or mechanical hyperalgesia. In some embodiments, the painful condition is due to thermal or mechanical allodynia. In some embodiments, the painful condition is due to iatrogenic neuralgia. In some embodiments, the painful condition is due to diabetic pain. In some embodiments, the painful condition is due to pain arising from irritable bowel or other internal organ disorders. In some embodiments, the painful condition is due to endometriosis pain. In some embodiments, the painful condition is due to phantom limb pain. In some embodiments, the painful condition is due to complex regional pain syndromes. In some embodiments, the painful condition is due to fibromyalgia. In some embodiments, the painful condition is due to low back pain. In some embodiments, the painful condition is due to cancer pain. In some embodiments, the painful condition is due to pain arising from infection.

In some embodiments, the painful condition is due to inflammation or trauma to peripheral nerves or the central nervous system. In some embodiments, the painful condition is due to multiple sclerosis pain. In some embodiments, the painful condition is due to entrapment pain. In some embodiments, the painful condition is due to pain from HIV infection. In some embodiments, the painful condition is due to herpesvirus infection. In some embodiments, the painful condition is due to pain from infection.

In some embodiments, the inflammation is due to neuroinflammation. In certain embodiments, the neuroinflammation is due to injury, infection, a toxin, or neurodegenerative disease. In certain embodiments, the neuroinflammation is due to injury. In certain embodiments, the neuroinflammation is due to infection. In certain embodiments, the neuroinflammation is due to a toxin. In certain embodiments, the neuroinflammation is due to neurodegenerative disease.

In some embodiments, the disease or disorder is an inflammatory disease. In some embodiments, the inflammatory disease is an inflammatory disease of the bowel, lung, eye, gum, skin, or joints.

In certain embodiments, the inflammatory disease of the bowel is inflammatory bowel disease, Crohn's disease, ileitis, or ulcerative colitis. In certain embodiments, the inflammatory disease of the bowel is inflammatory bowel disease. In certain embodiments, the inflammatory disease of the bowel is Crohn's disease. In certain embodiments, the inflammatory disease of the bowel ileitis. In certain embodiments, the inflammatory disease of the bowel is ulcerative colitis.

In some embodiments, the inflammatory disease of the lung is bronchitis, oxidant-induced lung injury, or chronic obstructive airway disease. In some embodiments, the inflammatory disease of the lung is bronchitis. In some embodiments, the inflammatory disease of the lung is oxidant-induced lung injury. In some embodiments, the inflammatory disease of the lung is chronic obstructive airway disease.

In some embodiments, the inflammatory disease of the eye is corneal dystrophy, ocular hypertension, trachoma, onchocerciasis, retinitis, dry eye, uveitis, sympathetic ophthalmitis and endophthalmitis, optic neuritis, or macular degeneration. In some embodiments, the inflammatory disease of the eye is corneal dystrophy. In some embodiments, the inflammatory disease of the eye is ocular hypertension. In some embodiments, the inflammatory disease of the eye is trachoma. In some embodiments, the inflammatory disease of the eye is onchocerciasis. In some embodiments, the inflammatory disease of the eye is retinitis. In some embodiments, the inflammatory disease of the eye is dry eye. In some embodiments, the inflammatory disease of the eye is uveitis. In some embodiments, the inflammatory disease of the eye is sympathetic ophthalmitis and endophthalmitis. In some embodiments, the inflammatory disease of the eye is optic neuritis. In some embodiments, the inflammatory disease of the eye is macular degeneration.

In some embodiments, the inflammatory disease of the gums is periodontitis.

In certain embodiments, the inflammatory disease of the skin is sclerodermatitis, sunburn, psoriasis, atopic dermatitis, alopecia areata, or eczema. In certain embodiments, the inflammatory disease of the skin is sclerodermatitis. In certain embodiments, the inflammatory disease of the skin is sunburn. In certain embodiments, the inflammatory disease of the skin is psoriasis. In certain embodiments, the inflammatory disease of the skin is atopic dermatitis. In certain embodiments, the inflammatory disease of the skin is alopecia areata. In certain embodiments, the inflammatory disease of the skin is eczema.

In some embodiments, the inflammatory disease of the joints is arthritis, facet syndrome, tendonitis, bursitis, inflammation of the ligament, synovitis, gout, or systemic lupus erythematosus. In some embodiments, the inflammatory disease of the joints is facet syndrome. In some embodiments, the inflammatory disease of the joints is tendonitis. In some embodiments, the inflammatory disease of the joints is bursitis. In some embodiments, the inflammatory disease of the joints is inflammation of the ligament. In some embodiments, the inflammatory disease of the joints is synovitis. In some embodiments, the inflammatory disease of the joints is gout. In some embodiments, the inflammatory disease of the joints is systemic lupus erythematosus. In some embodiments, the inflammatory disease of the joints is arthritis. In certain embodiments, the arthritis is septic arthritis, rheumatoid arthritis, osteoarthritis, tuberculosis arthritis, leprosy arthritis, or sarcoid arthritis. In certain embodiments, the arthritis is septic arthritis. In certain embodiments, the arthritis is rheumatoid arthritis. In certain embodiments, the arthritis is osteoarthritis. In certain embodiments, the arthritis is tuberculosis arthritis. In certain embodiments, the arthritis is leprosy arthritis. In certain embodiments, the arthritis is sarcoid arthritis.

In some embodiments, the disease or disorder is an autoimmune disease. In certain embodiments, the autoimmune disease is inflammatory bowel disease (IBD), multiple sclerosis (MS), amyotrophic lateral sclerosis (ALS), rheumatoid arthritis (R^{A}), systemic lupus erythematosus, or psoriasis. In some embodiments, the disease or disorder is inflammatory bowel disease (IBD). In some embodiments, the disease or disorder is multiple sclerosis (MS). In some embodiments, the disease or disorder is amyotrophic lateral sclerosis (ALS). In some embodiments, the disease or disorder is rheumatoid arthritis (R^{A}). In some embodiments, the disease or disorder is systemic lupus erythematosus. In some embodiments, the disease or disorder is psoriasis.

In some embodiments, the methods provided herein increase of the activity of a protein/peptide (e.g., cytokine, enzyme, antibody, hormone) activity in a cell, to a level that is statistically significantly higher than an initial level, which may, for example, be a baseline level of enzyme activity. In some embodiments, the term refers to an increase in the level of protein/peptide (e.g., cytokine, enzyme, antibody, hormone) activity to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0,001%, or less than 0,0001% of an initial level, which may, for example, be a baseline level of protein/peptide (e.g., cytokine, enzyme, antibody, hormone) activity.

In some embodiments, the methods provided herein increase of the expression of a protein/peptide (e.g., cytokine, enzyme, antibody, hormone) in a cell, to a level that is statistically significantly higher than an initial level, which may, for example, be a baseline level of expression. In some embodiments, the term refers to an increase in the level of protein/peptide (e.g., cytokine, enzyme, antibody, hormone) expression to a level that is more than 200%, more than 150%, more than 100%, more than 90%, more than 80%, more than 75%, more than 50%, more than 40%, more than 30%, more than 25%, more than 20%, more than 10%, more than 9%, more than 8%, more than 7%, more than 6%, more than 5% of an initial level, which may, for example, be a baseline level of protein/peptide (e.g., cytokine, enzyme, antibody, hormone) expression.

In some embodiments, the methods provided herein increase of the amount of a protein/peptide (e.g., cytokine, enzyme, antibody, hormone) in a cell, to a level that is statistically significantly higher than an initial level, which may, for example, be a baseline level of expression. In some embodiments, the term refers to an increase in the level of protein/peptide (e.g., cytokine, enzyme, antibody, hormone) to a level that more than 200%, more than 150%, more than 100%, more than 90%, more than 80%, more than 75%, more than 50%, more than 40%, more than 30%, more than 25%, more than 20%, more than 10%, more than 9%, more than 8%, more than 7%, more than 6%, more than 5% of an initial level, which may, for example, be a baseline level of protein/peptide (e.g., cytokine, enzyme, antibody, hormone) expression.

### Examples

As used herein, absorption spectral data is in the form of absorbance, Absorbance is expressed in arbitrary units (unless indicated otherwise), and thus, may be excluded, Wavelength is expressed in nm (unless indicate otherwise).

Unless otherwise noted, the pDNA used throughout the examples was either XT-150 or a modified XT-150 plasmid wherein GFP is in place of the IL-10var transgene (same number of base pairs).

### Synthetic Example 1

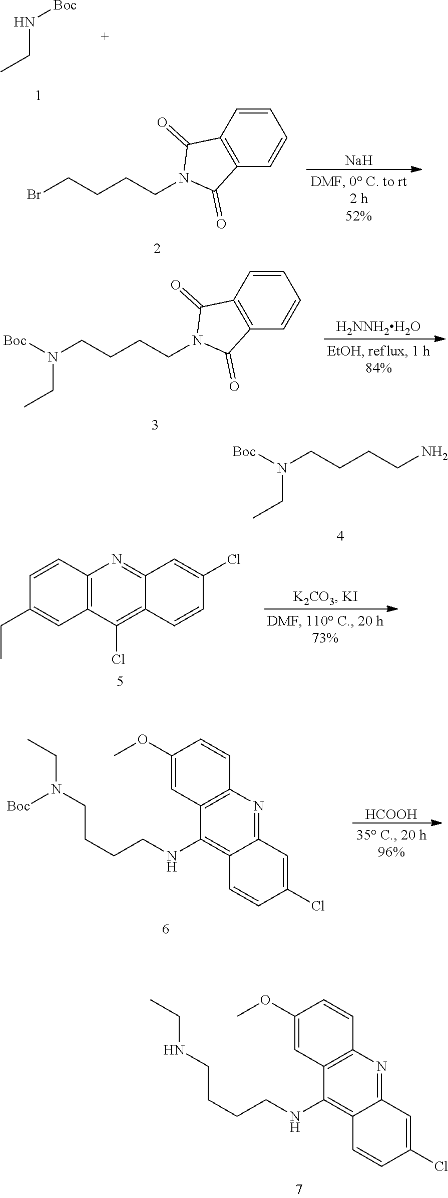

The alkylation of carbamate 1 with phthalimide 2 and subsequent hydrazine deprotection provided 4 in moderate yield, By solvent switching to DMF, which in turn allowed for higher reaction temperatures, the yield was increased to 73%, Upon completion, addition of water and ethyl acetate to the crude reaction mixture facilitated the precipitation of 6 which could then be isolated in high purity by filtration. Further material could be recovered by column chromatography purification. The Boc deprotection of 6 gave diamine 7 in high yield and good purity (50.5 mg),

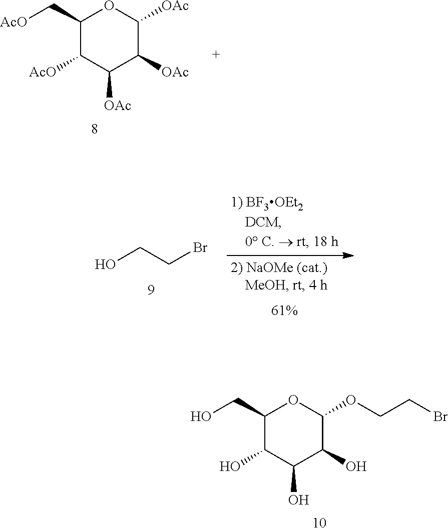

Mannose linker 10 was prepared in two steps from penta-acetyl-D-mannose 8 and bromoethanol 9 in a moderate yield following the previously reported procedure.

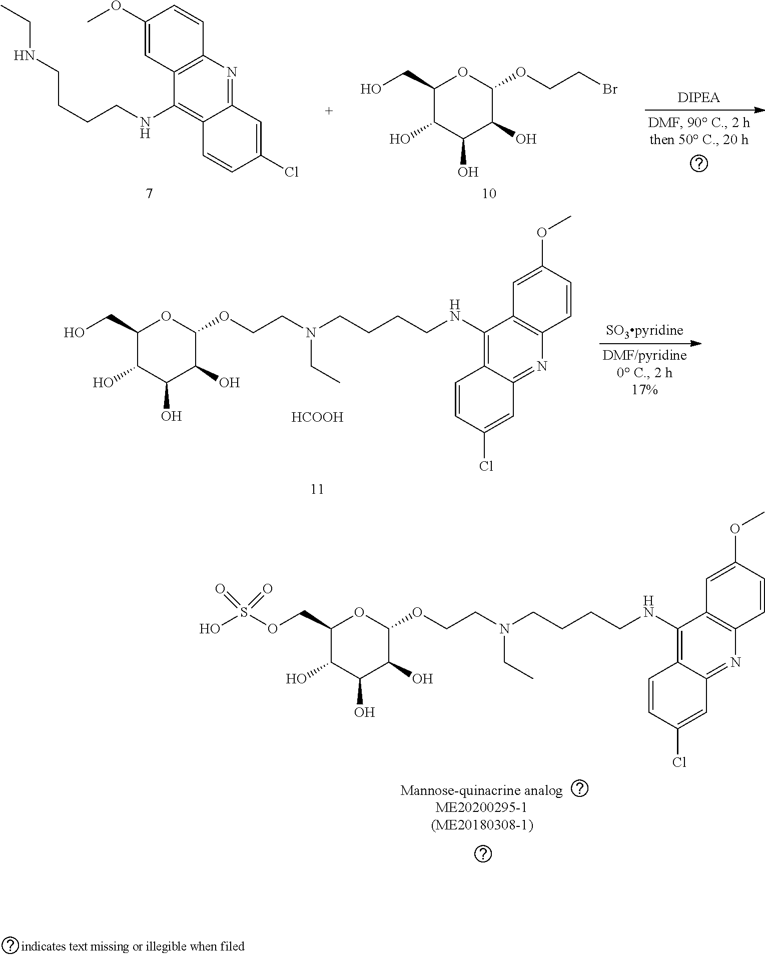 [SMILES: CCNCCCCNc1c2ccc(Cl)cc2nc2ccc(OC)cc12 ; OC[C@H]1O[C@H](OCCBr)[C@@H](O)[C@@H](O)[C@@H]1O ; CCN(CCCCNc1c2ccc(Cl)cc2nc2ccc(OC)cc12)CCO[C@H]1O[C@H](CO)[C@@H](O)[C@H](O)[C@@H]1O ; "HCOOH" ; CCN(CCCCNc1c2ccc(Cl)cc2nc2ccc(OC)cc12)CCO[C@H]1O[C@H](COS(=O)(=O)O)[C@@H](O)[C@H](O)[C@@H]1O]

Alkylation of acridine 7 with mannose linker 10 proceeded smoothly and following purification by acidic RP-HPLC, the formic acid salt of 11 was isolated in 69% yield, Sulfation of 11 with 5 equivalents of sulfur trioxide-pyridine complex was performed on 660 mg scale, Multiple purifications via RP-HPLC were necessary to afford pure Mannoquin 36 (ME20200296-1) (123.9 mg).

### Synthetic Example 2

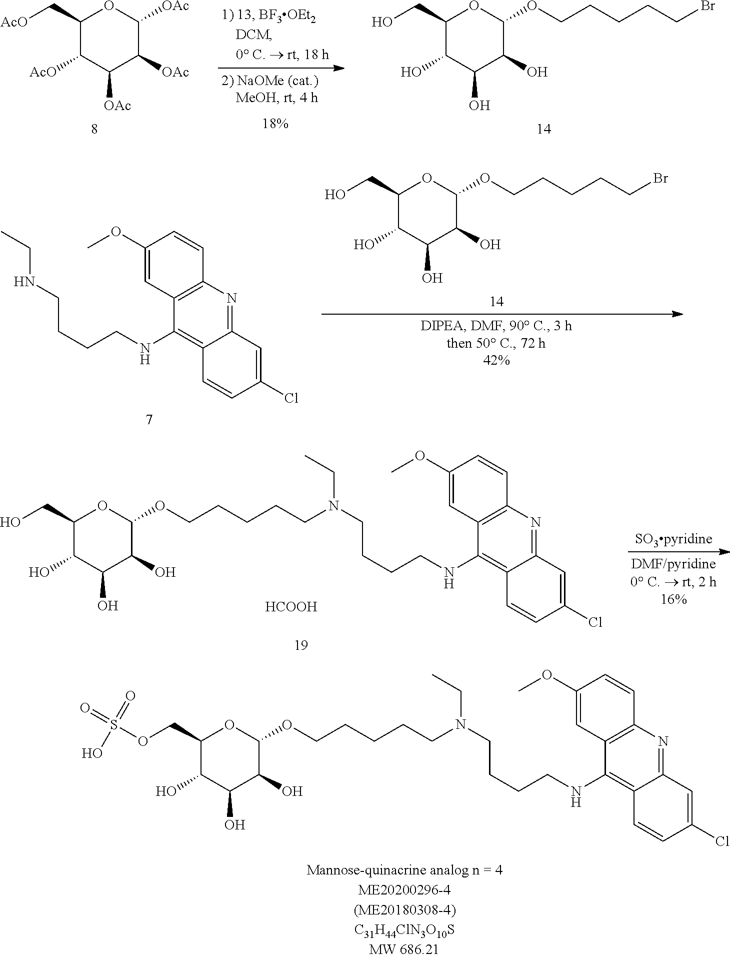

Mannose linker 14 was prepared in 18% yield over two steps from penta-acetyl-D-mannose 8, Mannose linker 14 was obtained in good purity, With 14 in hand, alkylation of acridine 7 was performed, Formic acid salt 19 was isolated in a moderate 42% yield after acidic RP-HPLC purification, Due to the over-sulfation observed for the shorter chain analog, 1 equivalent of sulfur trioxide-pyridine complex was used for the sulfation of 19. The reaction was kept at low temperature and monitored closely by LCMS, Three equivalents of SO₃-pyridine were required to see near complete conversion. The reaction was performed in three batches and these were combined for purification. As described for the n=1 analog, multiple rounds of purification by basic RP-HPLC were required to remove the bis-sulfated side product and residual unreacted 19, though, 180.5 mg of Mannoquin 37 (ME20200296-4) was obtained.

### Synthetic Example 3

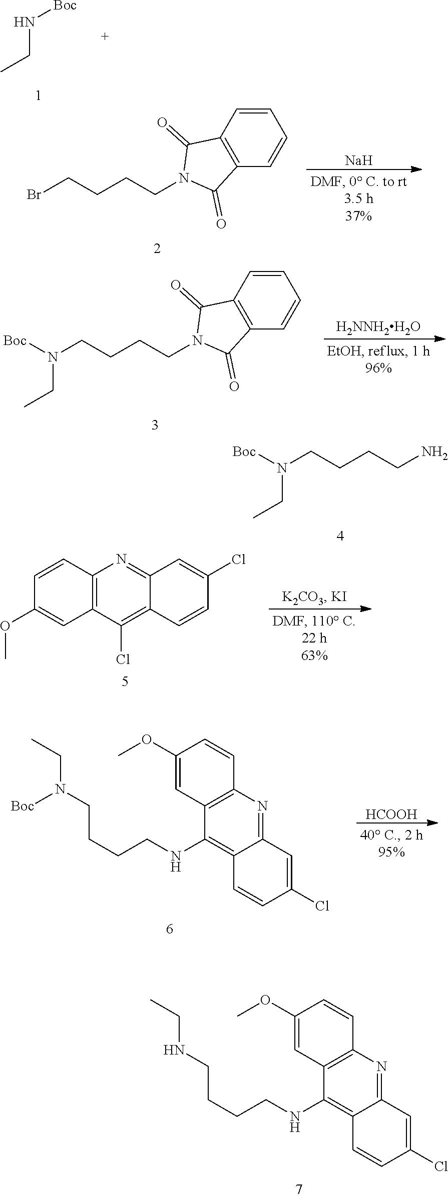

The synthesis started with alkylation of carbamate 1 with bromide 2, affording intermediate 3 in a moderate yield of 37% after purification by flash column chromatography, Next, the phthalimide group was removed by treatment with hydrazine hydrate in ethanol at reflux temperature for 1 h. This gave amine 4 in good yield (96%). The amine was subsequently coupled with acridine 5, affording compound 6 in 63% yield after purification by flash column chromatography. This material was treated with formic acid at 40° C., to remove the Boc group, After the reaction was complete, the mixture was concentrated under reduced pressure. The residue was taken up in water and washed with ethyl acetate in order to remove apolar impurities, Next, the pH was adjusted to pH 10 by dropwise addition of 2M aqueous sodium hydroxide, which caused 7 to precipitate from the aqueous phase. The solids were collected by filtration, washed with water and dried in vacuo, affording 7 in high purity and in high yield (95%),

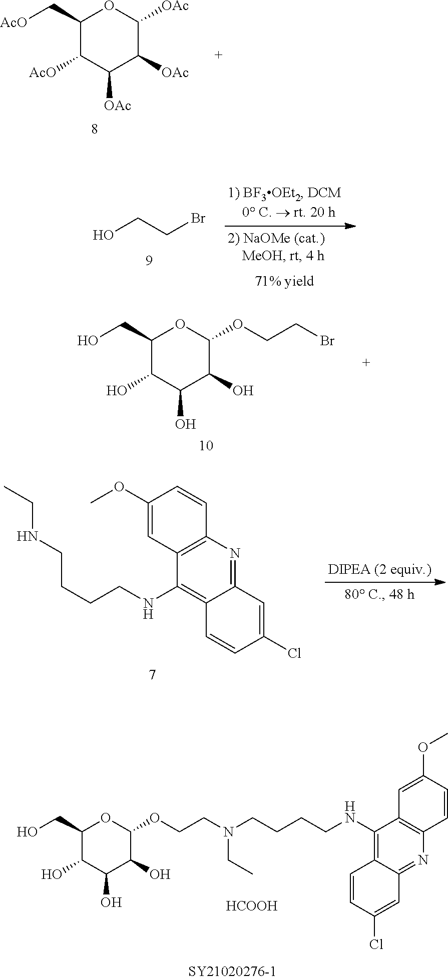

For the preparation of mannose linker 10, the Fisher glycosylation of 8 with 2-bromoethanol (9) was performed, Deacylated mannose linker 10 was then obtained in good yield, after treatment with sodium methoxide in methanol. The crude material was used in the coupling reaction with 7, After a total reaction time of 48 h, LCMS analysis indicated approximately 75% conversion to the target compound. The reaction mixture was purified by acidic preparative MPLC, Two rounds of purification were performed, affording the target compound Mannoquin 38 (SY21020276-1) (173.0 mg) with >95% purity.

### Synthetic Example 4

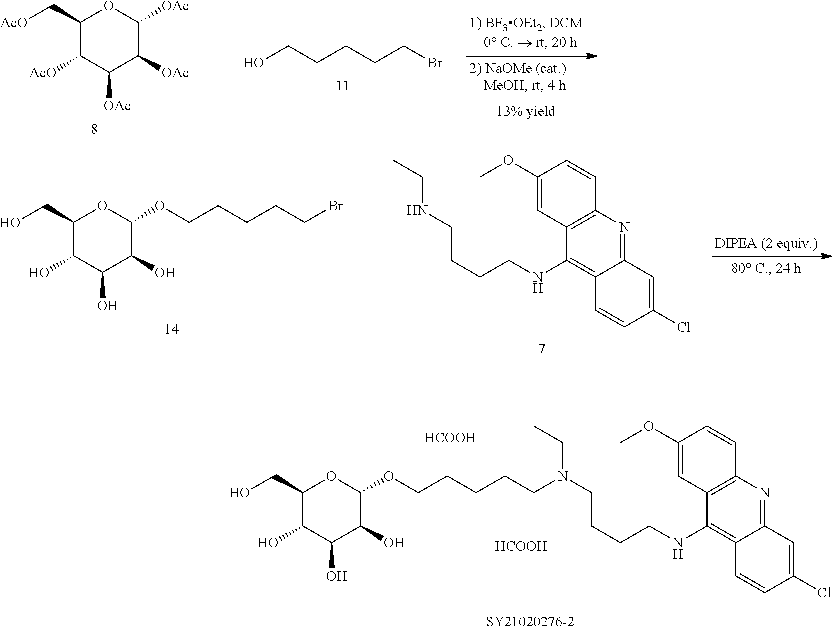

Similarly, 8 was reacted with 5-bromopentanol (11). The product was then treated with catalytic sodium methoxide in order to remove the acetyl protective groups, Additional doses of catalytic NaOMe aided the reaction to completion. The crude material was purified by silica gel flash column chromatography, which afforded 14 as a colorless oil in 13% yield. This material was then coupled with acridine 7, using the reaction conditions that had previously been established, LCMS analysis after a reaction time of 24 h indicated good conversion towards the target compound. The reaction mixture was purified by acidic preparative MPLC and the pooled product fractions were lyophilized. This afforded the target product with a purity of 92.8%, so the material was subjected to another round of purification. In this way, target compound Mannoquin 39 (SY21020276-2) (215.6 mg) was obtained with >95% purity.

### Example 5, Kinetic Solubility Measurements

The test compounds were dissolved in DMSO to a concentration of 10 mM and further diluted to 100 μM in buffer (10 mM PBS, pH 7.4 or potassium phosphate buffer, pH 4) in a 96 well plate. The final DMSO concentration was 1%. Incubations were performed in triplicate. The plates were shaken for 4 h at room temperature in an Eppendorf Thermomixer, After 4 h incubation, the plates were centrifuged for 20 minutes at 3700 rpm, From the supernatant 150 μL was transferred to a new 96 well plate and 50 μL DMSO was added to ensure continued dissolution, Samples were measured on LC-UV with injection volumes of 1 and 4 μL, Peak areas were determined and compared to peak areas obtained using calibration curves of the test compounds in DMSO. The results are depicted in Table 1,

TABLE 1
Kinetic solubility measurements at pH = 7.4 and pH = 4

| | Samples | | | |
|---|---|---|---|---|
| | Solubility μM (n = 3) | | Solubility mg/L (n = 3) | |
| Sample Name | Average (μM) | SE | Average (mg/L) | SE |
| Ketoprofen | >80 | N/A | >20 | N/A |
| Estradiol | 10 | 0.9 | 3 | 0.2 |
| Testosterone | 40 | 1 | 12 | 0.3 |
| Mannoquin 38 | >80* | N/A | >45* | N/A |
| (SY21020276-1) | | | | |
| pH 7.4 | | | | |
| Mannoquin 39 | >80 | N/A | >48 | N/A |
| (SY21020276-2) | | | | |
| pH 7.4 | | | | |
| Mannoquin 38 | >80* | N/A | >45* | N/A |
| (SY21020276-1) | | | | |
| pH 4 | | | | |
| Mannoquin 39 | >80 | N/A | >48 | N/A |
| (SY21020276-2) | | | | |
| pH 4 | | | | |

*Measured using basic LC conditions

The measurements showed that at both pH values, the target compounds were fully soluble at the highest measured concentration (i.e. solubility >80 μM), This resulted in a solubility of >45 mg/L for SY21020276-1 (Mannoquin 38) and of >48 mg/L for SY21020276-2 (Mannoquin 39).

### Example 6: Quinacrine-DNA Binding Saturation Studies

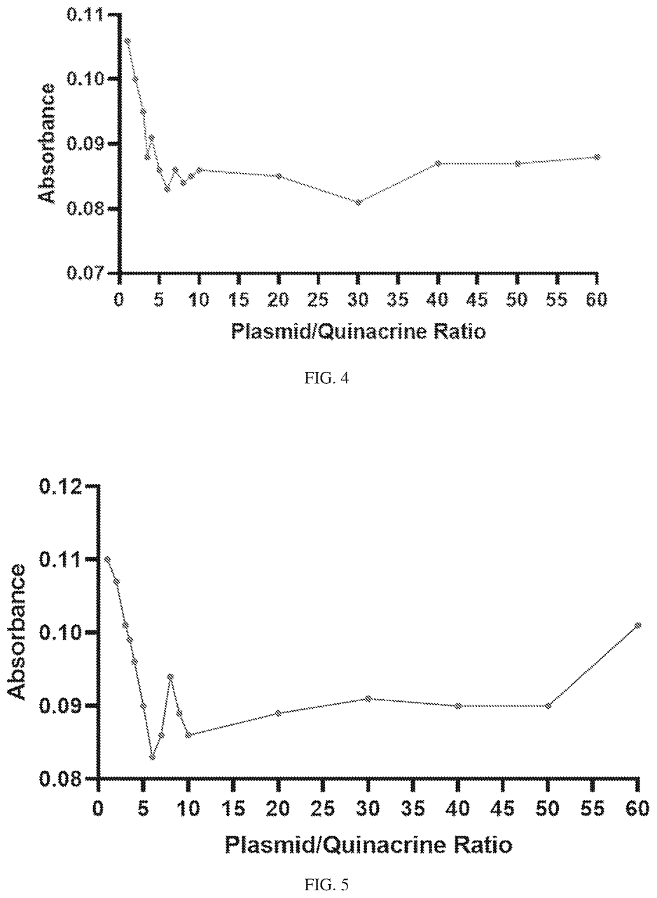

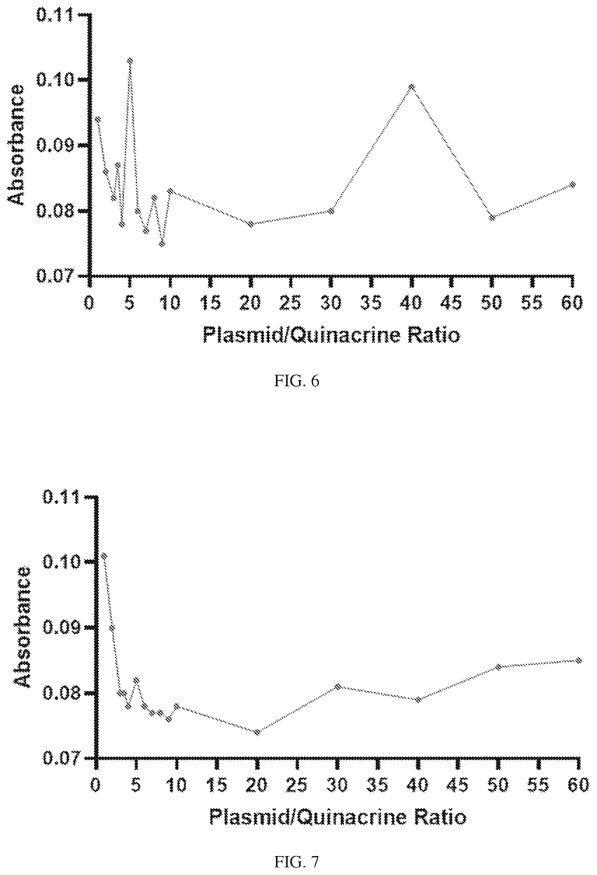

To study the saturation point for each mannoquin compound, plasmid/mannoquin binding saturation studies (e.g., FIGS. 4 to 7) were carried out, Plasmid was provided as a 5.4 mg/mL stock solution and was diluted to 1 mg/mb or 0.1 mg/mL with water. The reaction volume was 200 μL, the mannoquin concentration was approximately 30 JAM (0.1 mg/mL), the DNA concentration ranged from 0 to 0.9 mg/mL, and the DNA/mannoquin ratio ranged from 0 to 90, Absorption spectra were collected from 340 to 550 nm, Results appear in Tables 2 to 5.

For Mannoquin 36, plasmid saturation occurs around a ratio of 30/40.

TABLE 2
Plasmid/Quinacrine Saturation for Mannoquin 36

| Plasmid/Mannoquin Ratio | Abs. @ 424 nm | Abs. @ 430 nm |
|---|---|---|
| 0 | 0.109 | 0.102 |
| 1 | 0.106 | 0.102 |
| 2 | 0.1 | 0.099 |
| 3 | 0.095 | 0.096 |
| 3.5 | 0.088 | 0.09 |
| 4 | 0.091 | 0.093 |
| 5 | 0.086 | 0.089 |
| 6 | 0.083 | 0.087 |
| 7 | 0.086 | 0.089 |
| 8 | 0.084 | 0.087 |
| 9 | 0.085 | 0.089 |
| 10 | 0.086 | 0.09 |
| 20 | 0.085 | 0.089 |
| 30 | 0.081 | 0.084 |
| 40 | 0.087 | 0.092 |
| 50 | 0.087 | 0.091 |
| 60 | 0.088 | 0.093 |
| 70 | 0.096 | 0.101 |
| 80 | 0.088 | 0.092 |
| 90 | 0.09 | 0.094 |

For Mannoquin 37, plasmid saturation occurs around a ratio of 10/20,

TABLE 3
Plasmid/Quinacrine Saturation for Mannoquin 37

| Plasmid/Mannoquin Ratio | Abs. @ 424 nm | Abs. @ 430 nm |
|---|---|---|
| 0 | 0.119 | 0.112 |
| 1 | 0.11 | 0.106 |
| 2 | 0.107 | 0.105 |
| 3 | 0.101 | 0.101 |
| 3.5 | 0.099 | 0.1 |
| 4 | 0.096 | 0.098 |
| 5 | 0.09 | 0.093 |
| 6 | 0.083 | 0.087 |
| 7 | 0.086 | 0.09 |
| 8 | 0.094 | 0.098 |
| 9 | 0.089 | 0.094 |
| 10 | 0.086 | 0.089 |
| 20 | 0.089 | 0.094 |
| 30 | 0.091 | 0.096 |
| 40 | 0.09 | 0.094 |
| 50 | 0.09 | 0.094 |
| 60 | 0.101 | 0.107 |
| 70 | 0.095 | 0.1 |
| 80 | 0.112 | 0.116 |
| 90 | 0.097 | 0.101 |

For Mannoquin 38, results show possible saturation occurring after a ratio of 50:1,

TABLE 4
Plasmid/Quinacrine Saturation for Mannoquin 38

| Plasmid/Mannoquin 38 Ratio | Abs. @ 424 nm | Abs. @ 430 nm |
|---|---|---|
| 0 | 0.106 | 0.1 |
| 1 | 0.094 | 0.091 |
| 2 | 0.086 | 0.087 |
| 3 | 0.082 | 0.086 |
| 3.5 | 0.087 | 0.091 |
| 4 | 0.078 | 0.082 |
| 5 | 0.103 | 0.106 |
| 6 | 0.08 | 0.084 |
| 7 | 0.077 | 0.082 |
| 8 | 0.082 | 0.086 |
| 9 | 0.075 | 0.079 |
| 10 | 0.083 | 0.086 |
| 20 | 0.078 | 0.082 |
| 30 | 0.08 | 0.084 |
| 40 | 0.099 | 0.103 |
| 50 | 0.079 | 0.082 |
| 60 | 0.084 | 0.088 |
| 70 | 0.08 | 0.083 |
| 80 | 0.099 | 0.102 |
| 90 | 0.085 | 0.089 |

For Mannoquin 39, plasmid saturation occurs at a ratio of 30/40,

TABLE 5
Plasmid/Quinacrine Saturation for Mannoquin 39

| Plasmid/Mannoquin 39 Ratio | Abs. @ 424 nm | Abs. @ 430 nm |
|---|---|---|
| 0 | 0.106 | 0.1 |
| 1 | 0.101 | 0.099 |
| 2 | 0.09 | 0.09 |
| 3 | 0.08 | 0.083 |
| 3.5 | 0.08 | 0.084 |
| 4 | 0.078 | 0.082 |
| 5 | 0.082 | 0.086 |
| 6 | 0.078 | 0.082 |
| 7 | 0.077 | 0.081 |
| 8 | 0.077 | 0.081 |
| 9 | 0.076 | 0.08 |
| 10 | 0.078 | 0.081 |
| 20 | 0.074 | 0.078 |
| 30 | 0.081 | 0.085 |
| 40 | 0.079 | 0.082 |
| 50 | 0.084 | 0.088 |
| 60 | 0.085 | 0.089 |
| 70 | 0.084 | 0.088 |
| 80 | 0.081 | 0.084 |
| 90 | 0.083 | 0.087 |

Overall, these results indicate Mannoquin 37 (ME20200296-4) shows the strongest affinity for DNA, with a saturation point occurring at a plasmid:mannoquin ratio of 10-20, Mannoquin 36 (ME20200296-1) and Mannoquin 39 (SY21020276-2) show similar affinity for DNA, with a saturation point occurring at a plasmid:mannoquin ratio of 30-40.

### Example 7, Plasmid/Mannoquin Stability Studies

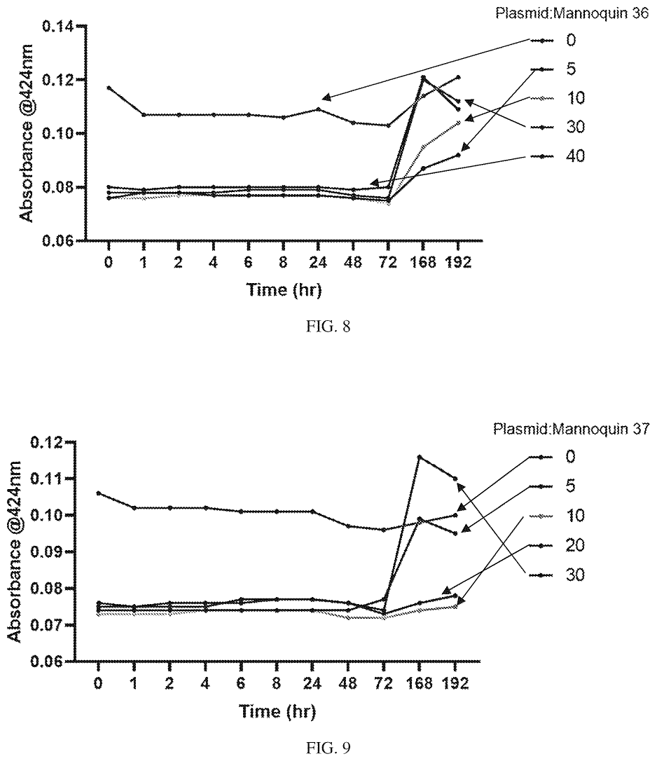

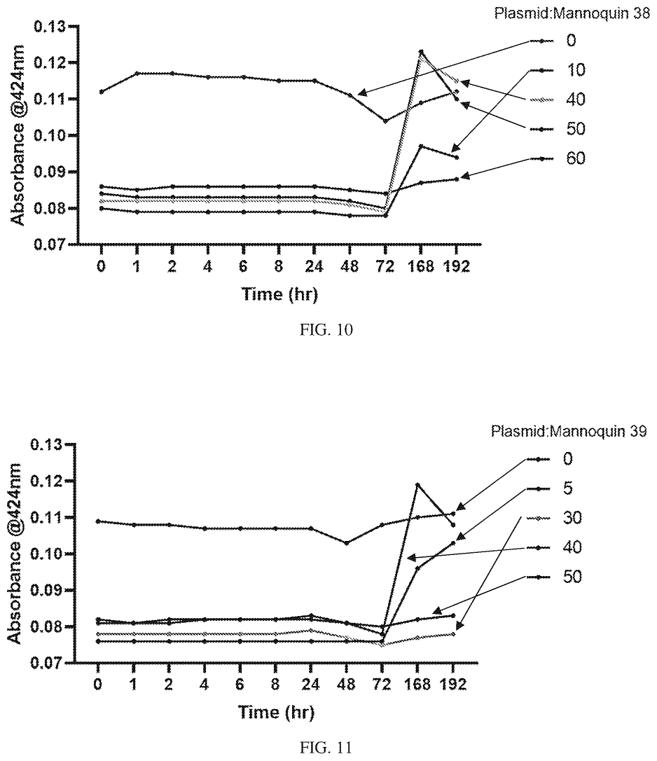

The stability of the plasmid/mannoquin complex over time was studied, Plasmid was provided as 5.4 mg/mL stock and was diluted to 1 mg/mL with water. The reaction volume was 200 μL, the mannoquin concentration was approximately 30 μM (0.1 mg/mL), the DNA concentration ranged from 0 to 0.6 mg/mL, and the DNA/mannoquin ratio was as follows: Mannoquin 36: 0, 5, 10, 30, 40; Mannoquin 37: 0, 5, 10, 20, 30; Mannoquin 38: 0, 10, 40, 50, 60; and Mannoquin 39: 0, 5, 30, 40, 50, Absorption spectra from 340 to 550 nm were collected on the indicated days/times, Results, in the form of absorbance at 424 nm, appear in Tables 6 to 9, See also FIGS. 8 to 11.

Table 6 shows that plasmid and Mannoquin 36 remain complexed after 3 days at the saturation point (ratio of 30/40), All complexes appear to dissociate after 7 days,

TABLE 6
Plasmid/Quinacrine Stability for Mannoquin 36 at 424 nm

Plasmid/ Mannoquin 36 Ratios

| Time Point (hrs) | 0 | 5 | 10 | 30 | 40 |
|---|---|---|---|---|---|
| T0 | 0.117 | 0.076 | 0.076 | 0.078 | 0.080 |
| T1 | 0.107 | 0.078 | 0.076 | 0.078 | 0.079 |
| T2 | 0.107 | 0.078 | 0.077 | 0.078 | 0.080 |
| T4 | 0.107 | 0.077 | 0.077 | 0.078 | 0.080 |
| T6 | 0.107 | 0.077 | 0.077 | 0.079 | 0.080 |
| T8 | 0.106 | 0.077 | 0.077 | 0.079 | 0.080 |
| T24 | 0.109 | 0.077 | 0.077 | 0.079 | 0.080 |
| T48 | 0.104 | 0.076 | 0.076 | 0.077 | 0.079 |
| T72 | 0.103 | 0.075 | 0.074 | 0.076 | 0.080 |
| T168 | 0.114 | 0.087 | 0.095 | 0.120 | 0.121 |
| T192 | 0.121 | 0.092 | 0.104 | 0.112 | 0.109 |

Table 7 shows plasmid and Mannoquin 37 appear to remain complexed after 8 days at the saturation point (ratio of 10/20), Lower and higher ratios appear to be less stable,

TABLE 7
Plasmid/Quinacrine Stability for Mannoquin 37 at 424 nm

Plasmid/ Mannoquin 37 Ratios

| Time Point (hrs) | 0 | 5 | 10 | 20 | 30 |
|---|---|---|---|---|---|
| T0 | 0.106 | 0.074 | 0.073 | 0.075 | 0.076 |
| T1 | 0.102 | 0.074 | 0.073 | 0.075 | 0.075 |
| T2 | 0.102 | 0.074 | 0.073 | 0.075 | 0.076 |
| T4 | 0.102 | 0.074 | 0.074 | 0.075 | 0.076 |
| T6 | 0.101 | 0.074 | 0.074 | 0.077 | 0.076 |
| T8 | 0.101 | 0.074 | 0.074 | 0.077 | 0.077 |
| T24 | 0.101 | 0.074 | 0.074 | 0.077 | 0.077 |
| T48 | 0.097 | 0.074 | 0.072 | 0.076 | 0.076 |
| T72 | 0.096 | 0.077 | 0.072 | 0.073 | 0.074 |
| T168 | 0.098 | 0.099 | 0.074 | 0.076 | 0.116 |
| T192 | 0.100 | 0.095 | 0.075 | 0.078 | 0.110 |

Table 8 shows that plasmid and Mannoquin 38 appear to remain complexed after 8 days at a ratio of 60:1, Lower ratios appear to be less stable and dissociate after 7 days,

TABLE 8
Plasmid/Quinacrine Stability for Mannoquin 38 at 424 nm

| | |
|---|---|
| Time Point | Plasmid/Mannoquin 38 Ratios |

| (hrs) | 0 | 10 | 40 | 50 | 60 |
|---|---|---|---|---|---|
| T0 | 0.112 | 0.080 | 0.082 | 0.084 | 0.086 |
| T1 | 0.117 | 0.079 | 0.082 | 0.083 | 0.085 |
| T2 | 0.117 | 0.079 | 0.082 | 0.083 | 0.086 |
| T4 | 0.116 | 0.079 | 0.082 | 0.083 | 0.086 |
| T6 | 0.116 | 0.079 | 0.082 | 0.083 | 0.086 |
| T8 | 0.115 | 0.079 | 0.082 | 0.083 | 0.086 |
| T24 | 0.115 | 0.079 | 0.082 | 0.083 | 0.086 |
| T48 | 0.111 | 0.078 | 0.081 | 0.082 | 0.085 |
| T72 | 0.104 | 0.078 | 0.079 | 0.080 | 0.084 |
| T168 | 0.109 | 0.097 | 0.121 | 0.123 | 0.087 |
| T192 | 0.112 | 0.094 | 0.115 | 0.110 | 0.088 |

Table 9 shows that plasmid and Mannoquin 39 appear to remain complexed after 8 days at the saturation point (ratio of 30) and above the saturation point (ratio of 50). At a ratio of 40, dissociation of the complex appears to occur after 7 days,

TABLE 9
Plasmid/Quinacrine Stability for Mannoquin 39 at 424 nm

| | |
|---|---|
| Time Point | Plasmid/ Mannoquin 39 Ratios |

| (hrs) | 0 | 5 | 30 | 40 | 50 |
|---|---|---|---|---|---|
| T0 | 0.109 | 0.076 | 0.078 | 0.082 | 0.081 |
| T1 | 0.108 | 0.076 | 0.078 | 0.081 | 0.081 |
| T2 | 0.108 | 0.076 | 0.078 | 0.082 | 0.081 |
| T4 | 0.107 | 0.076 | 0.078 | 0.082 | 0.082 |
| T6 | 0.107 | 0.076 | 0.078 | 0.082 | 0.082 |
| T8 | 0.107 | 0.076 | 0.078 | 0.082 | 0.082 |
| T24 | 0.107 | 0.076 | 0.079 | 0.083 | 0.082 |
| T48 | 0.103 | 0.076 | 0.077 | 0.081 | 0.081 |
| T72 | 0.108 | 0.076 | 0.075 | 0.078 | 0.080 |
| T168 | 0.110 | 0.096 | 0.077 | 0.119 | 0.082 |
| T192 | 0.111 | 0.103 | 0.078 | 0.108 | 0.083 |

Overall, these stability studies show that Mannoquin/DNA complexes are stable for a minimum of 72 hours with Mannoquin 36 exhibiting the lowest stability around the saturation point and Mannoquin 37 exhibiting the highest stability around the saturation point.

### Example 8, Plasmid/Mannoquin Stability Studies with Varying pH

The stability of the plasmid/mannoquin complex over time (day 1 to 25) and at varying pH (2, 4, 6, 7.4, and 8) was studied, Plasmid was provided as 13.0 mg/mL stock solution and was diluted to 1 mg/mL with water. The reaction volume was 200 LL, the mannoquin concentration was approximately 30 μM (0.1 mg/mL), and DNA concentration ranged from 0 to 0.6 mg/mL with:
DNA:Mannoquin 36 ratios ranging from 0, 10, 30, and 40, with PBS buffer to generate a pH of 2, 4, 6, 7.4, and 8 (in PBS, adjusted with HCl or NaOH);
DNA:Mannoquin 37 ratios ranging from 0, 5, 10, 20, and 30, with PBS buffer to generate a pH of 2, 4, 6, 7.4, and 8 (in PBS, adjusted with HCl or NaOH);
DNA:Mannoquin 38 ratios ranging from 0, 10, 40, 50, and 60, with PBS buffer to generate a pH of 2, 4, 6, 7.4, and 8 (in PBS, adjusted with HCl or NaOH); and
DNA:Mannoquin 39 ratios ranging from 0, 5, 10, 30, and 40, with PBS buffer to generate a pH of 2, 4, 6, 7.4, and 8 (in PBS, adjusted with HCl or NaOH).
   Absorption spectra were collected on the indicated days, Results, in the form of absorbance at 424 nm, appear in Tables 10 to 30.

TABLE 10
Effect of pH on Stability of DNA-Mannoquin Complex

| Ratio | | Absorbance @ A424 nm | | | | | | | | | |
|---|---|---|---|---|---|---|---|---|---|---|---|
| DNA:Man | | pH 2 | | pH 4 | | pH 6 | | PH 7.4 | | pH 8.0 | |
| Time (Days) | | 0 | 25/26 | 0 | 25/26 | 0 | 25/26 | 0 | 25/26 | 0 | 25/26 |
| Mannoquin | 0 | 0.124 | 0.057 | 0.121 | 0.115 | 0.113 | 0.176 | 0.126 | 0.107 | 0.088 | 0.108 |
| 36 | 30 | 0.116 | 0.114 | 0.083 | 0.128 | 0.080 | 0.121 | 0.088 | 0.088 | 0.064 | 0.088 |
| Mannoquin | 0 | 0.120 | 0.110 | 0.113 | 0.108 | 0.113 | 0.146 | 0.114 | 0.099 | 0.088 | 0.123 |
| 37 | 20 | 0.114 | 0.112 | 0.096 | 0.126 | 0.083 | 0.099 | 0.078 | 0.077 | 0.069 | 0.102 |
| Mannoquin | 0 | 0.125 | 0.133 | 0.126 | 0.118 | 0.119 | 0.140 | 0.121 | 0.107 | 0.114 | 0.151 |
| 38 | 50 | 0.099 | 0.109 | 0.084 | 0.146 | 0.082 | 0.094 | 0.084 | 0.086 | 0.080 | 0.124 |
| Mannoquin | 0 | 0.119 | 0.115 | 0.117 | 0.058 | 0.117 | 0.139 | 0.111 | 0.099 | 0.106 | 0.151 |
| 39 | 30 | 0.108 | 0.112 | 0.079 | 0.127 | 0.081 | 0.098 | 0.078 | 0.076 | N/A | 0.050 |

TABLE 11
Effect of pHI on Stability of DNA-Mannoquin 36 Complex at pH 2

| | |
|---|---|
| Time Point | Plasmid/Mannoquin 36 Ratios |

| (days) | 0 | 5 | 10 | 30 | 40 |
|---|---|---|---|---|---|
| D 0 | 0.124 | 0.121 | 0.120 | 0.116 | 0.114 |
| D 1 | 0.123 | 0.120 | 0.135 | 0.116 | 0.113 |
| D 2 | 0.145 | 0.119 | 0.134 | 0.116 | 0.113 |
| D 3 | 0.139 | 0.119 | 0.134 | 0.116 | 0.114 |
| D 4 | 0.121 | 0.118 | 0.130 | 0.115 | 0.114 |
| D 7 | 0.119 | 0.117 | 0.124 | 0.115 | 0.113 |
| D 8 | 0.118 | 0.117 | 0.125 | 0.115 | 0.113 |
| D 9 | 0.122 | 0.117 | 0.125 | 0.117 | 0.114 |
| D 10 | 0.122 | 0.117 | 0.117 | 0.115 | 0.114 |
| D 11 | 0.124 | 0.117 | 0.117 | 0.115 | 0.116 |
| D 14 | 0.120 | 0.123 | 0.117 | 0.116 | 0.114 |
| D 15 | 0.123 | 0.117 | 0.117 | 0.115 | 0.115 |
| D 16 | 0.121 | 0.116 | 0.116 | 0.115 | 0.117 |
| D 18 | 0.107 | 0.116 | 0.116 | 0.115 | 0.119 |
| D 21 | 0.087 | 0.128 | 0.117 | 0.115 | 0.119 |
| D 22 | 0.074 | 0.117 | 0.116 | 0.115 | 0.119 |
| D 23 | 0.061 | 0.116 | 0.116 | 0.115 | 0.119 |
| D 24 | 0.057 | 0.116 | 0.116 | 0.114 | 0.119 |
| D 25 | 0.057 | 0.115 | 0.115 | 0.114 | 0.118 |

TABLE 12
Effect of pH on Stability of DNA-Mannoquin 36 Complex at pH 4

| | |
|---|---|
| Time Point | Plasmid/Mannoquin 36 Ratios |

| (days) | 0 | 5 | 10 | 30 | 40 |
|---|---|---|---|---|---|
| D 0 | 0.121 | 0.108 | 0.096 | 0.083 | 0.089 |
| D 1 | 0.125 | 0.106 | 0.096 | 0.083 | 0.086 |
| D 2 | 0.124 | 0.105 | 0.095 | 0.083 | 0.086 |
| D 3 | 0.123 | 0.104 | 0.096 | 0.086 | 0.086 |
| D 4 | 0.129 | 0.104 | 0.094 | 0.092 | 0.087 |
| D 7 | 0.128 | 0.117 | 0.093 | 0.106 | 0.098 |
| D 8 | 0.127 | 0.117 | 0.092 | 0.107 | 0.103 |
| D 9 | 0.132 | 0.102 | 0.092 | 0.109 | 0.103 |
| D 10 | 0.121 | 0.104 | 0.092 | 0.112 | 0.106 |
| D 11 | 0.119 | 0.104 | 0.092 | 0.115 | 0.109 |
| D 14 | 0.119 | 0.103 | 0.092 | 0.115 | 0.109 |
| D 15 | 0.118 | 0.102 | 0.092 | 0.116 | 0.108 |
| D 16 | 0.118 | 0.103 | 0.092 | 0.122 | 0.116 |
| D 18 | 0.117 | 0.102 | 0.092 | 0.122 | 0.113 |
| D 21 | 0.116 | 0.102 | 0.092 | 0.123 | 0.114 |
| D 22 | 0.116 | 0.102 | 0.091 | 0.125 | 0.120 |
| D 23 | 0.116 | 0.101 | 0.091 | 0.126 | 0.119 |
| D 24 | 0.115 | 0.101 | 0.091 | 0.127 | 0.117 |
| D 25 | 0.115 | 0.101 | 0.100 | 0.128 | 0.118 |

TABLE 13
Effect of pH on Stability of DNA-Mannoquin 36 Complex at pH 6

| | |
|---|---|
| Time Point | Plasmid/Mannoquin 36 Ratios |

| (days) | 0 | 5 | 10 | 30 | 40 |
|---|---|---|---|---|---|
| D 0 | 0.113 | 0.097 | 0.089 | 0.080 | 0.080 |
| D 1 | 0.114 | 0.102 | 0.091 | 0.081 | 0.083 |
| D 2 | 0.117 | 0.108 | 0.122 | 0.084 | 0.083 |
| D 3 | 0.127 | 0.112 | 0.131 | 0.090 | 0.087 |
| D 4 | 0.126 | 0.113 | 0.131 | 0.091 | 0.089 |
| D 7 | 0.126 | 0.114 | 0.134 | 0.089 | 0.095 |
| D 8 | 0.133 | 0.111 | 0.123 | 0.087 | 0.089 |
| D 9 | 0.135 | 0.113 | 0.106 | 0.158 | 0.094 |
| D 10 | 0.133 | 0.114 | 0.112 | 0.161 | 0.101 |
| D 11 | 0.136 | 0.115 | 0.112 | 0.171 | 0.095 |
| D 14 | 0.138 | 0.123 | 0.113 | 0.100 | 0.103 |
| D 15 | 0.138 | 0.122 | 0.112 | 0.101 | 0.096 |
| D 16 | 0.143 | 0.120 | 0.114 | 0.106 | 0.099 |
| D 18 | 0.159 | 0.117 | 0.121 | 0.118 | 0.102 |
| D 21 | 0.153 | 0.113 | 0.120 | 0.113 | 0.103 |
| D 22 | 0.157 | 0.150 | 0.111 | 0.119 | 0.096 |
| D 23 | 0.163 | 0.142 | 0.123 | 0.119 | 0.097 |
| D 24 | 0.171 | 0.142 | 0.131 | 0.115 | 0.094 |
| D 25 | 0.176 | 0.145 | 0.139 | 0.121 | 0.099 |

TABLE 14
Efect of pH on Stability of DNA-Mannoquin 36 Complex at pH 8

| | |
|---|---|
| Time Point | Plasmid/Mannoquin 36 Ratios |

| (days) | 0 | 5 | 10 | 30 | 40 |
|---|---|---|---|---|---|
| D 0 | 0.088 | 0.078 | 0.070 | 0.064 | 0.065 |
| D 1 | 0.087 | 0.076 | 0.068 | 0.065 | 0.065 |
| D 2 | 0.087 | 0.076 | 0.068 | 0.065 | 0.065 |
| D 3 | 0.087 | 0.076 | 0.070 | 0.065 | 0.065 |
| D 4 | 0.088 | 0.077 | 0.071 | 0.066 | 0.067 |
| D 7 | 0.104 | 0.088 | 0.088 | 0.082 | 0.080 |
| D 8 | 0.108 | 0.091 | 0.092 | 0.088 | 0.085 |
| D 9 | 0.108 | 0.091 | 0.091 | 0.084 | 0.079 |
| D 10 | 0.118 | 0.090 | 0.090 | 0.096 | 0.081 |
| D 11 | 0.115 | 0.091 | 0.094 | 0.097 | 0.088 |
| D 14 | 0.101 | 0.087 | 0.086 | 0.091 | 0.086 |
| D 15 | 0.100 | 0.087 | 0.089 | 0.092 | 0.087 |
| D 16 | 0.106 | 0.090 | 0.098 | 0.098 | 0.086 |
| D 18 | 0.098 | 0.088 | 0.090 | 0.094 | 0.088 |
| D 21 | 0.104 | 0.103 | 0.096 | 0.089 | 0.116 |
| D 22 | 0.105 | 0.107 | 0.095 | 0.089 | 0.102 |
| D 23 | 0.108 | 0.103 | 0.094 | 0.085 | 0.093 |
| D 24 | 0.105 | 0.100 | 0.093 | 0.086 | 0.087 |
| D 25 | 0.108 | 0.096 | 0.099 | 0.088 | 0.087 |

TABLE 15
Effect of pH on Stability of DNA-
Mannoquin 36 Complex at pH 7.4

| | |
|---|---|
| Time Point | Plasmid/Mannoquin 36 Ratios |

| (days) | 0 | 5 | 10 | 30 | 40 |
|---|---|---|---|---|---|
| T0 | 0.126 | 0.104 | 0.096 | 0.088 | 0.088 |
| T6 | 0.118 | 0.102 | 0.094 | 0.088 | 0.085 |
| T7 | 0.117 | 0.102 | 0.094 | 0.088 | 0.085 |
| T8 | 0.117 | 0.108 | 0.094 | 0.088 | 0.085 |
| T9 | 0.108 | 0.115 | 0.091 | 0.086 | 0.084 |
| T12 | 0.112 | 0.111 | 0.091 | 0.087 | 0.084 |
| T13 | 0.113 | 0.110 | 0.091 | 0.087 | 0.085 |
| T14 | 0.111 | 0.110 | 0.092 | 0.087 | 0.085 |

TABLE 16
Effect of pH on Stability of DNA-Mannoquin 37 Complex at pH 2

| | |
|---|---|
| Time Point | Plasmid/Mannoquin 37 Ratios |

| (days) | 0 | 5 | 10 | 20 | 30 |
|---|---|---|---|---|---|
| D 0 | 0.120 | 0.115 | 0.114 | 0.114 | 0.112 |
| D 1 | 0.119 | 0.116 | 0.137 | 0.113 | 0.111 |
| D 2 | 0.119 | 0.116 | 0.136 | 0.112 | 0.111 |
| D 3 | 0.119 | 0.115 | 0.135 | 0.112 | 0.111 |
| D 4 | 0.118 | 0.116 | 0.112 | 0.111 | 0.111 |
| D 7 | 0.119 | 0.115 | 0.111 | 0.111 | 0.111 |
| D 8 | 0.118 | 0.120 | 0.111 | 0.111 | 0.110 |
| D 9 | 0.118 | 0.116 | 0.111 | 0.116 | 0.110 |
| D 10 | 0.118 | 0.116 | 0.111 | 0.111 | 0.110 |
| D 11 | 0.118 | 0.115 | 0.111 | 0.111 | 0.110 |
| D 14 | 0.118 | 0.115 | 0.111 | 0.111 | 0.111 |
| D 15 | 0.118 | 0.115 | 0.111 | 0.111 | 0.111 |
| D 16 | 0.119 | 0.115 | 0.111 | 0.110 | 0.110 |
| D 18 | 0.116 | 0.115 | 0.111 | 0.116 | 0.110 |
| D 21 | 0.114 | 0.115 | 0.111 | 0.117 | 0.111 |
| D 22 | 0.112 | 0.115 | 0.111 | 0.116 | 0.111 |
| D 23 | 0.112 | 0.115 | 0.111 | 0.116 | 0.110 |
| D 24 | 0.110 | 0.115 | 0.111 | 0.116 | 0.110 |
| D 25 | 0.110 | 0.116 | 0.111 | 0.112 | 0.110 |

TABLE 17
Effect of pH on Stability of DNA-Mannoquin 37 Complex at pH 4

| | |
|---|---|
| Time Point | Plasmid/Mannoquin 37 Ratios |

| (days) | 0 | 5 | 10 | 20 | 30 |
|---|---|---|---|---|---|
| D 0 | 0.113 | 0.097 | 0.096 | 0.096 | 0.087 |
| D 1 | 0.111 | 0.097 | 0.089 | 0.081 | 0.081 |
| D 2 | 0.110 | 0.096 | 0.089 | 0.099 | 0.083 |
| D 3 | 0.109 | 0.096 | 0.089 | 0.106 | 0.085 |
| D 4 | 0.116 | 0.097 | 0.089 | 0.084 | 0.089 |
| D 7 | 0.111 | 0.095 | 0.088 | 0.090 | 0.114 |
| D 8 | 0.111 | 0.094 | 0.088 | 0.094 | 0.118 |
| D 9 | 0.111 | 0.094 | 0.087 | 0.097 | 0.124 |
| D 10 | 0.111 | 0.094 | 0.096 | 0.100 | 0.129 |
| D 11 | 0.111 | 0.105 | 0.095 | 0.103 | 0.131 |
| D 14 | 0.111 | 0.094 | 0.096 | 0.109 | 0.133 |
| D 15 | 0.110 | 0.091 | 0.095 | 0.112 | 0.133 |
| D 16 | 0.110 | 0.099 | 0.087 | 0.123 | 0.136 |
| D 18 | 0.110 | 0.091 | 0.087 | 0.121 | 0.135 |
| D 21 | 0.114 | 0.091 | 0.087 | 0.123 | 0.134 |
| D 22 | 0.110 | 0.090 | 0.086 | 0.125 | 0.136 |
| D 23 | 0.109 | 0.091 | 0.086 | 0.125 | 0.136 |
| D 24 | 0.108 | 0.091 | 0.087 | 0.126 | 0.136 |
| D 25 | 0.108 | 0.091 | 0.086 | 0.126 | 0.138 |

TABLE 18
Effect of pH on Stability of DNA-Mannoquin 37 Complex at pH 6

| | |
|---|---|
| Time Point | Plasmid/Mannoquin 37 Ratios |

| (days) | 0 | 5 | 10 | 20 | 30 |
|---|---|---|---|---|---|
| D 0 | 0.113 | 0.101 | 0.099 | 0.083 | 0.084 |
| D 1 | 0.116 | 0.100 | 0.102 | 0.087 | 0.083 |
| D 2 | 0.119 | 0.105 | 0.105 | 0.086 | 0.086 |
| D 3 | 0.127 | 0.108 | 0.109 | 0.091 | 0.090 |
| D 4 | 0.126 | 0.108 | 0.108 | 0.091 | 0.097 |
| D 7 | 0.128 | 0.110 | 0.111 | 0.094 | 0.100 |
| D 8 | 0.134 | 0.107 | 0.108 | 0.088 | 0.093 |
| D 9 | 0.134 | 0.107 | 0.108 | 0.088 | 0.094 |
| D 10 | 0.137 | 0.109 | 0.112 | 0.091 | 0.095 |
| D 11 | 0.135 | 0.107 | 0.112 | 0.091 | 0.096 |
| D 14 | 0.136 | 0.112 | 0.116 | 0.095 | 0.099 |
| D 15 | 0.136 | 0.111 | 0.116 | 0.095 | 0.092 |
| D 16 | 0.135 | 0.112 | 0.117 | 0.095 | 0.096 |
| D 18 | 0.138 | 0.112 | 0.115 | 0.095 | 0.100 |
| D 21 | 0.137 | 0.113 | 0.117 | 0.097 | 0.099 |
| D 22 | 0.143 | 0.115 | 0.128 | 0.098 | 0.097 |
| D 23 | 0.142 | 0.116 | 0.119 | 0.097 | 0.107 |
| D 24 | 0.142 | 0.119 | 0.117 | 0.100 | 0.106 |
| D 25 | 0.146 | 0.118 | 0.118 | 0.099 | 0.103 |

TABLE 19
Effect of pH on Stability of DNA-Mannoquin 37 Complex at pH 8

| | |
|---|---|
| Time Point | Plasmid/Mannoquin 37 Ratios |

| (days) | 0 | 5 | 10 | 20 | 30 |
|---|---|---|---|---|---|
| D 0 | 0.088 | 0.075 | 0.070 | 0.069 | 0.065 |
| D 1 | 0.087 | 0.075 | 0.069 | 0.070 | 0.066 |
| D 2 | 0.087 | 0.074 | 0.069 | 0.071 | 0.066 |
| D 3 | 0.087 | 0.075 | 0.070 | 0.070 | 0.067 |
| D 4 | 0.088 | 0.076 | 0.071 | 0.070 | 0.068 |
| D 7 | 0.091 | 0.085 | 0.099 | 0.093 | 0.086 |
| D 8 | 0.095 | 0.093 | 0.105 | 0.099 | 0.090 |
| D 9 | 0.098 | 0.092 | 0.103 | 0.097 | 0.085 |
| D 10 | 0.099 | 0.107 | 0.110 | 0.096 | 0.086 |
| D 11 | 0.105 | 0.114 | 0.117 | 0.101 | 0.089 |
| D 14 | 0.088 | 0.106 | 0.095 | 0.095 | 0.086 |
| D 15 | 0.087 | 0.108 | 0.100 | 0.096 | 0.088 |
| D 16 | 0.107 | 0.119 | 0.107 | 0.105 | 0.090 |
| D 18 | 0.097 | 0.110 | 0.095 | 0.095 | 0.091 |
| D 21 | 0.109 | 0.115 | 0.107 | 0.104 | 0.098 |
| D 22 | 0.110 | 0.115 | 0.108 | 0.099 | 0.094 |
| D 23 | 0.112 | 0.111 | 0.106 | 0.095 | 0.085 |
| D 24 | 0.114 | 0.113 | 0.108 | 0.097 | 0.081 |
| D 25 | 0.123 | 0.123 | 0.120 | 0.102 | 0.082 |

TABLE 20
Effect of pHI on Stability of DNA-
Mannoquin 37 Complex at pH 7.4

| | |
|---|---|
| Time Point | Plasmid/Mannoquin 37 Ratios |

| (days) | 0 | 5 | 10 | 20 | 30 |
|---|---|---|---|---|---|
| T0 | 0.114 | 0.089 | 0.082 | 0.078 | 0.077 |
| T6 | 0.106 | 0.095 | 0.091 | 0.077 | 0.076 |
| T7 | 0.106 | 0.094 | 0.091 | 0.078 | 0.076 |
| T8 | 0.106 | 0.094 | 0.091 | 0.077 | 0.076 |
| T9 | 0.101 | 0.099 | 0.089 | 0.077 | 0.076 |
| T12 | 0.100 | 0.098 | 0.091 | 0.077 | 0.075 |
| T13 | 0.101 | 0.102 | 0.089 | 0.079 | 0.075 |
| T14 | 0.102 | 0.104 | 0.088 | 0.078 | 0.075 |

TABLE 21
Effect of pH on Stability of DNA-Mannoquin 38 Complex at pH 2

| | |
|---|---|
| Time Point | Plasmid/Mannoquin 38 Ratios |

| (days) | 0 | 10 | 40 | 50 | 60 |
|---|---|---|---|---|---|
| D 0 | 0.125 | 0.115 | 0.110 | 0.099 | 0.095 |
| D 1 | 0.124 | 0.115 | 0.109 | 0.098 | 0.097 |
| D 2 | 0.124 | 0.114 | 0.109 | 0.099 | 0.098 |
| D 3 | 0.123 | 0.114 | 0.110 | 0.101 | 0.100 |
| D 4 | 0.123 | 0.114 | 0.111 | 0.101 | 0.098 |
| D 7 | 0.125 | 0.115 | 0.116 | 0.102 | 0.101 |
| D 8 | 0.132 | 0.115 | 0.116 | 0.103 | 0.102 |
| D 9 | 0.132 | 0.116 | 0.117 | 0.104 | 0.102 |
| D 10 | 0.136 | 0.116 | 0.117 | 0.104 | 0.102 |
| D 11 | 0.134 | 0.116 | 0.118 | 0.105 | 0.102 |
| D 14 | 0.136 | 0.119 | 0.120 | 0.106 | 0.104 |
| D 15 | 0.138 | 0.118 | 0.120 | 0.118 | 0.104 |
| D 16 | 0.137 | 0.118 | 0.121 | 0.113 | 0.105 |
| D 18 | 0.135 | 0.118 | 0.121 | 0.113 | 0.105 |
| D 21 | 0.131 | 0.115 | 0.122 | 0.113 | 0.107 |
| D 22 | 0.133 | 0.115 | 0.123 | 0.113 | 0.107 |
| D 23 | 0.133 | 0.115 | 0.124 | 0.113 | 0.107 |
| D 24 | 0.137 | 0.115 | 0.125 | 0.109 | 0.108 |
| D 25 | 0.133 | 0.114 | 0.122 | 0.109 | 0.107 |

TABLE 22
Effect of pH on Stability of DNA-Mannoquin 38 Complex at pH 4

| | |
|---|---|
| Time Point | Plasmid/Mannoquin 38 Ratios |

| (days) | 0 | 10 | 40 | 50 | 60 |
|---|---|---|---|---|---|
| D 0 | 0.126 | 0.087 | 0.082 | 0.084 | 0.082 |
| D 1 | 0.123 | 0.089 | 0.081 | 0.084 | 0.083 |
| D 2 | 0.124 | 0.088 | 0.089 | 0.083 | 0.083 |
| D 3 | 0.123 | 0.087 | 0.088 | 0.083 | 0.083 |
| D 4 | 0.124 | 0.087 | 0.089 | 0.083 | 0.083 |
| D 7 | 0.124 | 0.086 | 0.104 | 0.090 | 0.089 |
| D 8 | 0.123 | 0.086 | 0.103 | 0.094 | 0.106 |
| D 9 | 0.123 | 0.086 | 0.101 | 0.103 | 0.125 |
| D 10 | 0.123 | 0.086 | 0.102 | 0.107 | 0.116 |
| D 11 | 0.128 | 0.104 | 0.118 | 0.106 | 0.113 |
| D 14 | 0.125 | 0.086 | 0.105 | 0.104 | 0.110 |
| D 15 | 0.124 | 0.084 | 0.114 | 0.107 | 0.111 |
| D 16 | 0.124 | 0.085 | 0.122 | 0.110 | 0.117 |
| D 18 | 0.121 | 0.085 | 0.117 | 0.108 | 0.114 |
| D 21 | 0.119 | 0.084 | 0.114 | 0.110 | 0.133 |
| D 22 | 0.119 | 0.087 | 0.117 | 0.120 | 0.143 |
| D 23 | 0.118 | 0.084 | 0.122 | 0.145 | 0.149 |
| D 24 | 0.117 | 0.084 | 0.118 | 0.145 | 0.143 |
| D 25 | 0.118 | 0.084 | 0.119 | 0.146 | 0.146 |

TABLE 23
Effect of pH on Stability of DNA-Mannoquin 38 Complex at pH 6

| | |
|---|---|
| Time Point | Plasmid/Mannoquin 38 Ratios |

| (days) | 0 | 10 | 40 | 50 | 60 |
|---|---|---|---|---|---|
| D 0 | 0.119 | 0.106 | 0.080 | 0.082 | 0.102 |
| D 1 | 0.124 | 0.104 | 0.084 | 0.086 | 0.102 |
| D 2 | 0.131 | 0.112 | 0.087 | 0.086 | 0.104 |
| D 3 | 0.119 | 0.113 | 0.090 | 0.090 | 0.109 |
| D 4 | 0.118 | 0.112 | 0.090 | 0.091 | 0.110 |
| D 7 | 0.119 | 0.115 | 0.091 | 0.093 | 0.111 |
| D 8 | 0.127 | 0.113 | 0.093 | 0.091 | 0.109 |
| D 9 | 0.135 | 0.112 | 0.093 | 0.095 | 0.114 |
| D 10 | 0.134 | 0.113 | 0.093 | 0.095 | 0.114 |
| D 11 | 0.134 | 0.113 | 0.093 | 0.095 | 0.113 |
| D 14 | 0.133 | 0.116 | 0.095 | 0.096 | 0.115 |
| D 15 | 0.134 | 0.115 | 0.094 | 0.095 | 0.117 |
| D 16 | 0.134 | 0.117 | 0.094 | 0.096 | 0.114 |
| D 18 | 0.135 | 0.116 | 0.091 | 0.093 | 0.115 |
| D 21 | 0.142 | 0.118 | 0.092 | 0.094 | 0.120 |
| D 22 | 0.140 | 0.119 | 0.096 | 0.093 | 0.115 |
| D 23 | 0.139 | 0.120 | 0.096 | 0.093 | 0.114 |
| D 24 | 0.137 | 0.117 | 0.098 | 0.093 | 0.113 |
| D 25 | 0.140 | 0.119 | 0.098 | 0.094 | 0.113 |

TABLE 24
Effect of pH on Stability of DNA-Mannoquin 38 Complex at pH 8

| | |
|---|---|
| Time Point | Plasmid/Mannoquin 38 Ratios |

| (days) | 0 | 10 | 40 | 50 | 60 |
|---|---|---|---|---|---|
| D 0 | 0.114 | 0.086 | 0.077 | 0.080 | 0.082 |
| D 1 | 0.114 | 0.085 | 0.091 | 0.081 | 0.081 |
| D 2 | 0.113 | 0.084 | 0.079 | 0.081 | 0.081 |
| D 3 | 0.113 | 0.085 | 0.081 | 0.082 | 0.081 |
| D 4 | 0.114 | 0.087 | 0.082 | 0.083 | 0.081 |
| D 7 | 0.116 | 0.096 | 0.087 | 0.104 | 0.095 |
| D 8 | 0.115 | 0.099 | 0.095 | 0.110 | 0.107 |
| D 9 | 0.116 | 0.101 | 0.103 | 0.111 | 0.102 |
| D 10 | 0.120 | 0.114 | 0.104 | 0.123 | 0.099 |
| D 11 | 0.121 | 0.119 | 0.106 | 0.117 | 0.102 |
| D 14 | 0.125 | 0.115 | 0.101 | 0.116 | 0.103 |
| D 15 | 0.126 | 0.119 | 0.104 | 0.117 | 0.103 |
| D 16 | 0.135 | 0.133 | 0.108 | 0.122 | 0.104 |
| D 18 | 0.129 | 0.120 | 0.105 | 0.107 | 0.102 |
| D 21 | 0.136 | 0.132 | 0.116 | 0.120 | 0.111 |
| D 22 | 0.151 | 0.128 | 0.114 | 0.121 | 0.114 |
| D 23 | 0.146 | 0.124 | 0.121 | 0.120 | 0.113 |
| D 24 | 0.142 | 0.126 | 0.111 | 0.123 | 0.107 |
| D 25 | 0.151 | 0.134 | 0.116 | 0.124 | 0.117 |

TABLE 25
Effect of pH on Stability of DNA-
Mannoquin 38 Complex at pH 7.4

| | |
|---|---|
| Time Point | Plasmid/Mannoquin 38 Ratios |

| (days) | 0 | 10 | 40 | 50 | 60 |
|---|---|---|---|---|---|
| T0 | 0.121 | 0.090 | 0.082 | 0.084 | 0.082 |
| T6 | 0.116 | 0.088 | 0.084 | 0.087 | 0.084 |
| T7 | 0.116 | 0.088 | 0.087 | 0.087 | 0.083 |
| T8 | 0.116 | 0.088 | 0.089 | 0.087 | 0.083 |
| T9 | 0.108 | 0.086 | 0.089 | 0.086 | 0.082 |
| T12 | 0.110 | 0.087 | 0.089 | 0.085 | 0.083 |
| T13 | 0.111 | 0.086 | 0.092 | 0.085 | 0.083 |
| T14 | 0.110 | 0.086 | 0.092 | 0.086 | 0.084 |

TABLE 26
Effect of pH on Stability of DNA-Mannoquin 39 Complex at pH 2

| | |
|---|---|
| Time Point | Plasmid/Mannoquin 39 Ratios |

| (days) | 0 | 5 | 30 | 40 | 50 |
|---|---|---|---|---|---|
| D 0 | 0.119 | 0.119 | 0.108 | 0.106 | 0.100 |
| D 1 | 0.119 | 0.115 | 0.108 | 0.108 | 0.099 |
| D 2 | 0.118 | 0.114 | 0.109 | 0.108 | 0.100 |
| D 3 | 0.118 | 0.114 | 0.110 | 0.109 | 0.101 |
| D 4 | 0.118 | 0.114 | 0.110 | 0.108 | 0.101 |
| D 7 | 0.117 | 0.114 | 0.111 | 0.110 | 0.103 |
| D 8 | 0.118 | 0.114 | 0.114 | 0.110 | 0.103 |
| D 9 | 0.122 | 0.114 | 0.113 | 0.110 | 0.103 |
| D 10 | 0.126 | 0.114 | 0.112 | 0.110 | 0.103 |
| D 11 | 0.125 | 0.114 | 0.112 | 0.110 | 0.104 |
| D 14 | 0.123 | 0.114 | 0.112 | 0.112 | 0.110 |
| D 15 | 0.124 | 0.114 | 0.111 | 0.111 | 0.108 |
| D 16 | 0.122 | 0.114 | 0.112 | 0.112 | 0.106 |
| D 18 | 0.124 | 0.113 | 0.113 | 0.112 | 0.107 |
| D 21 | 0.116 | 0.113 | 0.113 | 0.113 | 0.107 |
| D 22 | 0.115 | 0.115 | 0.112 | 0.114 | 0.108 |
| D 23 | 0.117 | 0.115 | 0.112 | 0.114 | 0.109 |
| D 24 | 0.114 | 0.114 | 0.111 | 0.114 | 0.109 |
| D 25 | 0.115 | 0.114 | 0.112 | 0.114 | 0.109 |

TABLE 27
Effect of pH on Stability of DNA-Mannoquin 38 Complex at pH 4

| | |
|---|---|
| Time Point | Plasmid/Mannoquin 38 Ratios |

| (days) | 0 | 5 | 30 | 40 | 50 |
|---|---|---|---|---|---|
| D 0 | 0.117 | 0.096 | 0.079 | 0.079 | 0.079 |
| D 1 | 0.118 | 0.095 | 0.079 | 0.079 | 0.080 |
| D 2 | 0.117 | 0.094 | 0.078 | 0.079 | 0.080 |
| D 3 | 0.117 | 0.093 | 0.079 | 0.079 | 0.080 |
| D 4 | 0.117 | 0.093 | 0.079 | 0.079 | 0.080 |
| D 7 | 0.114 | 0.092 | 0.095 | 0.100 | 0.090 |
| D 8 | 0.113 | 0.093 | 0.104 | 0.105 | 0.099 |
| D 9 | 0.112 | 0.093 | 0.104 | 0.106 | 0.102 |
| D 10 | 0.110 | 0.093 | 0.108 | 0.108 | 0.101 |
| D 11 | 0.108 | 0.093 | 0.110 | 0.110 | 0.102 |
| D 14 | 0.099 | 0.093 | 0.116 | 0.111 | 0.103 |
| D 15 | 0.094 | 0.092 | 0.119 | 0.115 | 0.104 |
| D 16 | 0.077 | 0.093 | 0.126 | 0.120 | 0.110 |
| D 18 | 0.058 | 0.092 | 0.141 | 0.119 | 0.111 |
| D 21 | 0.058 | 0.092 | 0.126 | 0.122 | 0.115 |
| D 22 | 0.058 | 0.090 | 0.121 | 0.131 | 0.118 |
| D 23 | 0.058 | 0.092 | 0.122 | 0.129 | 0.120 |
| D 24 | 0.058 | 0.091 | 0.122 | 0.125 | 0.129 |
| D 25 | 0.058 | 0.090 | 0.127 | 0.128 | 0.118 |

TABLE 28
Effect of pH on Stability of DNA-Mannoquin 38 Complex at pH 6

| | |
|---|---|
| Time Point | Plasmid/Mannoquin 38 Ratios |

| (days) | 0 | 5 | 30 | 40 | 50 |
|---|---|---|---|---|---|
| D 0 | 0.117 | 0.108 | 0.081 | 0.086 | 0.081 |
| D 1 | 0.120 | 0.105 | 0.080 | 0.090 | 0.084 |
| D 2 | 0.118 | 0.110 | 0.085 | 0.088 | 0.085 |
| D 3 | 0.118 | 0.111 | 0.086 | 0.092 | 0.085 |
| D 4 | 0.121 | 0.111 | 0.086 | 0.092 | 0.086 |
| D 7 | 0.122 | 0.116 | 0.088 | 0.095 | 0.088 |
| D 8 | 0.126 | 0.116 | 0.087 | 0.093 | 0.087 |
| D 9 | 0.131 | 0.116 | 0.086 | 0.091 | 0.086 |
| D 10 | 0.130 | 0.117 | 0.087 | 0.093 | 0.087 |
| D 11 | 0.127 | 0.118 | 0.088 | 0.094 | 0.091 |
| D 14 | 0.133 | 0.120 | 0.089 | 0.096 | 0.092 |
| D 15 | 0.133 | 0.118 | 0.088 | 0.096 | 0.092 |
| D 16 | 0.139 | 0.124 | 0.088 | 0.096 | 0.089 |
| D 18 | 0.140 | 0.122 | 0.087 | 0.095 | 0.090 |
| D 21 | 0.141 | 0.123 | 0.088 | 0.110 | 0.092 |
| D 22 | 0.139 | 0.125 | 0.093 | 0.104 | 0.091 |
| D 23 | 0.138 | 0.125 | 0.095 | 0.104 | 0.091 |
| D 24 | 0.137 | 0.124 | 0.097 | 0.102 | 0.090 |
| D 25 | 0.139 | 0.123 | 0.098 | 0.105 | 0.090 |

TABLE 29
Effect of pH on Stability of DNA-Mannoquin 38 Complex at pH 8

| | |
|---|---|
| Time Point | Plasmid/Mannoquin 38 Ratios |

| (days) | 0 | 5 | 30 | 40 | 50 |
|---|---|---|---|---|---|
| D 0 | 0.106 | 0.091 | | 0.077 | 0.080 |
| D 1 | 0.106 | 0.089 | 0.082 | 0.077 | 0.076 |
| D 2 | 0.105 | 0.088 | 0.074 | 0.077 | 0.076 |
| D 3 | 0.105 | 0.089 | 0.073 | 0.080 | 0.076 |
| D 4 | 0.105 | 0.089 | 0.076 | 0.079 | 0.078 |
| D 7 | 0.109 | 0.094 | 0.068 | 0.090 | 0.085 |
| D 8 | 0.113 | 0.095 | 0.076 | 0.118 | 0.096 |
| D 9 | 0.116 | 0.099 | 0.083 | 0.111 | 0.101 |
| D 10 | 0.119 | 0.101 | 0.093 | 0.104 | 0.097 |
| D 11 | 0.126 | 0.103 | 0.145 | 0.108 | 0.101 |
| D 14 | 0.128 | 0.106 | 0.128 | 0.101 | 0.100 |
| D 15 | 0.143 | 0.107 | 0.183 | 0.101 | 0.100 |
| D 16 | 0.144 | 0.120 | 0.207 | 0.113 | 0.104 |
| D 18 | 0.154 | 0.118 | 0.229 | 0.106 | 0.102 |
| D 21 | 0.124 | 0.120 | 0.204 | 0.149 | 0.229 |
| D 22 | 0.121 | 0.124 | 0.186 | 0.142 | 0.137 |
| D 23 | 0.120 | 0.123 | 0.070 | 0.136 | 0.118 |
| D 24 | 0.135 | 0.141 | 0.052 | 0.135 | 0.122 |
| D 25 | 0.151 | 0.152 | 0.050 | 0.143 | 0.128 |

TABLE 30
Effect of pH on Stability of DNA-
Mannoquin 39 Complex at pH 7.4

| | |
|---|---|
| Time Point | Plasmid/Mannoquin 39 Ratios |

| (days) | 0 | 5 | 30 | 40 | 50 |
|---|---|---|---|---|---|
| T0 | 0.111 | 0.091 | 0.078 | 0.079 | 0.079 |
| T6 | 0.107 | 0.087 | 0.078 | 0.083 | 0.080 |
| T7 | 0.107 | 0.087 | 0.078 | 0.083 | 0.080 |
| T8 | 0.107 | 0.087 | 0.078 | 0.082 | 0.079 |
| T9 | 0.101 | 0.083 | 0.077 | 0.083 | 0.079 |
| T12 | 0.101 | 0.083 | 0.077 | 0.085 | 0.078 |
| T13 | 0.101 | 0.083 | 0.077 | 0.085 | 0.079 |
| T14 | 0.101 | 0.082 | 0.077 | 0.085 | 0.078 |

### Example 9: Control Data for Mannoquin Stability at Different pH

The stability of mannoquin over time (day 1 to 11) and at varying pH (2, 4, 6, and 8) was studied. The reaction volume was 200 μL, the mannoquin concentration was approximately 30 μM (0.1 mg/mL), and no DNA was added, PBS with HCl or NaOH was used to adjust the pH, Absorption spectra from 340 to 550 nm were collected on the indicated days, Results, in the form of absorbance at 424 nm, appear in Tables 30 to 37,

TABLE 30
Effect of pH on Stability of Mannoquin 36

| | |
|---|---|
| Time Point | pH in PBS Buffer |

| (days) | 2 | 4 | 6 | 8 |
|---|---|---|---|---|
| D 0 | 0.124 | 0.121 | 0.113 | 0.088 |
| D 1 | 0.123 | 0.125 | 0.114 | 0.087 |
| D 2 | 0.145 | 0.124 | 0.117 | 0.087 |
| D 3 | 0.139 | 0.123 | 0.127 | 0.087 |
| D 4 | 0.121 | 0.129 | 0.126 | 0.088 |
| D 7 | 0.119 | 0.128 | 0.126 | 0.104 |
| D 8 | 0.118 | 0.127 | 0.133 | 0.108 |
| D 9 | 0.122 | 0.132 | 0.135 | 0.108 |
| D 10 | 0.122 | 0.121 | 0.133 | 0.118 |
| D 11 | 0.124 | 0.119 | 0.136 | 0.115 |

TABLE 31
Stability of Mannoquin 36 at pH 7.4

| | pH in PBS Buffer |
|---|---|
| Time Point (days) | 7.4 |
| D 0 | 0.126 |
| D 1 | 0.118 |
| D 2 | 0.118 |
| D 5 | 0.118 |
| D 6 | 0.118 |
| D 7 | 0.117 |
| D 8 | 0.117 |
| D 9 | 0.108 |
| D 12 | 0.112 |

TABLE 32
Effect of pH on Stability of Mannoquin 37

| | |
|---|---|
| Time Point | pH in PBS Buffer |

| (days) | 2 | 4 | 6 | 8 |
|---|---|---|---|---|
| D 0 | 0.120 | 0.113 | 0.113 | 0.088 |
| D 1 | 0.119 | 0.111 | 0.116 | 0.087 |
| D 2 | 0.119 | 0.110 | 0.119 | 0.087 |
| D 3 | 0.119 | 0.109 | 0.127 | 0.087 |
| D 4 | 0.118 | 0.116 | 0.126 | 0.088 |
| D 7 | 0.119 | 0.111 | 0.128 | 0.091 |
| D 8 | 0.118 | 0.111 | 0.134 | 0.095 |
| D 9 | 0.118 | 0.111 | 0.134 | 0.098 |
| D 10 | 0.118 | 0.111 | 0.137 | 0.099 |
| D 11 | 0.118 | 0.111 | 0.135 | 0.105 |

TABLE 33
Stability of Mannoquin 37 at pH 7.4

| Time Point (days) | Absorbance at pH 7.4 |
|---|---|
| D 0 | 0.114 |
| D 1 | 0.107 |
| D 2 | 0.106 |
| D 5 | 0.106 |
| D 6 | 0.106 |
| D 7 | 0.106 |
| D 8 | 0.106 |
| D 9 | 0.101 |
| D 12 | 0.100 |

TABLE 34
Effect of pH on Stability of Mannoquin 38

| | |
|---|---|
| Time Point | pH in PBS Buffer |

| (days) | 2 | 4 | 6 | 8 |
|---|---|---|---|---|
| D 0 | 0.125 | 0.126 | 0.119 | 0.114 |
| D 1 | 0.124 | 0.123 | 0.124 | 0.114 |
| D 2 | 0.124 | 0.124 | 0.131 | 0.113 |
| D 3 | 0.123 | 0.123 | 0.119 | 0.113 |
| D 4 | 0.123 | 0.124 | 0.118 | 0.114 |
| D 7 | 0.125 | 0.124 | 0.119 | 0.116 |
| D 8 | 0.132 | 0.123 | 0.127 | 0.115 |
| D 9 | 0.132 | 0.123 | 0.135 | 0.116 |
| D 10 | 0.136 | 0.123 | 0.134 | 0.120 |
| D 11 | 0.134 | 0.128 | 0.134 | 0.121 |

TABLE 35
Stability of Mannoquin 38 at pH 7.4

| | pH in PBS Buffer |
|---|---|
| Time Point (days) | 7.4 |
| D 0 | 0.121 |
| D 1 | 0.117 |
| D 2 | 0.117 |
| D 5 | 0.116 |
| D 6 | 0.116 |
| D 7 | 0.116 |
| D 8 | 0.116 |
| D 9 | 0.108 |
| D 12 | 0.110 |

TABLE 36
Effect of pH on Stability of Mannoquin 39

| | |
|---|---|
| Time Point | pH in PBS Buffer |

| (days) | 2 | 4 | 6 | 8 |
|---|---|---|---|---|
| D 0 | 0.119 | 0.117 | 0.117 | 0.106 |
| D 1 | 0.119 | 0.118 | 0.120 | 0.106 |
| D 2 | 0.118 | 0.117 | 0.118 | 0.105 |
| D 3 | 0.118 | 0.117 | 0.118 | 0.105 |
| D 4 | 0.118 | 0.117 | 0.121 | 0.105 |
| D 7 | 0.117 | 0.114 | 0.122 | 0.109 |
| D 8 | 0.118 | 0.113 | 0.126 | 0.113 |
| D 9 | 0.122 | 0.112 | 0.131 | 0.116 |
| D 10 | 0.126 | 0.110 | 0.130 | 0.119 |
| D 11 | 0.125 | 0.108 | 0.127 | 0.126 |

TABLE 37
Stability of Mannoquin 39 at pH 7.4

| | pH in PBS Buffer |
|---|---|
| Time Point (days) | 7.4 |
| D 0 | 0.111 |
| D 1 | 0.108 |
| D 2 | 0.108 |
| D 5 | 0.107 |
| D 6 | 0.107 |
| D 7 | 0.107 |
| D 8 | 0.107 |
| D 9 | 0.101 |
| D 12 | 0.101 |

### Example 10: Cellular Delivery

The efficacy of mannoquin (MQ) compound, Mannoquin 38 (SY21020276-1, also referred to as MQ-3943) for the delivery of IL-10 plasmid DNA (pDNA) into selected immune cells was studied, Cells were supplemented with D-mannose, Mannoquin 38, and pDNA, in this example, XT-150, under different conditions. The cell line used was NR8383 rat alveolar macrophages, which grow as a mixture of suspended and adherent cells. The culture medium was F-12K Medium+15% FBS. The cells were plated in a 24-well format at a seeding density of 4-6×10⁵ cells/mL into the wells of a 24-well plate. All conditions were performed in duplicate. The testing conditions used NR8383 cells in both unactivated and activated states. Activation was achieved by the addition of 1 μg/mL of lipopolysaccharides (LPS) into the growth medium of the appropriate wells for at least 16 hours prior to subsequent manipulations. Activated macrophages are characteristic of macrophages found in inflammatory disease states.

The test agents being examined for their ability to enhance uptake of the IL-10 pDNA were D-mannose and the MQ compound (e.g., SY21020276-1/Mannoquin 38). Either 0, 1, or 2 μg pDNA was used per well, with or without mannose. The combination of pDNA minus D-mannose serves as pDNA control for all conditions. The combination of pDNA with uncomplexed Mannoquin 38 was completed by adding separate pDNA and Mannoquin 38 solutions to culture. Also, pDNA with complexed Mannoquin 38 were studied wherein pre-incubated pDNA and Mannoquin 38 solutions were added to the culture.

Lipofectamine 2000 transfections per manufacturer's instructions was used as a positive control. Separate confirmation of successful transfection with Lipofectamine 2000 has been confirmed.

The pDNA solution used was 40 μg/mL pDNA in PBS (herein after “Working pDNA solution”).

The Test Agent Working solutions were:
Working D-mannose solution which comprised 1 μg/mL D-mannose in PBS (herein after “Working D-mannose solution”)
Working Mannoquin 38 solution which comprised 1 μg/mL Mannoquin 38 in PBS (herein after “Mannoquin 38 solution”).

For studies with no pDNA, 100 μL of 1×PBS was added to the appropriate culture wells.

For plasmid-only conditions, to the appropriate culture wells, 100 μL of DNA solutions was added, wherein the solution was 50 μL of Working pDNA solution+150 μL of 1×PBS (1 μg pDNA) or 100 μL of Working pDNA solution+100 μL of 1×PBS (2 μg pDNA).

For non-complexed test agent conditions, to the appropriate culture wells, 50 μL of each separately prepared solution was added: DNA solution (0, 25, or 50 μL Working pDNA solution+100, 75, or 50 μL 1×PBS, respectively) or test agent solution (25, 25, or 50 μL Test Agent Working solution+75, 75, or 50 μL 1×PBS, respectively).

For complexed test agent conditions, to the appropriate culture wells, 100 μL of each of three combined solutions was added: 0, 50, or 100 μL Working pDNA solution+50, 50, or 100 μL Test Agent Working solution+150, 100, or 0 μL 1×PBS, respectively.

All solutions are prepared and incubated at ambient temperature for 30 minutes prior to addition to cultures. After addition of the respective transfection reagents, the cells are incubated at 37° C. in a 5% CO₂ for about 24 hours. At the end of the cell incubation, the medium containing suspended cells was collected from each well (500 μL) into a 1.5 mL tube and stored frozen. The adherent cells were washed with PBS and then removed from the plate by trypsinization and resuspended in culture medium. The trypsinized cells were transferred to a second 1.5 mL tube for the respective culture well. The tubes were centrifuged with the original supernatant and the trypsinized cells for 5 minutes at 300 rcf. The original supernatant was collected to a new 1.5 mL tube. All cell pellets were resuspended, washed twice with 1×PBS+0.1% BSA, and centrifuged for 5 minutes at 300 ref. The cells were resuspended in 75 μL of PBS+0.1% BSA, counted with Trypan Blue solution, and the suspension was diluted to 4×10⁶ cells/mL (4,000 cells/μL) using 1x PBS+0.1% BSA. 100 μM stocks of the PCR primers and probe below were prepared. PCR samples using 500-900 nM of each of the primers and 250 nM probe per reaction were prepared.

CMV Forward Primer:
5′-CGTTACATAACTTACGGTAAATGG-3′
CMV Reverse Primer:
5′-CCCTATTGGCGTTACTATGG-3′
CMV TaqMan® Probe:
5′-CTGACCGCCCAACGACCC-3′

The 1×ddPCR master mix was prepared with all components according to the manufacturer's protocol. The 1×ddPCR master mix was mixed by vortexing. The 4,000 cells/μL cell suspension were vortexed to mix immediately before adding to the 1×ddPCR master mix. Up to 5 μL of cell suspension was added to 20 μL of the 1×ddPCR master mix and diluted to a final reaction volume of 25 μL using nuclease-free water. After adding the cells, the reaction solution was immediately vortexed to mix. 20 μL of the ddPCR reaction solution was added to the appropriate wells of the droplet generator tray. PCR reaction was performed and read according to the manufacturer's protocol.

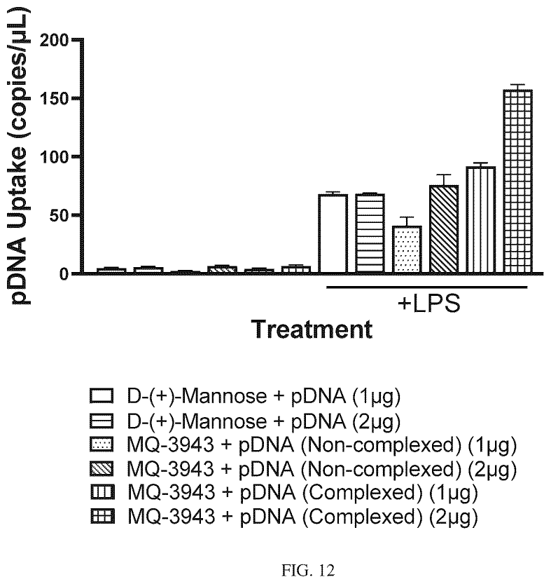

The results appear in FIG. 12 and a summary of the solutions used in the procedure appears below in Table 38.

TABLE 38
Solution Identity for Example 10 and FIG. 12

| | | | | Mannoquin | Mannoquin | Mannoquin | Mannoquin |
|---|---|---|---|---|---|---|---|
| | | D- | D- | 38 + | 38 + | 38 + | 38 + |
| | | Mannose + | Mannose + | pDNA | pDNA | pDNA | pDNA |
| | | pDNA | pDNA | 1 ug (non- | 2 ug (non- | 1 ug | 2 ug |
| | | 1 ug | 2 ug | complexed) | complexed) | (complexed) | (complexed) |
| Working | 1 μg/mL D- | 37.5 μL | 75 μL | X | X | X | X |
| D- | mannose | | | | | | |
| mannose | in PBS | | | | | | |
| solution | | | | | | | |
| Working | 0.67 μg/mL | X | X | 37.5 μL | 75 μL | 37.5 μL | 75 μL |
| Mannoquin | Mannoquin | | | | | | |
| 38 solution | 38 in PBS | | | | | | |
| Working | 40 μg/mL | 37.5 μL | 75 μL | 37.5 μL | 75 μL | 37.5 μL | 75 μL |
| pDNA | pDNA in | | | | | | |
| solution | PBS | | | | | | |

| | | | | | | |
|---|---|---|---|---|---|---|
| PBS diluent for | 112.5 μL/ | 75 μL/ | 112.5 μL/ | 75 μL/ | 225 μL | 150 μL |
| compound/PBS | 112.5 μL | 75 μL | 112.5 μL | 75 μL | | |
| diluent for pDNA | | | | | | |
| total volume of | 150 μL/ | 150 μL/ | 150 μL/ | 150 μL/ | 300 μL | 300 μL |
| transfection | 150 μL | 150 μL | 150 μL | 150 μL | | |
| solution(s) | | | | | | |
| total transfection | 50 μL/ | 50 μL/ | 50 μL/ | 50 μL/ | 100 μL | 100 μL |
| solution(s) added | 50 μL | 50 μL | 50 μL | 50 μL | | |
| to well | | | | | | |

Plasmid DNA uptake was confirmed for cells treated with LPS (i.e., activated NR8383 rat alveolar macrophages). For D-mannose plus pDNA-treated cells, 68 and 68.4 pDNA copies per μL cell suspension were observed at 1 and 2 μg pDNA incubation, respectively. Thus, there was no dose-dependency of uptake based on pDNA level under these conditions.

For non-complexed Mannoquin 38 and pDNA-treated cells, 41.2 and 75.9 pDNA copies per μL cell suspension were observed at 1 and 2 μg pDNA incubation, respectively. Thus, there was a dose-dependency of uptake based on pDNA level under these conditions, a 1.84-fold increase similar to the 2-fold difference in pDNA content.

For complexed Mannoquin 38:pDNA-treated cells, 91.8 and 157.7 pDNA copies per μL cell suspension were observed at 1 and 2 μg pDNA incubation, respectively. Thus, there was a dose-dependency of uptake based on pDNA level under these conditions. At 1 μg pDNA incubation with the complex, the individual values for pDNA per unit volume were higher than any of the individual values at the 2 μg pDNA incubation with non-complexed Mannoquin 38. At 2 Mg pDNA incubation with the complex, the individual values for pDNA per unit volume were higher than any of the individual values at the 1 μg pDNA incubation with complexed Mannoquin 38.

Based on these results, the complexation of Mannoquin 38 and pDNA led to dose-dependent increases in pDNA uptake by LPS-activated NR8383 greater than those observed at the same pDNA doses with either D-mannose or non-complexed Mannoquin 38. Thus, the Mannoquin 38:pDNA complex represents an improved cellular uptake vehicle for pDNA.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

1. A compound of Formula (I): 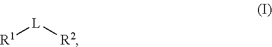
or a pharmaceutically acceptable salt thereof, wherein:
R¹ is substituted or unsubstituted sugar, substituted or unsubstituted amino sugar, or substituted or unsubstituted deoxy sugar;
L is substituted C₁₋₂₀ alkylene or unsubstituted C₁₋₂₀ alkylene, wherein:
optionally one or more backbone carbon atoms in the substituted C₁₋₂₀ alkylene or unsubstituted C₁₋₂₀ alkylene are independently replaced with —NR^{A}—, —O—, —C(═O)—, —S—, substituted or unsubstituted carbocyclylene, substituted or unsubstituted heterocyclylene, substituted or unsubstituted arylene, or substituted or unsubstituted heteroarylene;
R² is a DNA-interacting agent; and
R^{A} is hydrogen, substituted or unsubstituted C₁₋₆ alkyl, —C(═O)(C₁₋₆ alkyl), or a nitrogen protecting group;
provided that the compound is not of the formula:

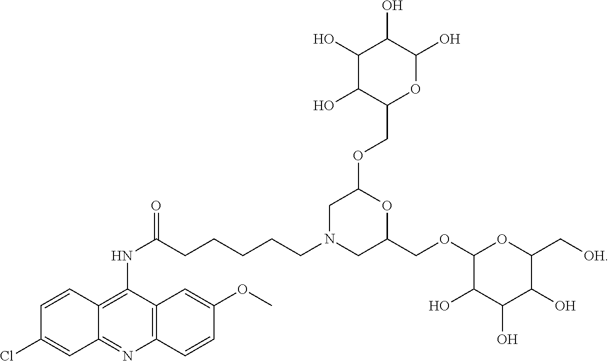 [SMILES: COc1ccc2nc3cc(Cl)ccc3c(NC(=O)CCCCCN3CC(COC4OC(CO)C(O)C(O)C4O)OC(OCC4OC(O)C(O)C(O)C4O)C3)c2c1]

2. (canceled)

3. The compound of claim 1, wherein R² is selected from the group consisting of agent is selected from the group consisting of acridine (e.g., 6-chloro-2-methoxyacridine, 6-chloro-2-methoxyacridin-9-amine), actinomycins, acriflavine, amsacrine, amonafide, anthracyclines, berberine, benzophenone, daunomycin, dihydroethidium, dimidium, doxorubicin, ethidium, lucanthone, hycanthone, naphthalene tetra carboxylic diimides, nogalomycin, oxazolopyridocarbozole, phenanthrene, 1,10-phenathroline, phenanthridine, proflavine, propidium, pyrazoloacridine, quinacrine, thalidomide, thioxanthone, tilorone, and other organic molecules comprising a planar aromatic or heteroaromatic moiety capable of stacking between the nucleobases of DNA.

4-5. (canceled)

6. The compound of claim 1, wherein the compound is of Formula (I-A): 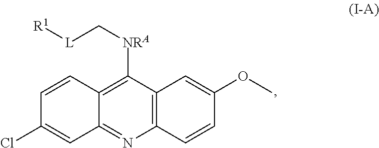
or a pharmaceutically acceptable salt thereof.

7-15. (canceled)

16. The compound of claim 1, wherein R¹ is allose, altose, arabinose, dextrose, erythrose, erythrulose, fructose, galactose, glucose, gulose, idose, lactose, lyxose, maltose, mannose, psicose, ribose, ribulose, sorbose, sucrose, tagatose, talose, threose, or xylose, or derivative thereof; N-acetylglucosamine; N-acetylgalactosamine; N-acetylmannosamine; glucuronic acid; chondroitin sulfate; hyaluronic acid; fucose; fuculose; or rhamnose.

17-20. (canceled)

21. The compound of claim 1, wherein R¹ is selected from the group consisting of: 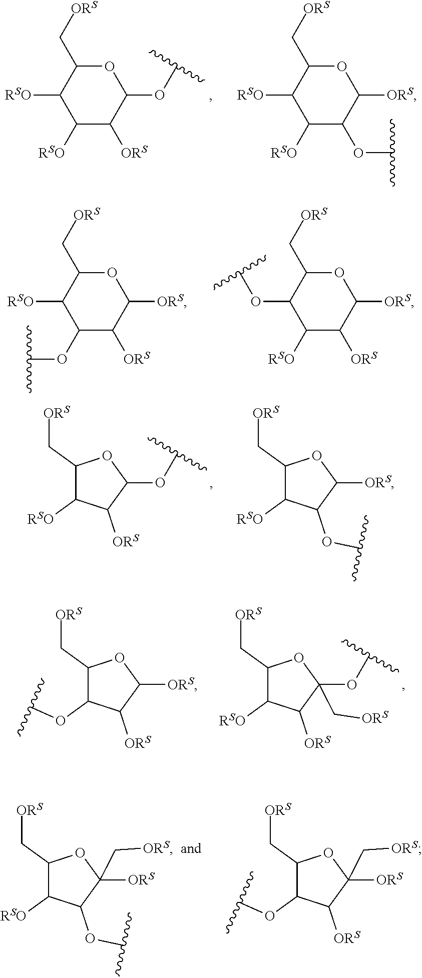
wherein R^{S} is selected from the group consisting of hydrogen, C₁₋₆ alkyl, —C(═O)(C₁₋₆ alkyl), an oxygen protecting group, —S(═O)₂(OH), —S(═O)₂(OH)₂, and —P(═O)(OH)₂, and salts thereof.

22-24. (canceled)

25. The compound of claim 1, wherein the sugar is selected from: 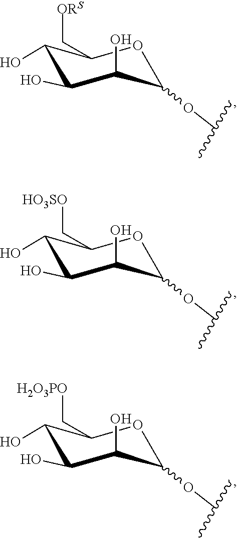
or salt thereof.

26. The compound of claim 1, wherein the compound is of the formula: 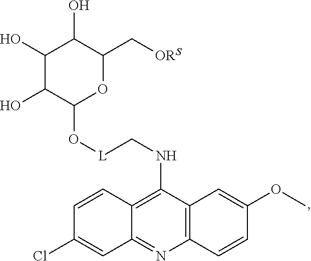
or a pharmaceutically acceptable salt thereof.

27. The compound of claim 1, wherein the compound is of the formula: 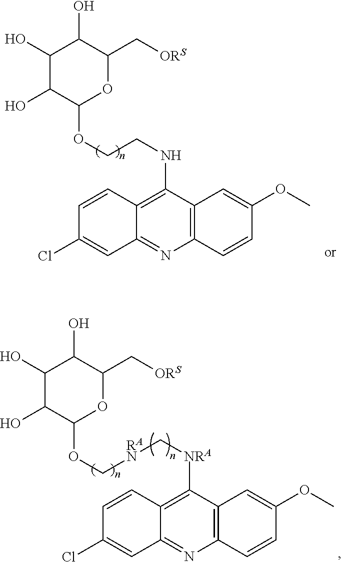
or a pharmaceutically acceptable salt thereof, wherein each instance of n is independently an integer from 1-8, inclusive.

28. The compound of claim 1, wherein the compound is of the formula: 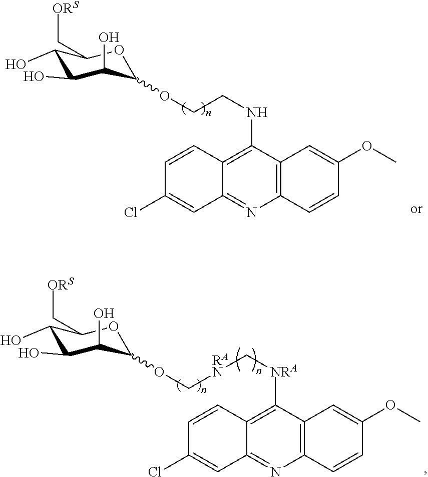
or a pharmaceutically acceptable salt thereof, wherein each instance of n is independently an integer from 1-8, inclusive.

29. A composition comprising the compound of claim 1 and a pharmaceutically acceptable excipient.

30. A composition comprising the compound of claim 1 and DNA.

31. A composition comprising the compound of claim 1 and a plasmid.

32. The composition of claim 31, wherein the plasmid encodes a IL-10 polypeptide.

33. The composition of claim 32, wherein the IL-10 polypeptide comprises a mutation.

34. The composition of claim 33, wherein the mutation is within the portion of the IL-10 polypeptide comprising: LRRCHRFLPCENKSK (SEQ ID NO: 26).

35-54. (canceled)

55. A method of delivering a compound of claim 1 to a subject, the method comprising administering an effective amount of the compound to a subject in need thereof.

56. A method of delivering DNA to a subject, the method comprising administering an effective amount of a composition of claim 30 to a subject in need thereof.

57-58. (canceled)

59. A method of delivering a plasmid to a subject, the method comprising administering an effective amount of a composition of claim 31 to a subject in need thereof.

60. A method of treating or preventing a disease or disorder in a subject, the method comprising administering an effective amount of a composition of claim 31 to a subject in need thereof.

61-78. (canceled)

79. A kit comprising:
the compound of claim 1; and
and instructions for administering to a subject the compound or composition.


SEQUENCE LISTING (29 sequences)

SEQ ID NO: 1 - protein, 178 aa, synthetic construct
  REGION 1..178: note = Synthetic
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRN

SEQ ID NO: 2 - DNA, 5899 bp, synthetic construct
  misc_feature 1..5899: note = Synthetic
  source 1..5899: mol_type = other DNA
agctttgcagccgggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtaattgaaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtaaccgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagggctgcagcatgcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtatcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgatatcaagcttggtaccgagctcggatccactagtaacggccgccagtgtgctggaattcgcccttccgcagcgccagcatgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatctcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgagacatcagggtggcaagccgaattccccctagcggaagggcgaattctgcagaatcggatccatcgataccgtcgacctcgagggggggcccggtacccaattcgccctatagtgagtcgtattacgcgcgcagcggccgaccatggcccaacttgtttattgcagcttataatggttacaaataaagcaatagcatcacaaatttcacaaataaagcatttttttcactgcattctagttgtggtttgtccaaactcatcaatgtatcttatcatgtctggatctccggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggtctgagacaataaccctgataaatgcttcaataatgta

SEQ ID NO: 3 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 4 - protein, 178 aa, Rattus norvegicus
MPGSALLCCLLLLAGVKTSKGHSIRGDNNCTHFPVSQTHMLRELRAAFSQVKTFFQKKDQLDNILLTDSLLQDFKGYLGCQALSEMIKFYLVEVMPQAENHGPEIKEHLNSLGEKLKTLWIQLRRCHRFLPCENKSKAVEQVKNDFNKLQDKGVYKAMNEFDIFINCIEAYVTLKMKN

SEQ ID NO: 5 - protein, 178 aa, Rattus norvegicus
MLGSALLCCLLLLAGVKTSKGHSIRGDNNCTHFPVSQTHMLRELRAAFSQVKTFFQKKDQLDNIVLTDSLLQDFKGYLGCQALSEMIKFYLVEVMPQAENHGPEIKEHLNSLGEKLKTLWIQLRRCHRFLPCENKSKAVEQVKNDFNKLQDKGVYKAMNEFDIFINCIEAYVTLKMKN

SEQ ID NO: 6 - protein, 178 aa, Mus musculus
MPGSALLCCLLLLTGMRISRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 7 - protein, 178 aa, Equus caballus
MHSSALLCYLVFLAGVGASRDRGTQSENSCTHFPTSLPHMLHELRAAFSRVKTFFQMKDQLDNMLLNGSLLEDFKGYLGCQALSEMIQFYLEEVMPQAENHGPDIKEHVNSLGEKLKTLRVRLRRCHRFLPCENKSKAVEQVKSAFSKLQEKGVYKAMSEFDIFINYIEAYMTTKMKN

SEQ ID NO: 8 - protein, 178 aa, Felis catus
MHSSALLCFLVFLAGVGASRHQSTLSEDNCTHFSVSLPHMLRELRAAFGKVKTFFQTKDELHSILLTRSLLEDFKGYLGCQALSEMIQFYLEEVMPQAENEDPDIKQHVNSLGEKLKTLRLRLRRCHRFLPCENKSKVVEQVKSTFSKLQEKGVYKAMGEFDIFINYIEAYMTMKMKI

SEQ ID NO: 9 - protein, 178 aa, Cavia porcellus
MPGSALLCCLALLAGVKASQGTNTQSEDSCAHFPAGLPHMLRELRAAFGRVKTFFQTQDQLDNVLLNKSLLEDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 10 - protein, 178 aa, Lama glama
MPRSALLCCLILLAGVAASRDQGTQSENSCAHFPASLPHMLRELRAAFGRVKTFFQMKDQLDNMLLTRSLLEDFKGYLGCQALSEMIQFYLEEVMPQAENHGPDIKEHVNSLGEKLKTLRLRLRRCHRFLPCENKSKAVEQVRGVFSKLQEKGVYKAMSEFDIFINYIEAYMTMKMKN

SEQ ID NO: 11 - protein, 178 aa, Meriones unguiculatus
MPSSALLYCLILLAGVRPSRGEYPRNESNCTHFPVSQTHMLRELRAAFSQVKTFFQKKDQLDNILLTDSLQQDFKGYLGCQALSEMIQFYLVEVMPQAENHGPEIKENLNSLGEKLKTLRMQLRRCHRFLPCENKSKAVEQVKNDFNKLQEKGVYKAMNEFDIFINCIEAYMTIKMKS

SEQ ID NO: 12 - protein, 178 aa, Pan troglodytes
  VARIANT 84: note = X can be any naturally occurring amino acid
MHSSALLCCLVLLTGVRASPGQGTQSENSCTHFPGNLPNMLRDLRDAFSRVKTFFQMKDQLDNLLLKESLLEDFKGYLGCQALXEMIQFYLEEVMPQAENQDPDIKVHVNSLGENLKTLRLRLRRCHRFLPCENKSKAVEQVKNAFNKLQEKGIYKAMSEFDIFINYIEAYMTMKIRN

SEQ ID NO: 13 - protein, 178 aa, Macaca mulatta
MHSSALLCCLVLLTGVRASPGQGTQSENSCTRFPGNLPHMLRDLRDAFSRVKTFFQMKDQLDNILLKESLLEDFKGYLGCQALSEMIQFYLEEVMPQAENHDPDIKEHVNSLGENLKTLRLRLRRCHRFLPCENKSKAVEQVKNAFSKLQEKGVYKAMSEFDIFINYIEAYMTMKIQN

SEQ ID NO: 14 - protein, 178 aa, Callithrix jacchus
MHSSALLCCLVFLTGVRASPGQGTQSENSCTHFPGSLPHMLRELRVAFSRVKTFFQKKDQLDSMLLKESLLEDFKGYLGCQALSEMIQFYLEEVMPQAENHDPDIKEHVNSLGEKLKTFRLRLRRCHRFLPCENKSKAVVQVKNAVSKLQEKGIYKAMSEFDIFIDYIEAYMTMKAQN

SEQ ID NO: 15 - protein, 178 aa, Macaca nemestrina
MHSSALLCCLVLLTGVRASPGQGTQSENSCTRFPGNLPHMLRDLRDAFSRVKTFFQMKDQLDNILLKESLLEDFKGYLGCQALSEMIQFYLEEVMPQAENHDPDIKEHVNSLGENLKTLRLRLRRCHRFLPCENKSKAVEQVMNAFSKLQEKGVYKAMSEFDIFINYIEAYMTMKIQN

SEQ ID NO: 16 - protein, 178 aa, Macaca fascicularis
MHSSALLCCLVLLTGVRASPGQGTQSENSCTRFPGNLPHMLRDLRDAFSRVKTFFQMKDQLDNILLKESLLEDFKGYLGCQALSEMIQFYLEEVMPQAENHDPDIKEHVNSLGENLKTLRLRLRRCHRFLPCENKSKAVEQVKNAFSKLQEKGVYKAMSEFDIFINYIEAYMTMKIRN

SEQ ID NO: 17 - protein, 178 aa, Cercocebus atys
MHSSALLCCLVLLTGVRASPGQGTQSENSCTRFPGNLPHMLRDLRDVFSRVKTFFQMKDQLDNILLKESLLEDFKGYLGCQALSEMIQFYLEEVMPQAENHDPDIKEHVNSLGENLKTLRLRLRRCHRFLPCENKSKAVEQVKNAFSKLQEKGVYKAMSEFDIFINYIEAYMTMKIQN

SEQ ID NO: 18 - protein, 178 aa, Saimiri sciureus
MHSSALLCCLVFLTGVRASPGQGTQSENSCTHFPGSLPHMLRELRVAFGRVKTFFQKKDQLDSMLLKESLLEDFKGYLGCQALSEMIQFYLEEVMPQAENHDPDIKEHVNSLGEKLKTFRLRLRRCHRFLPCENKSKAVAQVKNAVSKLQEKGVYKAMSEFDIFIDYIEAYMTMKTQN

SEQ ID NO: 19 - protein, 177 aa, Capra hircus
MPSSSALLCCLVFLAGVAASRDASTLSDSSCTHFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 20 - protein, 177 aa, Ovis aries
MPSSSAVLCCLVFLAGVAASRDASTLSDSSCTHFPASLPHMLRDVRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 21 - protein, 181 aa, unidentified
  source 1..181: note = Canis familiaris
MHGSALLCCCLVLLAGVGASRHQSTLLEDDCTHFPASLPHMLRELRAAFGRVKIFFQMKDKLDNILLTGSLLEDFKSYLGCQALSEMIQFYLEEVMPRAENHDPDIKNHVNSLGEKLKTLRLRLRLRRCHRFLPCENKSKAVEQVKSAFSKLQEKGVYKAMSEFDIFINYIETYMTMRMKI

SEQ ID NO: 22 - protein, 220 aa, unidentified
  source 1..220: note = Mustela furo
MNGEASPHPHIKGGPVGEGLHTRGLLLQHQTTRPALGRQSFTMPSPALLCCLVLLAGVGASRHQSALSEDNCTHFPASLPHMLRELRAAFGRVKTFFQMKDKLGSILLTGSLLEDFKGYLGCQALSEMIQFYLEEVMPQAENHDPEVKEHVNSLGEKLKTLRLRLRRCHRFLPCENKSKAVEQVKSAFSKLQERGVYKAMSEFDIFINYIETYMTMRMKI

SEQ ID NO: 23 - protein, 178 aa, Oryctolagus cuniculus
MLSSALLCCLVFLGGTGASRGQDTPAENSCIHFPGGLPHMLRELRAAFGRVKTFFQSKDQLNSMLLTESLLEDLKGYLGCQALSEMIQFYLKDVMPQAENHSPAIREHVNSLGENLKTLRLRLRQCHRFLPCENKSKAVEQVKSAFSKLQEEGVYKAMSEFDIFINYIETYMTMKIKS

SEQ ID NO: 24 - protein, 178 aa, Mesocricetus auratus
MLGSALLCCLLLLAGVGPSRGQYTQHESNCTHFPVSQTHMLRELRTAFSQVKTFFQKKDQLDNILLTDSLLQDFKGYLGCQTLSEMIQFYLVEVMPQAENHGPEIKEHLNSLGEKLKTLRRQLQRCHRFLPCENKSKAVEQVKDNFNKLQEKGVFKAMNEFDIFINCIEVYMTIKMKS

SEQ ID NO: 25 - protein, 178 aa, unidentified
  source 1..178: note = Cricetulus griseus
MPVSALLCCLLLLAGVGPSRGQYTQQENNCTHFPVSQTHMLRELRTAFSQVKTFFQKKDQLDNILLTDSLVQDFEGYLGCQTLSEMIQFYLVEVMPQAENHGPEIKEHLNSLGEKLKTLRRQLQRCHRFLPCENKSKAVEKVKSDFNKLQEKGVYKAMNEFDIFINCIETYMTIKMKS

SEQ ID NO: 26 - protein, 15 aa, synthetic construct
  REGION 1..15: note = Synthetic
LRRCHRFLPCENKSK

SEQ ID NO: 27 - DNA, 538 bp, synthetic construct
  misc_feature 1..538: note = Synthetic
  source 1..538: mol_type = other DNA
atgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatctcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgag

SEQ ID NO: 28 - DNA, 538 bp, Homo sapiens
  source 1..538: mol_type = genomic DNA
atgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgag

SEQ ID NO: 29 - DNA, 7096 bp, synthetic construct
  misc_feature 1..7096: note = Synthetic
  source 1..7096: mol_type = other DNA
agctttgcagccgggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtaattgaaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtaaccgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagggctgcagcatgcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctggtggggtagggatgagggagggaggggcattgtgatgtacagggctgctctgtgagatcaagggtctcttaagggtgggagctggggcagggactacgagagcagccagatgggctgaaagtggaactcaaggggtttctggcacctacctacctgcttcccgctggggggtggggagttggcccagagtcttaagattggggcagggtggagaggtgggctcttcctgcttcccactcatcttatagctttctttccccagatccgaattcgagatccaaaccaaggaggaaaggatatcacagaggagacctcgagcccatcaacaagtttgtacaaaaaagcaggcaccatgctgccgtgcctcgtagtgctgctggcggcgctcctcagcctccgtcttggctcagacgctcatgggacagagctgcccagccctccgtctgtgtggtttgaagcagaatttttccaccacatcctccactggacacccatcccaaatcagtctgaaagtacctgctatgaagtggcgctcctgaggtatggaatagagtcctggaactccatctccaactgtagccagaccctgtcctatgaccttaccgcagtgaccttggacctgtaccacagcaatggctaccgggccagagtgcgggctgtggacggcagccggcactccaactggaccgtcaccaacacccgcttctctgtggatgaagtgactctgacagttggcagtgtgaacctagagatccacaatggcttcatcctcgggaagattcagctacccaggcccaagatggcccccgcaaatgacacatatgaaagcatcttcagtcacttccgagagtatgagattgccattcgcaaggtgccgggaaacttcacgttcacacacaagaaagtaaaacatgaaaacttcagcctcctaacctctggagaagtgggagagttctgtgtccaggtgaaaccatctgtcgcttcccgaagtaacaaggggatgtggtctaaagaggagtgcatctccctcaccaggcagtatttcaccgtgaccaacgtcatcatcttctttgcctttgtcctgctgctctccggagccctcgcctactgcctggccctccagctgtatgtgcggcgccgaaagaagctacccagtgtcctgctcttcaagaagcccagccccttcatcttcatcagccagcgtccctccccagagacccaagacaccatccacccgcttgatgaggaggcctttttgaaggtgtccccagagctgaagaacttggacctgcacggcagcacagacagtggctttggcagcaccaagccatccctgcagactgaagagccccagttcctcctccctgaccctcacccccaggctgacagaacgctgggaaacggggagccccctgtgctgggggacagctgcagtagtggcagcagcaatagcacagacagcgggatctgcctgcaggagcccagcctgagccccagcacagggcccacctgggagcaacaggtggggagcaacagcaggggccaggatgacagtggcattgacttagttcaaaactctgagggccgggctggggacacacagggtggctcggccttgggccaccacagtcccccggagcctgaggtgcctggggaagaagacccagctgctgtggcattccagggttacctgaggcagaccagatgtgctgaagagaaggcaaccaagacaggctgcctggaggaagaatcgcccttgacagatggccttggccccaaattcgggagatgcctggttgatgaggcaggcttgcatccaccagccctggccaagggctatttgaaacaggatcctctagaaatgactctggcttcctcaggggccccaacgggacagtggaaccagcccactgaggaatggtcactcctggccttgagcagctgcagtgacctgggaatatctgactggagctttgcccatgaccttgcccctctaggctgtgtggcagccccaggtggtctcctgggcagctttaactcagacctggtcaccctgcccctcatctctagcctgcagtcaagtgagcgggccaagcggcagctgctgaacttcgacctgctgaagctggccggcgacgtggagagcaaccccgggcccatgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatctcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgaacccagctttcttgtacaaagtggtgctagcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtaccggttagtaatgatcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggcgatggtacccaattcgccctatagtgagtcgtattacgcgcgcagcggccgaccatggcccaacttgtttattgcagcttataatggttacaaataaagcaatagcatcacaaatttcacaaataaagcatttttttcactgcattctagttgtggtttgtccaaactcatcaatgtatcttatcatgtctggatctccggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggtctgagacaataaccctgataaatgcttcaataatgta